(19) United States
(12) Patent Application Publication
(10) Pub. No.: US 2026/0218138 A1
(43) Pub. Date: Jul. 30, 2026

(54) ENGINEERED ACYLTRANSFERASES HAVING ALTERED SUBSTRATE SPECIFICITY

(71) Applicant: KEZBIOSOLUTIONS GMBH, Potsdam (DE)

(72) Inventors: Alexander Rittner, Potsdam-Golm (GE); Jan Degen, Potsdam-Golm (DE); Mirk Joppe, Potsdam-Golm (DE)

(73) Assignee: KEZ.BIOSOLUTIONS GMBH, Potsdam (DE)

(21) Appl. No.: 19/143,126

(22) PCT Filed: Jan. 2, 2024
(86) PCT No.: PCT/EP2024/050034

§ 371 Date: Jun. 25, 2025

(30) Foreign Application Priority Data
Dec. 30, 2022 (EP) 22217314.8

(51) Int. Cl. C12N 9/10; C12P 7/26; C12P 7/6409; C12P 21/00

(52) U.S. Cl. CPC C12N 9/1029; C12P 7/26; C12P 7/6409; C12P 21/00

(57) ABSTRACT

The present invention relates to the fields of fatty acid- and polyketide synthesis and protein engineering. In particular, it relates to the provision of engineered acyltransferases having altered substrate specificity as well as means and methods of using acyltransferases of the present invention for polyketide synthesis.

### TECHNICAL FIELD

The present invention relates to the fields of fatty acid- and polyketide synthesis, and protein engineering. In particular, it relates to the provision of engineered acyltransferases having altered substrate specificity as well as means and methods of using acyltransferases of the present invention for polyketide synthesis.

### BACKGROUND

Polyketides represent a versatile and important class of biomolecules. Particularly polyketide antibiotics, immunosuppressants, antiparasitics, as well as antifungal, cholesterol-lowering, and antitumoral agents represent important classes of biomolecules of outstanding therapeutic interest.

Despite having a biomolecular target, most polyketides are currently not used as therapeutics so that only a tiny fraction of their great potential is used for this particular substance class. In order to make them applicable for the human body, polyketides need to be tailored with certain functionalities. However, organic synthesis is very challenging and economically unfeasible for this particular substance class, due to their large carbon scaffolds with many functional groups and stereochemical properties. There is thus a great need in the pharmaceutical and biotechnological industry to provide systems allowing the synthesis of new and particularly custom-made polyketides with tailored functionalities in a reasonable time.

While polyketide synthase (PKS) systems bear a vast potential for synthetizing myriads of different biologically active compounds for drug design and other application, PKS and fatty acid synthase (FAS) enzymes are limited to substrates bound to Coenzyme A (CoA). CoA is composed of cysteamine (β-mercaptoethylamine), β-alanine, pantoic acid, organophosphate anhydride and a 3′-phosphoadenosine. The thiol group of the cysteamine can form a “high energy” thioester bond with carboxylic acids thereby forming “activated” acyl-CoA molecules.

CoA, however, is an abundant molecule in typical production strains. Consequently, having enzymes available with a decreased or even abolished specificity for CoA could be advantageous for the in vivo production of polyketides during metabolic engineering to shift the product yield towards a desired ketide or polyketide compound of interest using specifically designed enzymes as engineered molecular machines. In addition, using an artificial pathway (relying on acyl substrates bound to a different coenzyme moiety) for the biosynthesis of a target compound, avoids interference with the natural metabolism and a new functionality does no longer require identifying and establishing a suitable natural metabolic pathway to produce the substrate.

To our knowledge, there is no known acyltransferase that cannot bind CoA or would bind other coenzyme moieties. Re-engineering the coenzyme-binding properties, as in the present invention, is a highly challenging task and requires in-depth analysis, understanding and integration of the structural and functional properties as well as sequence-based information relating to the enzyme in context of the substrate binding.

Especially, as the nucleotide binding pocket only forms during or after substrate binding and follows the “induced fit” concept. Hence, the binding pocket is only visible in crystal structures of acyltransferases in complex with their respective CoA ester substrates. This includes the identification of charged residues participating in electrostatic binding of the phosphate groups.

Crystal structures of some FAS enzymes and PKS systems have been solved (Smith and Tsai 2007, Rittner et al. 2020). However, it has (to our knowledge) never been envisioned that the CoA binding pocket of an acyltransferase may be artificially redesigned so that it, ideally, does not use acyl-CoA substrates and accepts subunits bound to different coenzyme moieties, such as synthetic coenzyme moieties.

It was thus an object of the present invention to provide re-engineered acyltransferases, in particular those that are polyspecific in respect to the acyl moiety, for assisting in the production of polyketides and polyketide-precursors via biotechnological means to expand the currently available production methods. It was a particular object to expand and re-define the substrate recognition, binding and catalysis spectrum of acyltransferases to provide new acyltransferase enzymes, termed Selectases, capable of accepting a wide range of substrates, ideally while excluding CoA, to allow for targeted polyketide(-precursor) synthesis, preferably the synthesis of ketides or polyketide building blocks comprising positions within the polyketide molecule, where synthetic moieties can be inserted or added already during synthesis in vivo when produced by a host cell of interest or in vitro when produced in an artificial enzymatic system, or by semi-synthetic processes after production of the (poly-)ketide scaffold.

### Definitions

An “acyltransferase” as used herein refers to an acyltransferase, including malonyl-acetyl-transferases (MATs), within and/or originating from a fatty acid synthase (FAS) or a polyketide synthase (PKS), wherein an acyltransferase of the present invention, also referred to as “Selectase” herein, is a reprogrammed acyltransferase originating from a FAS. An acyltransferase can selectively bind and trans-esterify different coenzyme-bound acyl molecules onto an Acyl carrier protein (ACP), for instance as part of fatty acid or polyketide synthesis. While natural acyltransferases, including FAS malonyl-acetyl-transferases (MATs) are specific for Coenzyme A (CoA)-bound acyl molecules, an acyltransferase of the present invention is an artificially reprogrammed acyltransferase that no longer accepts Acyl-CoA substrates and can instead bind and trans-esterify acyl groups bound to other coenzyme moieties, including synthetic coenzyme moieties. An acyltransferase of the present invention may be expressed as part of a FAS, or a sub-part thereof, as a separate molecule and/or as part of a fusion protein.

An acyltransferase “originating” from a fatty acid synthase refers to an acyltransferase for which the corresponding, wild type MAT is part of said fatty acid synthase in nature, wherein an acyltransferase of the present invention may be present as part of said fatty acid synthase or may be an isolated acyltransferase according to the present disclosure. For instance, for the reprogrammed acyltransferase according to SEQ ID NO: 57 the corresponding wild type MAT sequence is SEQ ID NO: 29, which is part of the fatty acid synthase according to SEQ ID NO: 1; hence the reprogrammed acyltransferase according to SEQ ID NO: 57 originates from the fatty acid sequence according to SEQ ID NO: 1. Various wild type MATs and the fatty acid synthases they are part of in nature, are disclosed as SEQ ID NO: 29 to 56 and SEQ ID NO: 1 to 28, respectively. The skilled person can easily determine, for instance through sequence alignments, the acyltransferase within a given FAS sequence. An acyltransferase may also be any fragment, part and/or (sub)domain of a sequence according to SEQ ID NO: 29 to 56 or a corresponding sequence of a different FAS, as long as it has acyltransferase activity.

An “isolated acyltransferase” as used herein, refers to an acyltransferase that is not present as part of a continuous polypeptide comprising the complete fatty acid synthase from which it originates, optionally not comprising more than the acyltransferase and optionally the linker domain (LD), or a part thereof, and/or the ketoacyl synthase (KS) of the fatty acid synthase from which it originates. An isolated acyltransferase may be present as a discrete enzyme, i.e. a polypeptide consisting of said acyltransferase. Preferably, a polypeptide comprising an isolated acyltransferase further comprises at least one protein, domain or part thereof to increase the stability, such as an LD, or a part thereof, wherein the LD may originate from the same FAS or may originate, in form of a fusion protein, from at least one different FAS and/or a polyketide synthase (PKS), or a different protein, domain or part thereof, for example an MBP-tag or a different tag. In certain embodiments, an isolated acyltransferase is present as part of a polypeptide comprising the acyltransferase, a KS as well as a LD connecting the acyltransferase and the KS, wherein the KS, the LD may originate from the same FAS or may originate, in form of a fusion protein, from at least one different FAS and/or a PKS. Generally, an isolated acyltransferase may be present as part of a fusion protein further comprising at least one polypeptide that does not originate from the same FAS or does not originate from any FAS, such one or more different enzyme parts and/or domains, including PKS functional domains, one or more tags, one or more binding domains, synthetic zippers and the like.

An “amino acid exchange” as used herein, refers to the presence of an amino acid at a given position of the primary amino acid sequence of a polypeptide that differs from the amino acid present at said position of the primary amino acid sequence of a reference polypeptide, such as a wild type polypeptide. Commonly, amino acid exchanges are achieved by mutating the respective codon of a nucleic acid sequence encoding the polypeptide into a codon encoding a different amino acid at said position. Means and methods to exchange an amino acid at a desired position, for instance but not limited to PCR-based cloning methods as disclosed herein, are well established in the field and available to the skilled person.

A “fatty acid synthase” or “FAS” as used herein, refers to an animal, including human, fatty acid synthase of type I, being a multi-functional protein, also called a multi-enzyme, capable of catalyzing all steps of fatty acid synthesis. In nature, wild type fatty acid synthases catalyze multiple rounds of Claisen condensation reactions with acetyl-CoA and malonyl-CoA recognized and transferred by an acyltransferase being a malonyl-acetyl-transferase (MAT) as substrates to synthesize fatty acids. The large multi-functional proteins comprise the functional domains of an acyltransferase transferring the acyl-substrates onto an acyl carrier protein (ACP), a ketoacyl synthase (KS) catalyzing the Claisen condensation reaction, a ketoacyl reductase (KR), hydroxyacyl dehydratase (DH) and enoyl reductase (ER) creating a fully saturated acyl backbone and a thioesterase (TE) releasing the synthesized fatty acid. (Smith and Tsai 2007).

A “fusion protein” as used herein refers to a polypeptide that is created by joining at least two different polypeptides, which may each be a complete protein existing in nature, a part thereof, or a fully synthetic polypeptide, to form one continuous artificial polypeptide. Commonly, a fusion protein is produced by fusing the nucleic acid sequences of the polypeptides to be joined. Means and methods to clone and express fusion proteins are well-established in the field and available to the skilled person. Fusion proteins of the present disclosure may for instance comprise one or more tags, such as affinity tags, including at least one His-tag, strep-tag, SNAP-tag, HA-tag and the like, or combinations thereof, e.g., an N-terminal His-tag and a C-terminally located strep-tag, or vice versa, epitope tags, fluorescence tags, solubilization tags, synthetic zippers or the like. A cleavable tag that can be removed after production may be used as well. Usually, tags will be located at the N- or C-terminus of a polypeptide not to disturb the function of the folded protein, but in certain cases, a tag may also be located within a polypeptide sequence (e.g., forming a discrete structural epitope not disturbing the fold and function of the polypeptide the tag is inserted in). Further, one or more linker sequences may be used, including a FAS and/or PKS LD, and/or other linkers (including synthetic linkers) or spacers or flexible linkers to connect the different entities of a fusion molecule with each other so that the different entities can exert there function appropriately (e.g., “GA” linkers or spacers of various length building alpha-helical structures may be used, one or more binding or docking domains, one or more dimerization domains, such as leucine zippers).

Whenever the present disclosure relates to the percentage of the identity of nucleic acid or amino acid sequences, this identity is determined by a comparison of a sequence of interest, the reference sequence (e.g., a SEQ ID NO as disclosed herein), to another sequence, the query sequence, over the entire length of the reference sequence. Identity is obtained by using the EMBOSS Water Pairwise Sequence Alignments (nucleotide) program or the EMBOSS Water Pairwise Sequence Alignments (protein) program (www.ebi.ac.uk/Tools/psa/emboss_water/) for amino acid sequences. Those tools provided by the European Molecular Biology Laboratory (EMBL) European Bioinformatics Institute (EBI) for local sequence alignments use a modified Smith-Waterman algorithm (see www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

A “linker domain” or “LD” as used herein refers to both linker sequences upstream and downstream of an acyltransferase within a FAS or PKS that—together—link the acyltransferase to the KS, both sub-parts also being referred to as KS-AT (or KS-MAT) Linker and post-AT Linker. A linker domain as disclosed herein may comprise or consist of both sub-parts from the same FAS or PKS or a linker domain may be hybrid comprising or consisting of a KS-(M)AT Linker and a post-AT Linker, each stemming from a different FAS or PKS.

A “negatively charged” amino acid as used herein, refers to aspartate, glutamate, histidine or a non-natural amino acid or amino acid analog carrying a negative net charge at least at certain pH conditions. The terms “aspartate” and “glutamate” are used interchangeably with the terms “aspartic acid” and “glutamic acid”, respectively, herein, irrespective of the protonation state.

Notably, histidine can be negatively or positively charged depending on the pH. Further, a neutral charge already at pH~8.0 is possible. Therefore, depending on the pH and in view of the pl, histidine can be qualified as negatively charged residue, but also differently.

A “reference position” as used herein, refers to one of the conserved positions (i), (ii), (iii), (iv), (v), (vi), (vii), (viii) or (ix) as defined in Table 1. For an acyltransferase originating, for example, from a murine FAS according to SEQ ID NO: 1, the reference positions (i) to (ix) correspond to the positions D160, T161, F184, R286, K186, K285, R300, A282, and G137 with the numbering based on the sequence of the acyltransferase domain itself (SEQ ID NO: 29), which corresponds to positions D647, T648, F671, R773, K673, K772, R787, A769, and G624 with the numbering based on the sequence of the complete murine FAS (SEQ ID NO: 1). For an acyltransferase originating from a chicken FAS according to SEQ ID NO: 14, as a further example, the reference positions (i) to (viii) correspond to the positions D160, T161, F184, R286, K186, R285, K300, A282, and G137 with the numbering based on the sequence of the acyltransferase domain itself (SEQ ID NO: 42), which corresponds to positions D646, T647, F670, R772, K672, R771, K786, A768 and G623 with the numbering based on the sequence of the complete chicken FAS (SEQ ID NO: 14). When comparing different sequences of acyltransferases according to the present disclosure (e.g. SEQ ID NOs: 29 to 56) numbers of positions corresponding to the same reference position are frequently identical (when the numbering is based on the acyltransferase domain itself) but may also differ.

The reference positions disclosed herein are not limited to the sequences of SEQ ID NOs: 1 to 28 or SEQ ID NOs: 29 to 56. Sequence alignments, such as shown in Table 1, are well-established in the field and the skilled person can easily determine for any given FAS sequence, or the acyltransferase sequence therein, the positions of reference positions (i), (ii), (iii), (iv), (v), (vi), (vii), (viii) and/or (ix) within that sequence and further determine whether there is a threonine, serine or asparagine residue at reference position (i), a phenylalanine residue at reference position (ii), an arginine residue at reference position (iii), a lysine or arginine residue at reference position (iv), a lysine or arginine residue at reference position (v), a lysine or arginine residue at reference position (vi), an alanine residue at reference position (vii), a glycine residue at reference position (viii), and/or a aspartate or glutamate residue at reference position (ix).

Whenever the present disclosure refers to an “analogous” residue in the context to SEQ ID NO: 29, it refers to the residue present in a different sequence at the position that corresponds to the same reference position. For example, T161 of SEQ ID NO: 14 is an analogous threonine residue to T161 of SEQ ID NO: 29 and R285 of SEQ ID NO 14 is an analogous arginine residue to K285 of SEQ ID NO: 29. While the numbering based on SEQ ID NO: 14 is identical to the numbering based on SEQ ID NO: 29, an analogous residue may not necessarily have the identical number as the respective residue in SEQ ID NO: 29, depending on the individual sequence.

The terms “polypeptide” and “protein” are used interchangeably herein. Naturally, protein functionality, including enzymatic activity, is dependent on further factors, such as temperature, pH, salts, and the like and may require further proteins and/or co-factors as it is known to the skilled person. Polypeptides disclosed herein may start with an additional methionine residue resulting from translation of the start codon, or alternatively with a different additional residue, such as valine or leucine resulting from translation of an alternative start codon, likewise polypeptides disclosed herein as beginning with a methionine residue may also be present without said methionine e.g. if used in context of an N-terminal fusion to a tag or other polypeptide.

### BRIEF DESCRIPTION OF THE DRAWINGS

FIG. 1 (FIG. 1) shows a cartoon of the structure of a mammalian FAS homodimer with one of the Malonyl-Acetyl-Transferases (MAT) highlighted as well as a corresponding cartoon showing the position of the CoA binding channel within a MAT as a cartoon. The numbering of the residues of the active site (S94, H196 and N251) is based on SEQ ID NO: 29.

FIG. 2 (FIG. 2) shows the residues of a murine MAT (SEQ ID NO: 29) identified in the structural as well as sequence-based analysis for targeted reprogramming of the coenzyme A binding. Structural sketches of the eight different identified residues are shown in black with the positively charged residues (K186, K285 and R300) being marked with a black “+”. A structural sketch of the CoA is shown in gray, with the negatively charged phosphate groups being marked with a gray “−”. Pi-stacking between the CoA-Adenine and the residues F184 and R286 are indicated as “π”. H-bonding between residue T161 and the CoA-Adenine is indicated with a dotted line and an “H”.

FIG. 3 (FIG. 3) shows an exemplary scheme of engineering a Malonyl-Acetyl-Transferase (MAT) into a reprogrammed acyltransferase having altered substrate specificity. In the top left is a sketch of a KS-LD-MAT polypeptide capable of binding CoA. In the top right is a sketch of an engineered KS-LD-acyltransferase according to the present invention. On the bottom right are examples of using a reprogrammed acyltransferase of the present invention.

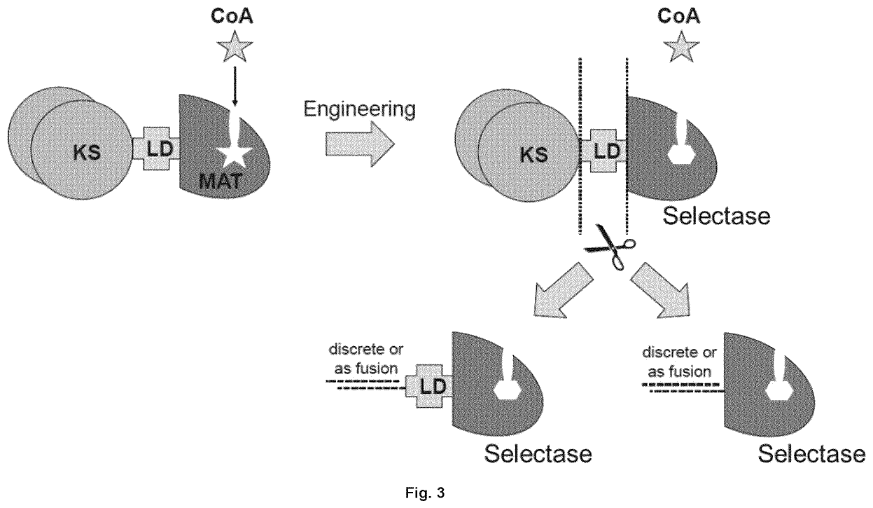

FIG. 4 (FIG. 4) shows turnover rates of fifty exemplary mutants on a logarithmic scale. Specific transfers from Mal-CoA to N-(2-mercaptoethyl)-hexanamide were analyzed at fixed substrate and acceptor concentrations. Y-axis shows turnover in s⁻¹ on a logarithmic scale. Mutant A38 was measured in two concentration groups and mutant A48 did not give a detectable signal under the used conditions. Shown error bares reflect standard deviations from triplicates.

FIG. 5 (FIG. 5) shows turnover rates for the transfer of malonyl moieties from a non-CoA substrate. Specific transfers from the non-CoA substrate 3-Oxo-3-[[2-[(1-oxohexyl)amino]ethyl]thio]-propanoic acid to N-(2-hydroxyethyl)-hexanamide were analyzed at fixed substrate and acceptor concentrations. Y-axis shows enzyme-specific turnover in s⁻¹. Measurements were performed in duplets.

FIG. 6 (FIG. 6) shows exemplary enzyme kinetics of the mutant A35 for the transfer of malonyl moieties to ACP from Mal-CoA (FIG. 6A) and 3-Oxo-3-[[2-[(1-oxohexyl)amino]ethyl]thio]-propanoic acid (FIG. 6B). Y-axes show turnover in s⁻¹ and x-axes show substrate concentration in μM. Apparent kinetic parameters at a fixed acceptor concentration of ACP of 85 μM were determined by fitting the data points with the Michaelis-Menten function. Shown error bares reflect standard deviations from triplicates.

### SUMMARY OF THE INVENTIONS

In a first aspect, there is provided an acyltransferase having altered substrate specificity, wherein the acyltransferase comprises one or more amino acid exchange(s) at reference position(s) (i) T161 according to SEQ ID NO: 29, or an analogous threonine, serine or asparagine residue at said reference position as defined in Table 1, wherein the amino acid is exchanged to alanine, glycine, valine, leucine, isoleucine, tyrosine, phenylalanine, tryptophan, aspartate or glutamate; and/or (ii) F184 according to SEQ ID NO: 29, or an analogous phenylalanine residue at said reference position as defined in Table 1, wherein the amino acid is exchanged to alanine, glycine, valine, leucine, isoleucine, methionine, aspartate or glutamate; and/or (iii) R286 according to SEQ ID NO: 29, or an analogous arginine residue at said reference position as defined in Table 1, wherein the amino acid is exchanged to alanine, glycine, valine, leucine, isoleucine, methionine, aspartate or glutamate; and/or (iv) K186 according to SEQ ID NO: 29, or an analogous lysine or arginine residue at said reference position as defined in Table 1, wherein the amino acid is exchanged to a negatively charged amino acid or to glycine, alanine, valine, leucine, isoleucine or methionine; and/or (v) K285 according to SEQ ID NO: 29, or an analogous lysine or arginine residue at said reference position as defined in Table 1, wherein the amino acid is exchanged to a negatively charged amino acid; and/or (vi) R300 according to SEQ ID NO: 29, or an analogous lysine or arginine residue at said reference position as defined in Table 1, wherein the amino acid is exchanged to a negatively charged amino acid, and/or (vii) A282 according to SEQ ID NO: 29, or an analogous alanine residue at said reference position as defined in Table 1, wherein the amino acid is exchanged to valine, leucine, isoleucine, phenylalanine or tyrosine; and/or (viii) G137 according to SEQ ID NO: 29, or an analogous glycine residue at said reference position as defined in Table 1, wherein the amino acid is exchanged to valine, leucine, isoleucine, phenylalanine or tyrosine.

In some embodiments of the first aspect, an amino acid exchange at reference position (i) is an exchange to leucine, alanine, tryptophan, or valine, preferably to leucine or alanine, more preferably to leucine; and/or wherein an amino acid exchange at reference position (ii) is an exchange to leucine, alanine, glycine, valine, or glutamate, preferably to leucine, alanine or glutamate, more preferably to leucine or alanine; and/or wherein an amino acid exchange at reference position (iii) is an exchange to leucine, alanine, glycine, or valine, preferably to leucine or alanine; and/or wherein an amino acid exchange at reference position (iv) is an exchange to a negatively charged amino acid, or to alanine or valine, preferably to glutamate or aspartate; and/or wherein an amino acid exchange at reference position (v) is an exchange to glutamate or aspartate; and/or wherein an amino acid exchange at reference position (vi) is an exchange to glutamate or aspartate; and/or wherein an amino acid exchange at reference position (vii) is an exchange to leucine, isoleucine, phenylalanine or tyrosine; and/or wherein an amino acid exchange at reference position (viii) is an exchange to leucine, isoleucine, phenylalanine or tyrosine, preferably to leucine.

In some embodiments of the first aspect, an amino acid exchange at reference position (i) is an exchange to leucine, isoleucine, tyrosine, phenylalanine or glycine; and/or wherein an amino acid exchange at reference position (ii) is an exchange to leucine, alanine, isoleucine or methionine; and/or wherein an amino acid exchange at reference position (iii) is an exchange leucine, isoleucine or methionine; and/or wherein an amino acid exchange at reference position (iv) is an exchange to alanine, glycine, valine, leucine, isoleucine or methionine; and/or wherein an amino acid exchange at reference position (vii) is an exchange to valine; and/or wherein an amino acid exchange at reference position (viii) is an exchange to valine.

In preferred embodiments of the first aspect, the acyltransferase is an isolated acyltransferase originating from a fatty acid synthase.

In some embodiments of the first aspect, the acyltransferase comprises amino acid exchanges as defined above at reference positions (iv) and (i), or (iv) and (ii), or (iv) and (iii), or (iv) and (v), or (iv) and (vi), or (iv) and (vii), or (iv) and (viii), or (iv) and two or more of reference positions of (i), (ii), (iii), (v), (vi), (vii) and/or (viii), optionally wherein the amino acid exchange at reference position (iv) is an amino acid exchange to glutamate or aspartate.

In some embodiments of the first aspect, the acyltransferase comprises amino acid exchanges as defined above at reference positions (i) and (ii), or (i) and (iii), or (i) and (iv), or (i) and (v), or (i) and (vi), or (i) and (vii), or (i) and (viii), or (i) and two or more of reference positions of (ii), (iii), (iv), (v), (vi), (vii) and/or (viii), optionally wherein the amino acid exchange at reference position (i) is an amino acid exchange to leucine, alanine, tryptophan, or valine, preferably to leucine or alanine, more preferably to leucine.

In some embodiments of the first aspect, the acyltransferase comprises amino acid exchanges as defined above at reference positions: (i), optionally wherein the amino acid exchange at reference position (i) is an amino acid exchange to leucine, alanine, tryptophan, or valine, preferably to leucine or alanine, more preferably to leucine.

In some embodiments, the acyltransferase further comprises amino acid exchanges as defined above at reference positions: (ii), optionally wherein the amino acid exchange at reference position (ii) is an amino acid exchange to leucine, alanine, glycine, valine, or glutamate, preferably to leucine, alanine or glutamate, more preferably to leucine or alanine; and (iii), optionally wherein the amino acid exchange at reference position (iii) is an amino acid exchange to leucine, alanine, glycine, or valine, preferably to leucine or alanine.

In some embodiments, the acyltransferase further comprises an amino acid exchange as defined above at reference (iv), optionally wherein the amino acid exchange at reference position (iv) is an amino acid exchange to glutamate or aspartate.

In some embodiments, the acyltransferase further comprises an amino acid exchange as defined above at reference (vi), optionally wherein the amino acid exchange at reference position (vi) is an amino acid exchange to glutamate or aspartate.

In some embodiments of the first aspect, the acyltransferase comprises amino acid exchanges to glutamate at reference positions (iv) and (vi), or amino acid exchanges to aspartate at reference positions (iv) and (vi), or an amino acid exchange to glutamate at reference position (iv) and an amino acid exchange to aspartate at reference position (vi), or an amino acid exchange to aspartate at reference position (iv) and an amino acid exchange to glutamate at reference position (vi).

In some embodiments of the first aspect, the acyltransferase comprises an amino acid exchange to glutamate or aspartate at reference position (iii) and optionally further comprises an amino acid exchange at reference position: (ix) D160 according to SEQ ID NO: 29, or an analogous aspartate or glutamate residue at said reference position as defined in Table 1, wherein the amino acid is exchanged to arginine, histidine, lysine, glutamine, or asparagine. In some embodiments, the amino acid exchange at reference position (ix) is an exchange to arginine. In other embodiments, the amino acid exchange at reference position (ix) is an exchange to histidine, lysine, glutamine, or asparagine.

In some embodiments of the first aspect, the acyltransferase originates from a fatty acid synthase of an organism selected from vertebrata, including aves and mammalia, including artiodactyla, perissodactyla, carnivora, primates and rodentia, preferably wherein the acyltransferase originates from a fatty acid synthase comprising or consisting of a wild type amino acid sequence selected from any one of SEQ ID NO: 1 to 28, or a sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity thereto.

In some embodiments of the first aspect, the acyltransferase comprises or consists of an amino acid sequence of any of SEQ ID NO: 57 to 82 or 111 to 133, or a sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity thereto.

In a second aspect, there is provided a fusion protein comprising at least one acyltransferase of any one of the embodiments of the first aspect, preferably wherein the fusion protein further comprises at least one linker domain and optionally at least one ketoacyl synthase domain.

In a third aspect, there is provided a nucleic acid encoding the acyltransferase of the first aspect, or the fusion protein of the third aspect.

In a fourth aspect, there is provided an expression construct or vector comprising at least one nucleic acid of the third aspect.

In a fifth aspect, there is provided a cell comprising at least one acyltransferase of the first aspect; and/or at least one fusion protein of the second aspect; and/or at least one nucleic acid of the third aspect; and/or at least one expression construct or vector of the fourth aspect.

In a sixth aspect, there is provided a kit comprising at least one acyltransferase of the first aspect; and/or at least one fusion protein of the second aspect; and/or at least one nucleic acid of the third aspect; and/or at least one expression construct or vector of the fourth aspect; and/or at least one cell of the fifth aspect; optionally further comprising at least one container; and/or a set of reagents including buffer, medium and/or substrate(s); and/or means of transfection. A kit will usually comprise all agents, buffers, co-factors etc. necessary to guarantee the functionality of the at least one acyltransferase in an assay of interest.

In a seventh aspect, there is provided a use of at least one acyltransferase as defined in the first aspect; and/or at least one fusion protein as defined in the second aspect; and/or at least one nucleic acid as defined in the third aspect; and/or at least one expression construct or vector as defined in the fourth aspect; and/or at least one cell as defined in the fifth aspect; and/or at least one kit as defined in the sixth aspect; for polyketide synthesis, fatty acid synthesis and/or non-ribosomal peptide synthesis, optionally wherein it is a use for reacting at least two different substrates.

### DETAILED DESCRIPTION

The present invention will now be described in detail based on the detailed description, including non-limiting Examples, the attached drawings and the sequences as provided with the sequence listing.

In a first aspect, there is provided an acyltransferase having altered substrate specificity, wherein the acyltransferase comprises one or more amino acid exchange(s) at reference position(s) (i) T161 according to SEQ ID NO: 29, or an analogous threonine, serine or asparagine residue at said reference position as defined in Table 1, wherein the amino acid is exchanged to alanine, glycine, valine, leucine, isoleucine, tyrosine, phenylalanine, tryptophan, aspartate or glutamate; and/or (ii) F184 according to SEQ ID NO: 29, or an analogous phenylalanine residue at said reference position as defined in Table 1, wherein the amino acid is exchanged to alanine, glycine, valine, leucine, isoleucine, methionine, aspartate or glutamate; and/or (iii) R286 according to SEQ ID NO: 29, or an analogous arginine residue at said reference position as defined in Table 1, wherein the amino acid is exchanged to alanine, glycine, valine, leucine, isoleucine, methionine, aspartate or glutamate; and/or (iv) K186 according to SEQ ID NO: 29, or an analogous lysine or arginine residue at said reference position as defined in Table 1, wherein the amino acid is exchanged to a negatively charged amino acid or to glycine, alanine, valine, leucine, isoleucine, methionine, phenylalanine, tyrosine, tryptophan, cysteine, serine, threonine, histidine or proline; and/or (v) K285 according to SEQ ID NO: 29, or an analogous lysine or arginine residue at said reference position as defined in Table 1, wherein the amino acid is exchanged to a negatively charged amino acid; and/or (vi) R300 according to SEQ ID NO: 29, or an analogous lysine or arginine residue at said reference position as defined in Table 1, wherein the amino acid is exchanged to a negatively charged amino acid, and/or (vii) A282 according to SEQ ID NO: 29, or an analogous alanine residue at said reference position as defined in Table 1, wherein the amino acid is exchanged to valine, leucine, isoleucine, phenylalanine or tyrosine; and/or (viii) G137 according to SEQ ID NO: 29, or an analogous glycine residue at said reference position as defined in Table 1, wherein the amino acid is exchanged to valine, leucine, isoleucine, phenylalanine or tyrosine.

Wild type (i.e. without comprising the one or more amino acid exchanges of the present invention) acyltransferases from fatty acid synthases can transfer different acyl-groups from acyl-coenzyme A (CoA) substrates to an acyl carrier protein. While they are polyspecific for multiple different acyl groups, they are specific for CoA-bound substrates. The two different types of specificity, the CoA specificity and the acyl polyspecificity, are conferred by different residues of the enzyme. The residues at the reference positions are crucial for binding to the CoA moiety of the substrate and the amino acid exchanges of the present invention can be used to alter these binding properties and engineer acyltransferases that can bind to substrates covalently bound to moieties other than CoA, including synthetic coenzyme moieties. Acyltransferases having altered substrate specificity according to the present invention are acyltransferases having a reduced ability, preferably essentially no ability, to transfer CoA-bound substrates, while the acyl polyspecificity may be unaltered.

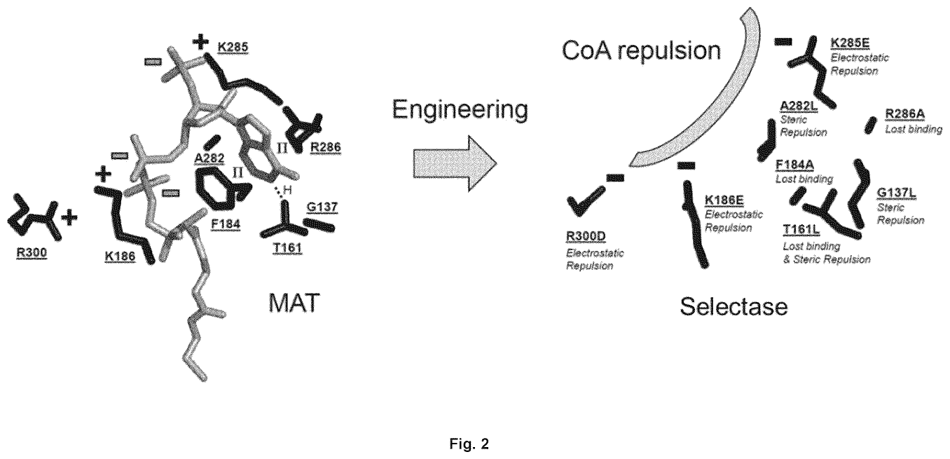

The residues at reference positions (i), (ii) and (iii) are crucial residues for the binding pocket of the respective coenzyme (see FIG. 2) and interact with the nucleobase portion of the CoA moiety through hydrogen bonding (reference position (i)) or pi-stacking (reference positions (ii) and (iii)). One or more amino acid exchange(s) at reference positions (i), (ii) and/or (iii) to alanine, glycine and/or valine may be used to prevent the H-bonding and/or Pi-stacking. An amino acid exchange at reference position (i) to leucine, isoleucine, tyrosine, phenylalanine, tryptophan, aspartate or glutamate may be used to sterically manipulate and reprogram the binding to coenzyme moieties, wherein an exchange to tyrosine, phenylalanine or tryptophan further may be used to stabilize the protein through pi-stacking. The amino acid exchange(s) at reference position (ii) and/or (iii) to leucine, isoleucine, methionine, aspartate or glutamate may be used to sterically manipulate the binding to coenzyme moieties.

The lysine or arginine residues at reference positions (iv), (v) and (vi) interact electrostatically with the negatively charged phosphate groups of CoA. Exchanging one or more residues at reference positions (iv), (v) and/or (vi) to one or more negatively charged residue(s) may be used to engineer acyltransferases that, instead, bind to one or more positively charged chemical groups of coenzyme moieties other than CoA, including synthetic coenzyme moieties.

An amino acid exchange at reference position (iv) to amino acid residues that are, at a neutral pH, not positively charged or uncharged, and optionally non-polar, such as alanine, glycine, valine, leucine, isoleucine, methionine, phenylalanine, tyrosine, tryptophan, cysteine, serine, threonine, histidine, proline, in particular alanine, glycine, valine, leucine, isoleucine or methionine may be used to artificially eliminate the electrostatic interaction at this reference position. Experimental results show that the effect of amino acid exchanges at reference position (iv) are highly addictive with amino acid exchanges at reference position, (i), (ii), (iii) and (vi) and suggest that the most important factor in respect to reference position (iv) is the elimination of the naturally occurring positive charge at this position, while the exact nature of the amino acid exchange or the introduction of a negative charge at this position are less relevant than the elimination of the positive charge.

The alanine residue at reference position (vii) and the glycine residue at reference position (viii) form structural parts of the coenzyme binding pocket. Exchanging the residues at one and/or both of these positions may be used to manipulate the structural shape of the coenzyme binding pocket through modification of the steric arrangement.

In the wild-type protein, the aspartate or glutamate residue at reference position (ix) forms an H-bond with the arginine residue at reference position (iii). In embodiments in which the arginine, comprising a hydrogen donor residue, at reference position (iii) is exchanged to an aspartate or glutamate, comprising a hydrogen acceptor residue, it is favorable also to exchange the aspartate or glutamate at reference position (ix) to an amino acid comprising a hydrogen donor residue, preferably to arginine, histidine, lysine, glutamine, or asparagine. This allows H-bond formation between mutated residues (iii) and (ix) with reversed hydrogen donor/acceptor roles and can serve to stabilize the engineered acyltransferase.

Preferred positions for engineering a Selectase of the present invention are reference positions (i), (ii), (iii), (iv), (v) and/or (vi), alone or in combination, even more preferred is a combination of any one of reference position(s) (ii) and (iii), alone or preferred in combination, or a combination of any one of reference position(s) (i) and (iv), alone or in combination, preferably in combination with at least one further engineering at another position.

In some embodiments of the first aspect, an amino acid exchange at reference position (i) is an exchange to leucine, alanine, tryptophan, or valine, preferably to leucine or alanine, more preferably to leucine; and/or wherein an amino acid exchange at reference position (ii) is an exchange to leucine, alanine, glycine, valine, or glutamate, preferably to leucine, alanine or glutamate, more preferably to leucine or alanine; and/or wherein an amino acid exchange at reference position (iii) is an exchange to leucine, alanine, glycine, or valine, preferably to leucine or alanine; and/or wherein an amino acid exchange at reference position (iv) is an exchange to a negatively charged amino acid, or to alanine or valine, preferably to glutamate or aspartate; and/or wherein an amino acid exchange at reference position (v) is an exchange to glutamate or aspartate; and/or wherein an amino acid exchange at reference position (vi) is an exchange to glutamate or aspartate; and/or wherein an amino acid exchange at reference position (vii) is an exchange to leucine, isoleucine, phenylalanine or tyrosine; and/or wherein an amino acid exchange at reference position (viii) is an exchange to leucine, isoleucine, phenylalanine or tyrosine, preferably to leucine.

In some embodiments of the first aspect, an amino acid exchange at reference position (i) is an exchange to leucine, isoleucine, tyrosine, phenylalanine or glycine; and/or wherein an amino acid exchange at reference position (ii) is an exchange to leucine, alanine, isoleucine or methionine; and/or wherein an amino acid exchange at reference position (iii) is an exchange to leucine, isoleucine or methionine; and/or wherein an amino acid exchange at reference position (iv) is an exchange to alanine, glycine, valine, leucine, isoleucine or methionine; and/or wherein an amino acid exchange at reference position (vii) is an exchange to valine; and/or wherein an amino acid exchange at reference position (viii) is an exchange to valine.

In preferred embodiments of the first aspect, the acyltransferase is an isolated acyltransferase originating from a fatty acid synthase.

In one embodiment, the isolated acyltransferase is a polypeptide or part of a polypeptide comprising or consisting of the acyltransferase, without comprising further parts and/or domains, including a LD, of the FAS from which it originates. The isolated acyltransferase may be part of a fusion protein according to the second aspect.

In another embodiment, the isolated acyltransferase is part of a polypeptide comprising or consisting of the acyltransferase and a LD, or a part thereof, without comprising further parts and/or domains of the FAS from which it originates. The isolated acyltransferase may be part of a fusion protein according to the second aspect.

In another embodiment, the isolated acyltransferase is part of a polypeptide comprising or consisting of the portion of a FAS spanning the ketoacyl synthase (KS), the acyltransferase and the LD connecting the KS and the acyltransferase, without comprising further parts and/or domains of the FAS from which it originates. Such a KS-LD-acyltransferase may be part of a fusion protein according to the second aspect.

In some embodiments of the first aspect, the acyltransferase comprises amino acid exchanges as defined above at reference positions (iv) and (i), or (iv) and (ii), or (iv) and (iii), or (iv) and (v), or (iv) and (vi), or (iv) and (vii), or (iv) and (viii), or (iv) and two or more of reference positions of (i), (ii), (iii), (v), (vi), (vii) and/or (viii), optionally wherein the amino acid exchange at reference position (iv) is an amino acid exchange to glutamate or aspartate.

In some embodiments of the first aspect, the acyltransferase comprises amino acid exchanges as defined above at reference positions (i) and (ii), or (i) and (iii), or (i) and (iv), or (i) and (v), or (i) and (vi), or (i) and (vii), or (i) and (viii), or (i) and two or more of reference positions of (ii), (iii), (iv), (v), (vi), (vii) and/or (viii), optionally wherein the amino acid exchange at reference position (i) is an amino acid exchange to leucine, alanine, tryptophan, or valine, preferably to leucine or alanine, more preferably to leucine.

In preferred embodiments, the acyltransferase comprises amino acid exchanges as defined above at reference positions (i), (ii), (iii), (iv), and optionally also at reference position (vi).

In some embodiments of the first aspect, the acyltransferase comprises amino acid exchanges as defined above at reference positions: (i), optionally wherein the amino acid exchange at reference position (i) is an amino acid exchange to leucine, alanine, tryptophan, or valine, preferably to leucine or alanine, more preferably to leucine.

In some embodiments, the acyltransferase further comprises amino acid exchanges as defined above at reference positions: (ii), optionally wherein the amino acid exchange at reference position (ii) is an amino acid exchange to leucine, alanine, glycine, valine, or glutamate, preferably to leucine, alanine or glutamate, more preferably to leucine or alanine; and (iii), optionally wherein the amino acid exchange at reference position (iii) is an amino acid exchange to leucine, alanine, glycine, or valine, preferably to leucine or alanine.

In some embodiments, the acyltransferase further comprises an amino acid exchange as defined above at reference (iv), optionally wherein the amino acid exchange at reference position (iv) is an amino acid exchange to glutamate or aspartate.

In some embodiments, the acyltransferase further comprises an amino acid exchange as defined above at reference (vi), optionally wherein the amino acid exchange at reference position (vi) is an amino acid exchange to glutamate or aspartate.

In some embodiments of the first aspect, the acyltransferase comprises amino acid exchanges to glutamate at reference positions (iv) and (vi), or amino acid exchanges to aspartate at reference positions (iv) and (vi), or an amino acid exchange to glutamate at reference position (iv) and an amino acid exchange to aspartate at reference position (vi), or an amino acid exchange to aspartate at reference position (iv) and an amino acid exchange to glutamate at reference position (vi).

In one embodiment of the first aspect, the acyltransferase comprises an amino acid exchange to aspartate at reference position (i) and an amino acid exchange to glutamate at reference position (ii).

In one embodiment of the first aspect, the acyltransferase comprises an amino acid exchange to leucine at least at reference positions (i), (ii) and (iii).

In one embodiment of the first aspect, the acyltransferase comprises an amino acid exchange to alanine at reference positions (i), (ii) and (iii), and an amino acid exchange to aspartate or glutamate at reference position (iv).

In one embodiment of the first aspect, the acyltransferase comprises an amino acid exchange to alanine at reference positions (i), (ii) and (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi).

In one embodiment of the first aspect, the acyltransferase comprises an amino acid exchange to leucine at reference positions (i), (ii) and (iii); and an amino acid exchange to glutamate or aspartate at reference position (iv).

In one embodiment of the first aspect, the acyltransferase comprises an amino acid exchange to leucine at reference positions (i), (ii) and (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi).

In one embodiment of the first aspect, the acyltransferase comprises an amino acid exchange to leucine at reference positions (i) and an amino acid exchange to alanine at reference positions (ii) and (iii); and an amino acid exchange to glutamate or aspartate at reference position (iv).

In one embodiment of the first aspect, the acyltransferase comprises an amino acid exchange to leucine at reference positions (i) and an amino acid exchange to alanine at reference positions (ii) and (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi).

In one embodiment of the first aspect, the acyltransferase comprises an amino acid exchange to leucine at reference positions (i) and an amino acid exchange to glutamate at reference position (ii) and an amino acid exchange to alanine at reference position (iii); and an amino acid exchange to glutamate or aspartate at reference position (iv).

In one embodiment of the first aspect, the acyltransferase comprises an amino acid exchange to leucine at reference positions (i) and an amino acid exchange to glutamate at reference position (ii) and an amino acid exchange to alanine at reference position (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi).

In one embodiment of the first aspect, the acyltransferase comprises an amino acid exchange to alanine at reference positions (i) and an amino acid exchange to glutamate at reference position (ii) and an amino acid exchange to alanine at reference position (iii); and an amino acid exchange to glutamate or aspartate at reference position (iv).

In one embodiment of the first aspect, the acyltransferase comprises an amino acid exchange to alanine at reference positions (i) and an amino acid exchange to glutamate at reference position (ii) and an amino acid exchange to alanine at reference position (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi).

In one embodiment of the first aspect, the acyltransferase comprises an amino acid exchange to aspartate at reference positions (i) and an amino acid exchange to glutamate at reference position (ii) and an amino acid exchange to alanine at reference position (iii); and an amino acid exchange to glutamate or aspartate at reference position (iv).

In one embodiment of the first aspect, the acyltransferase comprises an amino acid exchange to aspartate at reference positions (i) and an amino acid exchange to glutamate at reference position (ii) and an amino acid exchange to alanine at reference position (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi).

In one embodiment of the first aspect, the acyltransferase comprises an amino acid exchange to aspartate at reference positions (i) and an amino acid exchange to alanine at reference position (ii) and an amino acid exchange to alanine at reference position (iii); and an amino acid exchange to glutamate or aspartate at reference position (iv).

In one embodiment of the first aspect, the acyltransferase comprises an amino acid exchange to aspartate at reference positions (i) and an amino acid exchange to alanine at reference position (ii) and an amino acid exchange to alanine at reference position (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi).

In one embodiment of the first aspect, the acyltransferase comprises an amino acid exchange to alanine at reference positions (i) and an amino acid exchange to glutamate at reference position (ii) and an amino acid exchange to aspartate at reference position (iii); and an amino acid exchange to glutamate or aspartate at reference position (iv), optionally further comprising an amino acid exchange to arginine at reference position (ix).

In one embodiment of the first aspect, the acyltransferase comprises an amino acid exchange to alanine at reference positions (i) and an amino acid exchange to glutamate at reference position (ii) and an amino acid exchange to aspartate at reference position (iii); and an amino acid exchange to aspartate at reference position (iv) and an amino acid exchange to aspartate at reference position (vi) or an amino acid exchange to aspartate at reference position (iv) and an amino acid exchange to glutamate at reference position (vi) or an amino acid exchange to glutamate at reference position (iv) and an amino acid exchange to aspartate at reference position (vi) or an amino acid exchange to glutamate at reference position (iv) and an amino acid exchange to glutamate at reference position (vi), optionally further comprising an amino acid exchange to arginine at reference position (ix).

In some embodiments of the first aspect, the acyltransferase comprises an amino acid exchange to glutamate or aspartate at reference position (iii) and optionally further comprises an amino acid exchange at reference position: (ix) D160 according to SEQ ID NO: 29, or an analogous aspartate or glutamate residue at said reference position as defined in Table 1, wherein the amino acid is exchanged to an amino acid comprising a hydrogen donor residue, preferably arginine, histidine, lysine, glutamine, or asparagine. In some embodiments, the amino acid exchange at reference position (ix) is an exchange to arginine. In other embodiments, the amino acid exchange at reference position (ix) is an exchange to histidine, lysine, glutamine, or asparagine.

In some embodiments of the first aspect, the acyltransferase originates from a fatty acid synthase of an organism selected from vertebrata, including aves and mammalia, including artiodactyla, perissodactyla, carnivora, primates and rodentia, preferably wherein the acyltransferase originates from a fatty acid synthase comprising or consisting of a wild type amino acid sequence selected from any one of SEQ ID NO: 1 to 28, or a sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity thereto.

In some embodiments of the first aspect, the acyltransferase comprises or consists of an amino acid sequence of any of SEQ ID NO: 57 to 82 or 111 to 133, or a sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity thereto.

In a second aspect, there is provided a fusion protein comprising at least one acyltransferase of any one of the embodiments of the first aspect, preferably wherein the fusion protein further comprises at least one linker domain and optionally at least one ketoacyl synthase domain.

In some embodiments, the fusion protein may comprise a tag for purification, for example but not limited to at least one His-tag, step-tag, FLAG-tag, myc-tag, MBP-tag, GST-tag, or a combination thereof.

The acyltransferase of the present invention of preferably linked to other functional PKS or FAS parts and/or domains, in particular a ketoacyl synthase domain, via a FAS and/or PKS LD. Of course, the acyltransferase may also be linked to other functional parts and/or domains by conventional polypeptide linkers known in the art, such as glycine linkers, GS linkers, PT-linkers or other commonly used linkers.

In some embodiments, the fusion protein may be a polyketide synthase (PKS), or a part thereof, in which at least one acyltransferase has been replaced by an acyltransferase of the present invention, wherein the PKS portion of the fusion protein may comprise further engineered modifications.

Type I PKSs are large multifunctional proteins that have a modular enzymatic structure, in which one module comprises a set of functional enzymatic domains involved in the incorporation of an α-carboxyacyl extender unit into a growing polyketide chain. A type I PKS module comprises at least three different functional domains: a ketoacyl synthase (KS), which is responsible for catalyzing the Claisen condensation between an extender unit and the growing polyketide chain; an acyltransferase, which selectively binds an extender unit and trans-esterifies the extender onto an acyl carrier protein (ACP); the ACP serves as the acceptor for the extender unit and the growing polyketide chain. A type I PKS may further comprise additional domains, such as a ketoacyl reductase (KR), which reduces the β-ketone to a hydroxyl group, a dehydratase (DH) which removes the hydroxyl group and creates a double bond between the α and β carbon, an enoyl reductase (ER) which reduces the double bond generated by the DH domain, and a thioesterase (TE) terminating the synthesis process. A type I PKS may be an iterative PKS, catalyzing multiple elongation cycles repeatedly using the same functional domains, or it may be an assembly-line PKS comprising different modules, each of which catalyzes one elongation step (Hertweck 2009, Cogan et al. 2021).

Type II PKS systems comprise discrete proteins, each individually comprising enzymatic activities for polyketide synthesis.

Usually, the starter unit or the extender unit is a CoA-bound acyl group, such as acetyl-CoA, malonyl-CoA, methylmalonyl-CoA or propionyl-CoA, which are selectively recognized and transferred to the ACP by the acyltransferase. By exchanging the acyltransferase to an engineered acyltransferase of the present invention, a PKS system may be reprogrammed to incorporate extender units bound to other coenzyme moieties, including synthetic coenzyme moieties. The acyltransferase of a PKS may be replaced by an acyltransferase of the present invention through regular cloning strategies available to the skilled person.

In modular, assembly-line PKS systems, the acyltransferase of one, or two, or more, or all modules may be replaced by a reprogrammed acyltransferase of the present invention, optionally including the adjacent LD, or a part thereof, and/or KS, to produce a reprogrammed PKS system capable of using extender units bound by coenzyme moieties other than CoA.

An acyltransferase or fusion protein according to the various embodiments of the first or second aspect, may be purified using protein purification methods disclosed herein or any other conventional protein purification methods.

In a third aspect, there is provided a nucleic acid encoding the acyltransferase of the first aspect, or the fusion protein of the second aspect.

The nucleic acid may be double or single stranded DNA or RNA, including mRNA. A nucleic acid that is not part of an expression construct or vector may comprise other sequences and/or modification for improving the stability.

In a fourth aspect, there is provided an expression construct or vector comprising at least one nucleic acid of the third aspect.

The nucleic acid of the third aspect and the expression construct or vector of the fourth aspect may further comprise sequences for expression in a desired host cell, such as a promoter, including inducible promoters, and/or a transcription terminator operably linked to the nucleic acid encoding the acyltransferase or the fusion protein, a selectable marker, a sequence for replication in a desired host cell, and/or further regulatory sequences, including sequences regulatory for transcription, RNA processing and/or stability, and/or translation. The nucleic acid or expression construct or vector may comprise a cDNA sequence, devoid of introns, encoding the acyltransferase and/or fusion protein of the present invention, e.g. for expression in prokaryotic cells. Recombinant protein expression, as well as suitable vectors and expression systems, promoters and further regulatory sequences are well-established for a multitude of cell types. The skilled person is well-aware of the particulars and requirements for recombinant protein expression in different cell types and can easily determine suitable components to choose for expression in the desired host cell. Vector backbones for an expression construct or vector may for example be pET22b, pET300, pET301, pET28b or pGEX-6P-1 vector systems, but are not limited thereto.

In certain embodiments of the third or fourth aspect, the nucleic acid or expression construct or vector is codon optimized, wherein the codon optimization may be adapted according to the desired host cell. Codon optimizations for different cell types are well-established in the art and tools for codon optimization are readily available to the skilled person.

The nucleic acid of the third aspect and/or the expression construct or vector of the fourth aspect may be suitable for stable replication and maintenance in the desired host cell. The nucleic acid and/or expression construct or vector may further be suitable for integration into the host genome.

In a fifth aspect, there is provided a cell comprising at least one acyltransferase of the first aspect; and/or at least one fusion protein of the second aspect; and/or at least one nucleic acid of the third aspect; and/or at least one expression construct or vector of the fourth aspect.

In one embodiment, at least one nucleic acid encoding at least one acyltransferase of the first aspect and/or at least one fusion protein of the second aspect may be stably integrated into the genome, including the chromosomal/nuclear genome and/or the plastid genome, of the host cell, e.g. by any conventional genome editing strategy and/or any conventional vector suitable for genome integration. In certain embodiments, the at least one nucleic acid of the present invention may be integrated into the genome by replacing an endogenous nucleic acid encoding an acyltransferase.

In another embodiment, an endogenous gene of a host cell encoding an acyltransferase is mutated to encode an acyltransferase of the present invention, wherein the mutation may be performed by any mutation strategy, preferably a SDN-1 (site directed nuclease 1) mutation strategy.

In another embodiment, at least one nucleic acid of the third aspect and/or at least one expression construct or vector of the fourth aspect is present extrachromosomally in the host cell.

An acyltransferase of the present invention may be expressed in any cellular expression system, established systems include prokaryotic cells, including Escherichia coli strains, such as but not restricted to various different well-established BL21 or BL21(DE3) expression strains as well as derivatives thereof, including for example Rosetta, BAP1 or NiCo21(DE3) strains, other E. coli strains, such as E. coli K12 strain JM83 or other prokaryotic cells such as Bacillus subtilis expression systems, eukaryotic expression systems, for example including Saccharomyces cerevisiae cells, Komagataella phaffii (formerly called Pichia pastoris) cells, Aspergillus niger cells, or immortalized cell lines, such as insect cell expression systems, for example Sf-9 or Sf-21 cells, or mammalian cell lines, such Chinese hamster Ovary (CHO) cells, HEK 293 cells and the like.

In one embodiment, at least one endogenous acyltransferase of the cell is knocked out, knocked down or otherwise inactivated and complemented with at least one acyltransferase and/or fusion protein of the present invention; and/or at least one nucleic acid, expression construct and/or vector encoding the same.

In preferred embodiments, the cell comprises a PKS system in which at least one acyltransferase has been replaced with an acyltransferase of the present invention. In certain embodiments, the cell comprises a modular type I PKS system in which the acyltransferase of one, or two, or more, or all PKS modules has been replaced by a reprogrammed acyltransferase of the present invention, optionally including the adjacent LD, or a part thereof, and/or KS, of the present invention.

In a sixth aspect, there is provided a kit comprising at least one acyltransferase of the first aspect; and/or at least one fusion protein of the second aspect; and/or at least one nucleic acid of the third aspect; and/or at least one expression construct or vector of the fourth aspect; and/or at least one cell of the fifth aspect; optionally further comprising at least one container; and/or a set of reagents including buffer, medium and/or substrate(s); and/or means of transfection. A kit will usually comprise all agents, buffers, co-factors etc. necessary to guarantee the functionality of the at least one acyltransferase in an assay of interest.

In one embodiment, the kit of the sixth aspect is a kit for in vivo synthesis in at least one cell according to the fifth aspect.

In another embodiment, the kit of the sixth aspect comprises at least one purified or partially purified acyltransferase of the first aspect and/or at least one purified or partially purified fusion protein of the second aspect, at least one buffer, at least one reaction substrate comprising a coenzyme moiety other than CoA and optionally further suitable reaction components.

In another embodiment, the kit of the sixth aspect comprises lysate of at least one cell according to the fifth aspect, at least one reaction substrate comprising a coenzyme moiety other than CoA, at least one buffer and optionally further suitable reaction components.

In another embodiment, the kit of the sixth aspect comprises at least one in vitro transcription/translation system expressing at least one nucleic acid of the third aspect and/or at least one expression construct or vector of the fourth aspect, at least one reaction substrate comprising a coenzyme moiety other than CoA and optionally at least one buffer and/or further suitable reaction components.

In a seventh aspect, there is provided a use of at least one acyltransferase as defined in the first aspect; and/or at least one fusion protein as defined in the second aspect; and/or at least one nucleic acid as defined in the third aspect; and/or at least one expression construct or vector as defined in the fourth aspect; and/or at least one cell as defined in the fifth aspect; and/or at least one kit as defined in the sixth aspect; for polyketide synthesis, fatty acid synthesis and/or non-ribosomal peptide synthesis, optionally wherein it is a use for reacting at least two different substrates.

As a Selectase according to the first aspect comprises specifically reprogrammed coenzyme-binding properties without the necessity to alter amino acid residues involved in the acyl moiety binding, the acyltransferase of the present invention can retain the natural polyspecificity regarding the acyl moiety. Retaining the acyl polyspecificity can be of great importance, e.g. for using one acyltransferase for different acyl-moieties as substrates, in particular when using at least two different substrates for polyketide synthesis, fatty acid synthesis and/or non-ribosomal peptide synthesis.

“Polyketide synthesis” as used herein includes synthesis of diketides and oligoketides.

In certain embodiments of the seventh or ninth aspect, the polyketide synthesis, fatty acid synthesis and/or non-ribosomal peptide synthesis may be a synthesis of polyketide, and/or fatty acid and/or protein hybrids with metabolites of other biosynthetic synthesis pathways.

In one embodiment, synthesis of the seventh aspect is in vivo synthesis in at least one cell according to the fifth aspect, further using at least one suitable medium and at least one reaction substrate comprising a coenzyme moiety other than CoA, the at least one cell thus representing an engineered production strain.

In another embodiment, synthesis of the seventh aspect is in vitro synthesis using at least one purified or partially purified acyltransferase of the first aspect and/or at least one purified or partially purified fusion protein of the second aspect, at least one buffer, at least one reaction substrate comprising a coenzyme moiety other than CoA and optionally further suitable reaction components.

In another embodiment, synthesis of the seventh aspect is in vitro synthesis using lysate of at least one cell according to the fifth aspect, at least one reaction substrate comprising a coenzyme moiety other than CoA, at least one buffer and optionally further suitable reaction components.

In another embodiment, synthesis of the seventh aspect is in vitro synthesis using at least one in vitro transcription/translation system expressing at least one nucleic acid of the third aspect and/or at least one expression construct or vector of the fourth aspect, at least one reaction substrate comprising a coenzyme moiety other than CoA and optionally at least one buffer and/or further suitable reaction components.

In an eighth aspect there is provided a method comprising: (a) providing (i) 3-Oxo-3-[[2-[(1-oxohexyl)amino]ethyl]thio]-propanoic acid; and (iia) at least one acyltransferase as defined in the first aspect; and/or at least one fusion protein as defined in the second aspect; and/or at least one nucleic acid as defined in the third aspect; and/or at least one expression construct or vector as defined in the fourth aspect; and at least one acyl carrier protein (ACP), or a nucleic acid encoding the same; or (iib) at least one cell as defined in the fifth aspect, further comprising at least one ACP; and (b) allowing the at least one acyltransferase to transfer at least one malonyl-moiety from the 3-Oxo-3-[[2-[(1-oxohexyl)amino]ethyl]thio]-propanoic acid to the at least one ACP; (c) optionally obtaining at least one acyl-ACP.

In a ninth aspect there is provided a use of 3-Oxo-3-[[2-[(1-oxohexyl)amino]ethyl]thio]-propanoic acid for polyketide synthesis, fatty acid synthesis and/or non-ribosomal peptide synthesis.

In one embodiment, synthesis of the ninth aspect is in vivo synthesis in at least one cell according to the fifth aspect, further using at least one suitable medium and at least one reaction substrate comprising the 3-Oxo-3-[[2-[(1-oxohexyl)amino]ethyl]thio]-propanoic acid, the at least one cell thus representing an engineered production strain.

In another embodiment, synthesis of the ninth aspect is in vitro synthesis further using at least one purified or partially purified acyltransferase of the first aspect and/or at least one purified or partially fusion protein of the second aspect, at least one buffer and optionally further suitable reaction components.

In another embodiment, synthesis of the ninth aspect is in vitro synthesis further using lysate of at least one cell according to the fifth aspect, at least one buffer and optionally further suitable reaction components.

In another embodiment, synthesis of the ninth aspect is in vitro synthesis further using at least one in vitro transcription/translation system expressing at least one nucleic acid of the third aspect and/or at least one expression construct or vector of the fourth aspect, and optionally at least one buffer and/or further suitable reaction components.

### EXAMPLES

### Example 1: In Silico Design for Targeted Acyltransferase Reprogramming

Due to their modular nature and their innate variability in acyl backbone reduction, PKS systems are an enticing enzymatic platform to be exploited for synthesizing a plethora of different useful biomolecules, in particular for drug design. A crucial aspect limiting a versatile and effective biotechnological use of PKS is the nature of naturally occurring acyltransferases, which have a relatively high specificity when it comes to the extender subunits (Smith and Tsai 2007). The MAT domains of metazoan FAS enzymes were shown to allow an efficient transfer of different acyl substrates (Rittner et al. 2018).

However, while a FAS MAT might therefore offer a certain versatility regarding the acyl group of the substrate, both FAS MATs and PKS ATs suffer from a crucial shortcoming: they are fully dependent on CoA-bound substrates. The dependency on Acyl-CoA substrates strongly limited the possibility for artificial biomolecule production using an engineered PKS system for in vivo synthesis. CoA-bound molecules, in particular Acetyl-CoA and Malonyl-CoA, are highly abundant and unavoidable in any in vivo setting, thereby interfering with any possible biosynthesis based on CoA-bound substrates. While the MAT crystal structure is solved (Rittner et al. 2020), it has not been envisioned that the crucial activating component of CoA may be replaced, let alone how an acyltransferase may be artificially reprogrammed so that it does not use Acyl-CoA substrates and, instead, specifically accepts subunits bound to different coenzyme moieties, such as synthetic coenzyme moieties.

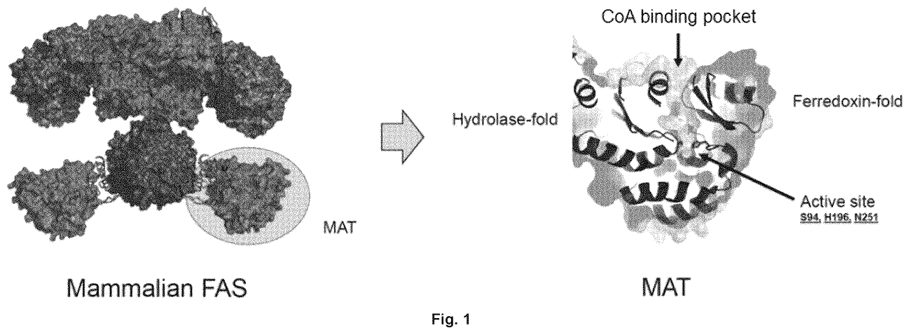

Based on the crystal structure and the position of the CoA binding pocket therein (see FIG. 1), it was speculated that target residues that are highly attractive to reprogramming a natural acyltransferase could be identified. However, mutation experiments showed that amino acid exchanges at certain residues do not allow any functional protein expression. Therefore, an iterative testing strategy and protein engineering techniques combined with in silico searches were applied to define acyltransferase mutants that can be manipulated in a way to efficiently block CoA binding and—at the same time—that allow binding of different coenzyme moieties, while still allowing correct overall folding of the protein. Moreover, to offer a high versatility including the choice of acyltransferase protein itself, alignments of FAS amino acid sequences from various genera were performed in addition to the structural analysis. An alignment of 28 different example sequences is shown in Table 1. After extensive analysis, eight different target residues have been identified (see FIG. 2) in those sequences showing a certain degree of identity. To this end, various hot spots for mutational activities were defined that were tested in the following as further detailed below.

TABLE 1

| | |
|---|---|
| | Reference positions |
| | (viii) (ix)(i) |
| SEQ 1 Mus musculus | 621 AAVGLSWEECKQRCPAGVVPACHNSEDTVT |
| SEQ 2 Rattus norvegicus | 621 AAVGLSWEECKQRCPPGVVPACHNSEDTVT |
| SEQ 3 Cricetulus griseus | 621 AAVGLSWEECKQRCPSGVVPACHNSEDTVT |
| SEQ 4 Homo sapiens | 621 AAVGLSWEECKQRCPPGVVPACHNSKDTVT |
| SEQ 5 Macaca mulatta | 621 AAVGLSWEECKQRCPPGVVPACHNSKDTVT |
| SEQ 6 Bos taurus | 621 AAVGLTWEECKQRCPPGIVPACHNCIDTVT |
| SEQ 7 Capra hircus | 620 AAVGLTWEECKQRCPPGIVPACHNSIDTVT |
| SEQ 8 Equus caballus | 621 AAVGLSWEECKQRCPPGVVPACHNSKDTVT |
| SEQ 9 Felis catus | 761 AAVGLSWEECKQRCPPGVVPACHNSEDTVT |
| SEQ 10 Sus scrofa | 621 AAVGLSWEECKQRCPPGIVPACHNSKDTVT |
| SEQ 11 Cuculus canorus | 615 AAVGLSWEECKQICPPNVVPACHNSEDTVT |
| SEQ 12 Calypte anna | 622 AAVGLTWEECKQQCPPNVVPACHNSEDTVT |
| SEQ 13 Anas platyrhynchos | 620 AAVGLTWEECKQRCPPNVVPACHNSEDTVT |
| SEQ 14 Gallus gallus | 620 AAVGLTWEECKQRCPPNVVPACHNSEDTVT |
| SEQ 15 Nestor notabilis | 615 AAVGLTWEECKQRCPPNVVPACHNSEDTVT |
| SEQ 16 Podiceps cristatus | 622 AAVGLTWEECKQCCPPNVVPACHNSEDTVT |
| SEQ 17 Balearica regulorum gibbericeps | 624 AAVGLTWEECKQCCPPNVVPACHNSEDTVT |
| SEQ 18 Phaethon lepturus | 621 AAVGLTWEECKQCCPPNVVPACHNSEDTVT |
| SEQ 19 Phoenicopterus ruber | 246 AAVGLTWEECKQCCPPNVVPACHNSEDTVT |
| SEQ 20 Ooceraea biroi | 650 AAVGLSWEDAQKMCPPNVSPACHNSMDSVT |
| SEQ 21 Anopheles funestus | 669 AAVGLSWEECKQKLPKDVIPACHNSADSVT |
| SEQ 22 Anopheles darlingi | 677 AAVGLSWEDCKQKLPKDVIPACHNSSDSVT |
| SEQ 23 Glossina austeni | 780 AAVGLSWEEAHKRLPADCFPACHNSAENCT |
| SEQ 24 Drosophila melanogaster | 761 AAVGLSWEDAHSRVPSDCFPVCHNSEDNCT |
| SEQ 25 Brugia malayi | 633 AAVGLSWEEAGKRCPEGVIPACHNAADSVT |
| SEQ 26 Caenorhabditis elegans | 661 AAVGLTWEQVKEQAPPGVVAACHNGADSVT |
| SEQ 27 Caenorhabditis briggsae | 778 AAVGLTWEEVKAQAPPGVVAACHNGADSVT |
| SEQ 28 Caenorhabditis remanei | 634 AAVGLTWEEVKAQAPKGVVAACHNGADSVT |

```
                                            Reference positions
                                                    (ii) (iv)
SEQ 1 ISGPQAAVNEFVEQLKQEGVFAKEVRTGGLAFHSYFMEGIAPTLLQALKKVIREPRPRSARWLSTS
SEQ 2 ISGPQAAVNEFVEQLKQEGVFAKEVRTGGLAFHSYFMEGIAPTLLQALKKVIREPRPRSARWLSTS
SEQ 3 ISGPQAAVSEFVEQLKQEGVFAKEVRTGGLAFHSYFMEGIAPMLLQALKKVIREPRPRSARWLSTS
SEQ 4 ISGPQAPVFEFVEQLRKEGVFAKEVRTGGMAFHSYFMEAIAPPLLQELKKVIREPKPRSARWLSTS
SEQ 5 ISGPQASVFEFMEQLRKEGVFAKEVRTGGMAFHSYFMEAIAPPLLQALKKVIREPKPRSARWLSTS
SEQ 6 ISGPQASMLEFVQQLKQEGVFAKEVRTGGMAFHSYFMDAIAPMLLQQLKKVIREPQPRSPRWLSTS
SEQ 7 ISGPQASMLEFVKQLKQEGVFAKEVQTGGMAFHSYFMDAIAPTLLQQLKKVIREPQLRSPRWLSTS
SEQ 8 ISGPQAAVSEFVEQLKQEGVFAKEVRIGGLAFHSYFMDSISHTLLQALKKVIREPRPRSVRWLSTS
SEQ 9 ISGPQAEVAAFVAELKREGVFAKEVRTGGMAFHSYFMDSIAPTLLQALKKVIREPRPRSARWLSTS
SEQ 10 ISGPQAAMSEFLQQLKREDVFVKEVRIGGIAFHSYFMESIAPTLLRQLRKVILDPKPRSKRWLSTS
SEQ 11 VSGPLGAVSEFVAKLKKDGVFAKEVRISGVAFHSHYMASIAPVLLSALKKVIPHPKPRSPRWISTS
SEQ 12 ISGPLATVSEFVAKLKKAGVFAKEVRSAGVAFHSHYMASIAPVLLSALKKVIPHPKPRSARWISTS
SEQ 13 ISGPLDSVNEFVAKLKKDGVFAKEVRSAGVAFHSYYMASIAPALLSALRKVIPHPKPRSARWISTS
SEQ 14 VSGPLDSVSEFVTKLKKDGVFAKEVRRAGVAFHSYYMASIAPALLSALKKVIPHPKPRSARWISTS
SEQ 15 VSGPLESVTEFVAKLKKDGVFAKEVRSAGVAYHSHYMASIAPVLLNALKKVIPHPKPRSARWISTS
SEQ 16 ISGPLDSVHEFVAKLKKDGVFAKEVRSAGVAFHSYYMASIAPVLLSALKKVIPHPKPRSARWISTS
SEQ 17 VSGPLDSVNEFVAKLKKDGVFAKEVRSAGVAFHSYYMASIAPVLLSALKKVIPHPKPRSARWISTS
SEQ 18 VSGPLDSVNEFVAKLKKDGVFAKEVRSAGVAFHSYYMASIAPVLLSALKKVIPHPKPRSARWISTS
SEQ 19 VSGPLDSVREFVAKLKKDGVFAKEVRSAGVAFHSYYMASIAPVLLSALKKVIPHPKPRSARWISTS
SEQ 20 ISGPTKDVLKFVEELKSKNIFAKLVRSSGIAFHSKYIASAGPKLRASLDKIIPNPKQRSAKWISSS
SEQ 21 ISGPVNSVGKVIADLNAQGIFAKGVKSSGIAFHSRYIADAAPKLRKSLDKIIPNPKNRTPRWISTS
SEQ 22 ISGPVASVGKVIADLNAQGIFAKGVKSSGIAFHSRYIADAAPKLRKSLDKIIPNPKPRTQRWISTS
SEQ 23 ISGPEESIDAVCQKLTAEGVFARAVKSSGYAFHSKYIADAGPKLRKSLEKVIPNAKNRSPRWISSS
SEQ 24 ISGPEASIEALVAKLNAEGVFAKAVNSSGYAFHSKYIAEAGPKLRKSLEKIIPNAKNRTARWISTS
SEQ 25 ISGDAEKIKEFVEELKKEDIFGKLVDSSGIPFHSPAMLKVKDKMLKAMRTSVPNPKPRSSRWISTS
SEQ 26 ISGDAEGVATFCAQLKEKDIFAKVVDTSGIPFHSPAMLAVQDEMIECMRTAVPEPKPRSSKWISTS
SEQ 27 ISGDAEGVASFCAQLKEKEIFAKVVDTSGIPFHSPAMLAVKDEMIESMRTAVPEPKPRSSKWISTS
SEQ 28 ISGDAEGVATFCAQLKEKDIFAKVVDTSGIPFHSPAMLAVKDEMIESMRTAVPEPKPRSSKWISTS
                                                                                                            Reference positions
                                                                                                                  (vii) (v)(iii)
SEQ 1 IPEAQWQSSLARISSAEYNVNNLVSPVLFQEALWHIPEHAVVLEIAPHALLQAVLKRGVKSSCTII
SEQ 2 IPEAQWQSSLARISSAEYNVNNLVSPVLFQEALWHVPEHAVVLEIAPHALLQAVLKRGVKPSCTII
SEQ 3 IPEAQWQSSLARISSAEYNVNNLVSPVLFQEALWHVPEHAVLLEIAPHALLQAVLKRGVKSSCTII
SEQ 4 IPEAQWHSSLARISSAEYNVNNLVSPVLFQEALWHVPEHAVVLEIAPHALLQAVLKRGLKPSCTII
SEQ 5 IPEAQWHSSLARISSAEYNVNNLVSPVLFQEALCHVPEHAVVLEVAPHALLQAVLKRGLKPGCTII
SEQ 6 IPETQWQESLARTFSAEYNVNNLVSPVLFQEALWRVPEDAVVLEIAPHALLQAVLKRGLKSSCTII
SEQ 7 IPESQWHESLARTFSAEYNVNNLVSPVLFQEALWHVPENAVVLEIAPHALLQAILKRGLQPSCTII
SEQ 8 IPEAQWQSSLARTFSAEYNVNNLVSPVLFQEALCHVPEHAVVLEIAPHALLQAVLKRGLKPSCTIV
SEQ 9 IPEAQWQGSLARTFSAEYNVNNLVSPVLFQEALWHVPGDAVVLEIAPHALLQAVLKRGLKSSCTIV
SEQ 10 IPEAQWQGSLARTFSAEYSVNNLVSPVLFQEALQHVPAHAVVVEIAPHALLQAVLKRSLESSCTII
SEQ 11 IPESQWQSDLARYSSAEYHVNNLVNPVLFHEGLSHVPGNALVVEIAPHALLQAILKRSLKPTCIIL
SEQ 12 IPESQWQSDLAKYSSAEYHVNNLVSPVLFHEGLKHIPENAVVVEIAPHALLQAILKRTLKPTCTIL
SEQ 13 IPESQWQSDLARNSSAEYYVNNLVSPVLFHEGLKHIPENAVVVEIAPHALLQAILRRSLKPSCTIL
SEQ 14 IPESQWQSDLARNSSAEYHVNNLVNPVLFHEGLKHIPENAVVVEIAPHALLQAILRRTLKPTCTIL
SEQ 15 IPESQWQSDLARSCSAEYLVNNLVNPVLFHDGLKHVPENAVVVEIAPHALLQAILRRTLKPTCTIL
SEQ 16 IPESQWQSDLARNSSAEYHVNNLVNPVLFHEGLKHVPENAVVVEIAPHALLQAILRRTLKPTCTIL
SEQ 17 IPESQWQSDLARNSSAEYHVNNLVNPVLFHEGLKHVPENAVVVEIAPHALLQAILRRTLKPTCTIL
SEQ 18 IPESQWQSDLARNSSAEYHVNNLVNPVLFHEGLKHIPENAVVVEIAPHALLQAILRRILKPTCTIL
SEQ 19 IPESQWQSDLARNSSAEYHVNNLVNPVLFQEGLKHIPENAVVVEIAPHALLQAILRRALKPTCTIL
SEQ 20 IPEAAWGSPLAQVSSSAYHVNNLLSPVLFQEAIAHIPDNAITIEIAPHCLLQAILRRSLPSTVINV
SEQ 21 IPEESWPTPLAQQSSSAYHVNNLLSPVLFSEGLKHVPANAICIEIAPHGLLQAILKRALGKDATNL
SEQ 22 IPEESWGTALAQQSSSAYHVNNLLSPVLFAEGLKHVPANAICIEIAPHGLLQAILKRALGKEATNL
SEQ 23 IPEVAWNTAIAQQASAAYHVNNLLSPVLFHQALQHVPKNAICIEVAPTGLLQAILKRSLGNETTNL
SEQ 24 IPESAWNTPVAKQSSAAYHVNNLLSPVLFHEALQHVPKNAISVEIAPHGLLQAILKRALGPDATNL
SEQ 25 IPESNWENELAQMCSADYHINNAVSPVLFYEALQKIPANAVTIEIAPHCLMHSILRRSLQKTCTNV
SEQ 26 IPEDDWESDLAATCSAEYHVHNACSPVLFYEAIQKIPANAVTIEMAPHSLMQAILRRSLQKTVINV
SEQ 27 IPEEDWESDLAATCSAEYHVHNACSPVLFYEALQKIPANAVTIEMAPHSLMQAILRRSLQKTVINV
SEQ 28 IPEEDWESDLAATCSAEYHVHNACSPVLFYEALQKIPANAVTIEMAPHSLMQAILRRSLMKTVINV
          Reference position
                      (vi)
SEQ 1 PLM-KR---DHKDNLEFFLTNLGKVHLTGINVNPNALFPPVEFP--APRGTPLISPHI 834
SEQ 2 PLM-KR---DHKDNLEFFLTNLGKVHLTGIDINPNALFPPVEFP--VPRGTPLISPHI 834
SEQ 3 PLM-KR---DHKDNLEFFLTNLGKVHLTGIDVNPNALFPPVDFP--APRGTPLISPHI 834
SEQ 4 PLM-KK---DHRDNLEFFLAGIGRLHLSGIDANPNALFPPVEFP--APRGTPLISPLI 834
SEQ 5 PLM-KK---DHRDNLEFFLIGIGRLHLSGIDANPNALFPPVEFP--APRGTPLISPLI 834
SEQ 6 PLM-KK---DHRDNLEFFLSNVGQLYLTGIDVNPNGLFPPVEFP--APRGTPLISPHI 834
SEQ 7 PLM-KK---DHRDNLEFFLSNVGQLYLTGIDVNPNGLFPPVEFP--APRGTPLISPHI 833
SEQ 8 PLM-KK---DHRDNLEFFLSNVGRLHLMGIDVNPNGLEPPVEFP--APRGTPLISPHI 834
SEQ 9 PLM-KK---DQRDNLEFFLSNVGKLHLLGEDVNPNGLLPPVEFP--VPRGTPLISPHI 974
SEQ 10 PLM-KK---DHRDNLEFFLSNVGRLHLAGVSVNPNGLFPPVEFP--APRGTPLISPHX 834
SEQ 11 PLM-KK---EHKNNLEFFLTQLGKFHMSGVNENGNNLFPRVEYP--VPVETPLISPHI 828
SEQ 12 PLM-KK---DHKNNLEFELTQAGKIHLIGINVLGNNLFPPVEYP--VPVGTPLISPYI 835
SEQ 13 PLM-KK---DQKNNLEFFLTQAGKIHLTGINVLGNNMFPSVEYP--VPVGTPLISPYI 833
SEQ 14 PLM-KK---DHKNNLEFFLTQTGKIHLTGINVLGNNLFPPVEYP--VPVGTPLISPYI 833
SEQ 15 PLM-KK---DQKNNLEFFLTQTGKIHLTGINVLGNNLFPLVEYP--VPVGTPLISPYI 828
SEQ 16 PLM-KK---DHKNNLEFFLTQTGKIHLTGINVLGNNLLPLVEYP--VPVGTPLISPYI 835
SEQ 17 PLM-KK---EHKNNLEFELTQTGKIHLTGINVLGNNLFPLVEYP--VPVGTPLISPYI 837
SEQ 18 PLM-KK---EHKNNLEFELTQTGKIHLTGINVLGNNLFPLVEYP--VPVGTPLISPYI 834
SEQ 19 PLM-KK---EHKNNLEFFLTQTGKIHLTGINVLGNNLFPLVEYP--VPVGTPLISPYI 459
SEQ 20 SLH-MR---DHTNNLAFLLSNIGKLYMAGAQPNISKLYPPVSFP--VGRGTPMIGPLV 863
SEQ 21 SLM-KR---DHANNMIFLLSNLGKLYAAGVQPQVQKLYRPITYP--VGRGTPMLNSLV 882
SEQ 22 SLM-KR---DHDNNLIFLLSNLGKLYAAGAQPQVQKLYPPITYP--VGRGTPMLNSLV 890
SEQ 23 SLI-KR---DYENNPEFFLASIGKLYAAGAQPQIMTLSKPISYP--VGRGTPMLGCKV 993
SEQ 24 SLV-KR---GHENNVEFFLINVGKLFAAGAQPQVLTLVRPISYP--VGRGTPMLNSKV 974
SEQ 25 GLINMK---EKDRELESFLQALGKIYQTGITIHIEALYPAIQYP--VPIGTPMISPMW 847
SEQ 26 GLM-NRPKSENDDELESELGSLGKIYQAGVNIQITELYPGGQYKGVVPKGTPMIGPMW 879
SEQ 27 GLM-NKPKSENDNELEGELGSLGKIYQAGVNIQISELYPGGQYKGVVPKGTPMIGPMW 996
SEQ 28 GLM-NKPKSENDDELESELGSLGKIYQAGVNIQISELYPGGQYKGVVPKGTPMIGPMW 852
```

### Example 2: Cloning and Expression of Engineered Acyltransferases

The basic plasmid encoding engineered murine acyltransferases was prepared from a synthetic DNA fragment, containing a KS-LD-MAT fragment including Strep- and His-tags and was cloned into a pET22b vector using the restriction enzymes NdeI and XhoI, or with the in-Fusion Snap Assembly Master Mix (Takara). Mutations in the eight different positions corresponding to the reference positions according to the present invention were introduced by PCR using primers harboring the nucleotide variations. The plasmid encoding for the ACP was prepared similarly. An additional ribosome binding site (RBS) and another gene encoding for the 4′-phosphopantetheinyl transferase Sfp was integrated 50 into the same plasmid. The synthetic gene for the ACP was ordered with its native DNA sequence and codon optimized for E. coli.

The PCR mixtures were treated with Dpn1 prior to DNA purification using electrophoresis. DNA was stained and illuminated with Sybr Green DNA Dye (Carl Roth). Appropriate fragments were isolated with a scalpel and DNA was purified with the GeneJet Gel Extraction kit (ThermoFisher). The fragments were transferred to Stellar™ Competent Cells (Takara) chemical competent cells for ligation and amplification of the targeted plasmids. The plasmids were isolated from cells using a standard commercial plasmid purification kit.

The plasmids encoding the MAT or engineered acyltransferases were transformed into BL21(DE3) chemical competent cells (Agilent). Transformed cells were directly transferred to a pre-culture of 5 mL LB-medium containing ampicillin as a selection marker, which was grown over night at 37° C. in an incubation shaker. The pre-culture was used to inoculate a 100-250 mL main-culture in 2×YT or TB medium in a 500 mL Erlenmeyer flask. The culture was grown for 2 hours at 37° C. and then the expression system was induced by adding 250 μM IPTG and was grown for another 16 hours at 20° C. The cells were lysed enzymatically in lysis buffer or centrifuged, re-suspended in lysis buffer containing DNasel and lysed by sonification (Hielscher UP200t, 43% amplitude, 5 minutes, on/off time 20 seconds). After centrifugation, the soluble fraction was purified by Ni-NTA (ThermoFisher) and Strep affinity chromatography (strep-tactin sepharose, IBA Lifesciences GmbH) following the standard protocol. The ACP was purified by size-exclusion chromatography (ROTI Dex-25, carl-Roth) instead of Strep affinity chromatography as a second chromatographic purification. Purified proteins were frozen in aliquots in liquid nitrogen and stored at −25° C. or −80° C. Protein concentrations were determined by their absorbance at 280 nm. The absorbance was recorded on a NanoDrop Onec (ThermoFisher) and converted to concentration using the extinction coefficient of the respective protein, calculated from their primary sequence without the N-terminal methionine in Benchling.

### Example 3: Kinetic Analysis of Engineered Acyltransferases Using MMal-CoA

An enzyme-coupled assay was performed to analyze the transfer of acyl-moieties from CoA to the ACP. Four solutions were prepared as 4× concentrated stocks in the assay buffer. Solution 1 contained the acyltransferase at the concentration range of 0.01 μM-10 μM, depending on the transfer kinetics to achieve good signal to noise ratios. Solution 2 contained the enzyme-coupled system including 8 mM α-ketoglutaric acid, 1.6 mM NAD+, 1.6 mM TPP and 60 mU/100 μL αKGDH. Solution 3 contained the methylmalonyl (MMal)-CoA at concentrations between 0.1 μM and 1,000 μM and solution 4 contained the ACP from murine FAS as a standalone protein at 240 μM. 5 μL of Solutions 1-3 were pipetted into a 384-well Small Volume HiBase Microplates (Greiner Bio-one) and mixed manually. The reactions were started by adding solution 4 with the dispenser system of the microplate reader (ClarioStar, BMG labtech). The reaction progress was recorded using the following settings: 348-20 nm; emission: 476-20 nm; gain: 1500; focal height: 11.9 mm; flashes: 17; orbital averaging: off.

Kinetic data for exemplary mutants is shown in Table 2.

TABLE 2

| Mutant | SEQ ID | Mutation | Kₘ [μM] | k_{cat} [s⁻¹] | k_{cat}/Kₘ [M⁻¹s⁻¹] |
|---|---|---|---|---|---|
| (WT) | 29* | none | 0.62 | 4.000 | 6.5E+06 |
| (WT) | 29 | none | 0.53 | 1.297 | 2.5E+06 |
| A01 | 57 | T161A | 1.47 | 0.051 | 3.5E+04 |
| A02 | 58 | T161Y | 38.78 | 0.428 | 1.1E+04 |
| A03 | 59 | T161A F184A R286A | 3.41 | 0.021 | 6.2E+03 |
| A04 | 60 | T161A F184A K186A R286A | 7.24 | 0.013 | 1.8E+03 |
| A05 | 61 | K186E | 14.16 | 0.101 | 7.2E+03 |
| A06 | — | A282W | Not determined | | |
| A07 | 62 | K186D | 18.63 | 0.093 | 5.0E+03 |
| A08 | 63 | K285D | 0.11 | 0.133 | 1.2E+06 |
| A10 | 65 | R300D | 5.06 | 0.582 | 1.2E+05 |
| A11 | 66 | R300E | 3.33 | 0.834 | 2.5E+05 |
| A12 | 67 | K186D R300D | 66.23 | 0.019 | 2.9E+02 |
| A13 | 68 | K186D R300E | 685.98 | 0.190 | 2.8E+02 |
| A14 | 69 | K186E R300D | 218.27 | 0.051 | 2.3E+02 |
| A15 | 70 | K186E R300E | 58.39 | 0.030 | 5.2E+02 |

Listed SEQ ID NOs refer to corresponding acyltransferase sequences.
Mutations are indicated in reference to the wild type MAT sequence SEQ ID NO: 29.
*Comparative data from Rittner et al. 2018

### Example 4: Analysis of Engineered Acyltransferases Using Mal-CoA

Further engineered mutants were expressed, purified and analyzed, building up on the previous studies. A screen for fifty different exemplary mutants was performed analyzing the transfer of malonyl-moieties from CoA to N-(2-mercaptoethyl)-hexanamide. Four solutions were prepared as 4× concentrated stocks in the assay buffer. Solution 1 contained the acyltransferases at the concentration range of 0.01 μM-10 μM and were arranged in four groups depending on their individual turnover rates to achieve good signal to noise ratios. Solution 2 contained the enzyme-coupled system including 8 mM α-ketoglutaric acid, 1.6 mM NAD+, 1.6 mM TPP and 60 mU/100 μL αKGDH. Solution 3 contained a fixed concentration of malonyl (Mal)-CoA at 200 μM and solution 4 contained the acceptor N-(2-mercaptoethyl)-hexanamide at 200 μM. 5 μL of Solutions 1-3 were pipetted into a 384-well Small Volume HiBase Microplates (Greiner Bio-one) and mixed manually. The reactions were started by adding 5 μL of solution 4 with an automatic multichannel pipette (ClipTip). The reaction progress was recorded using the following settings: 348-20 nm; emission: 476-20 nm; gain: 1700; focal height: 12.1 mm; flashes: 10; orbital averaging: off.

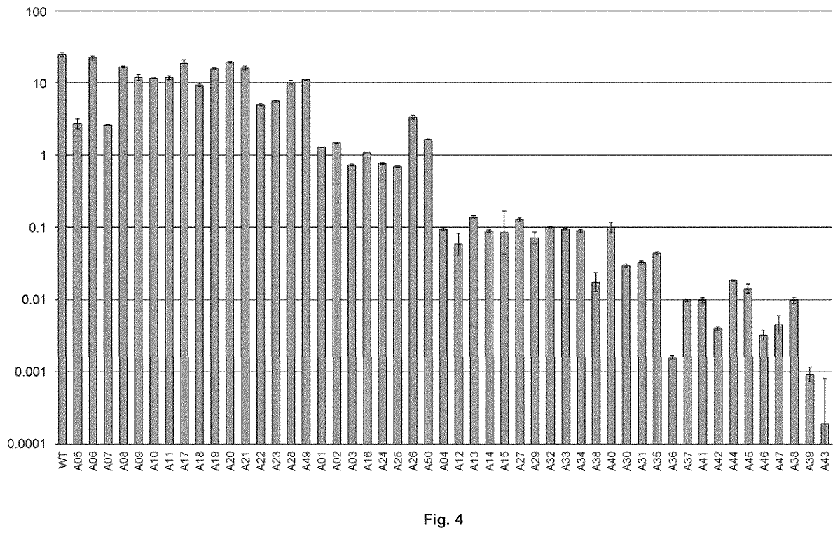

Specific turnover rates of all tested engineered mutants are shown in FIG. 4 plotted on a logarithmic scale. The unit RFU per second was transformed into a turnover per second using known enzyme concentrations and a calibration curve with NADH.

TABLE 3

| Mutant | Mutation | SEQ ID |
|---|---|---|
| A09 | K285E | 64 |
| A16 | A282V | 71 |
| A17 | A282L | 72 |
| A18 | A137V | 73 |
| A19 | A137L | 74 |
| A20 | T161V | 75 |
| A21 | T161L | 76 |
| A22 | T161W | 77 |
| A23 | T161F | 78 |
| A24 | T161D | 79 |
| A25 | T161E | 80 |
| A26 | F184E | 81 |
| A27 | T161D F184E | 82 |
| A28 | T161L F184 R286L | 111 |
| A29 | T161A F184A R286A K186D | 112 |
| A30 | T161A F184A R286A K186E | 113 |
| A31 | T161L F184A R286A K186D | 114 |
| A32 | T161A F184A R286A K186D R300D | 115 |
| A33 | T161A F184A R286A K186E R300D | 116 |
| A34 | T161L F184L R286L K186D | 117 |
| A35 | T161L F184L R286L K186D R300D | 118 |
| A36 | T161D F184A R286A K186D | 119 |
| A37 | T161D F184A R286A K186E | 120 |
| A38 | T161A F184E R286A K186D | 121 |
| A39 | T161A F184E R286A K186E | 122 |
| A40 | T161L F184E R286A K186D | 123 |
| A41 | T161L F184E R286A K186E | 124 |
| A42 | T161L F184E R286A K186D R300D | 125 |
| A43 | T161L F184E R286A K186E R300D | 126 |
| A44 | T161D F184E R286A K186D | 127 |
| A45 | T161D F184E R286A K186E | 128 |
| A46 | T161D F184E R286A K186D R300D | 129 |
| A47 | T161D F184E R286A K186E R300D | 130 |
| A48 | T161A F184E R286D K186D D160R | 131 |
| A49 | R286A | 132 |
| A50 | R286L | 133 |

### Example 5: Validation of Re-Engineered Mutant Activity

To verify the re-engineered mutant activity, having a substrate specificity altered from natural CoA substrates to non-CoA substrates, an alternative method to quantify specific turnover rates for non-CoA substrates was established. Such non-CoA substrates are not detectable by the enzyme-coupled assay. We used a thiol-sensitive fluorophore (ThioGlo4; Yi et al. 2009) to quantify the released free thiol groups from the substrate 3-Oxo-3-[[2-[(1-oxohexyl)amino]ethyl]thio]-propanoic acid after the enzyme-specific transfer of the malonyl moiety to a non-thiol acceptor. Briefly, three solutions were prepared in either a twofold concentration or as 4× concentrated stocks in the assay buffer. Solution 1 contained the acyltransferases at concentrations between 50-500 nM depending on their purified yields and were diluted twofold in the assay. Solution 2 contained a fixed concentration of the substrate 3-Oxo-3-[[2-[(1-oxohexyl)amino]ethyl]thio]-propanoic acid at 200 μM and solution 3 contained the acceptor N-(2-hydroxyethyl)-hexanamide at 20 mM. 10 μL of Solutions 1 and 5 μL of Solution 2 were pipetted into a 384-well Small Volume HiBase Microplates (Greiner Bio-one). The reactions were started by adding 5 μL of solution 4 with a multichannel pipette (ClipTip) and incubated for 10 min at RT. The reactions were stopped by adding 2.5 μL 3 M HCl, neutralized by adding 2.5 μL 4 M NaOH and 10 μL of the reaction mixture was transferred to new wells. Free thiols were detected by adding 10 μL of 120 μM ThioGlo in the buffer 250 mM potassium phosphate, 1 mM EDTA and 10% glycerol, mixing and fluorescence detection at the microplate reader (ClarioStar, BMG labtech) using the following settings: 400-20 nm; emission: 465-20 nm; gain: 700; focal height: 12.5 mm; flashes: 20; orbital averaging: off. Wells not containing any enzyme, but the substrate and acceptor in the mentioned concentrations served as the background and reflect non-enzyme specific transacylation.

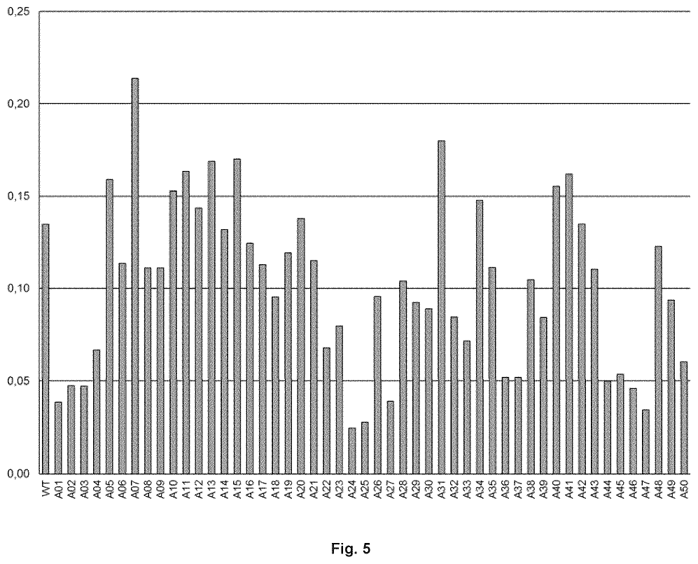

Results of the malonyl transfer from the non-CoA substrate are shown in FIG. 5.

### Example 6: In-Depth Kinetic Analysis

In-depth kinetic analysis results allowing a comparison of enzyme kinetic parameters for the transfer of malonyl-moieties from either Mal-CoA or 3-Oxo-3-[[2-[(1-oxohexyl)amino]ethyl]thio]-propanoic acid to the ACP are shown for a representative mutant (A35) herein. Enzyme kinetics were determined with the established enzyme-coupled assay for Mal-CoA kinetics and a variation of the new thiol quantifying assay using ThioGlo.

Enzyme-coupled assay was performed as described in Example 3 with 4× concentrated stocks. The enzyme concentration of A35 in Solution 1 was 4.2 μM, the substrate concentrations of Mal-CoA in Solutions 3 were between 8-400 μM and the acceptor concentration of ACP in Solution 4 was 340 μM.

A variation of the thiol quantification assay in Example 5 was established to be able to separate the liberated thiol groups from 3-Oxo-3-[[2-[(1-oxohexyl)amino]ethyl]thio]-propanoic acid after acyl-transfer from the thiol groups of the acceptor ACP. The produced N-(2-mercaptoethyl)-hexanamide was extracted from the reaction wells by chloroform extraction. Hence, the assay was performed as described in example 5, but after stopping the reactions with 3 μL HCl, every well was extracted with 2×100 μL chloroform. First 50 μL of the lower phase was transferred to a new well and then 100 μL of the lower phase was transferred to the same well. A correction factor of 1.33 was applied. The chloroform was removed by speedvac and the N-(2-mercaptoethyl)-hexanamide was dissolved in 50 μL assay buffer. 10 μL of the reaction mixtures were mixed with 10 μL of 120 μM ThioGlo in the assay buffer and fluorescence was detected at the microplate reader (ClarioStar, BMG labtech) using the following settings: 400-20 nm; emission: 465-20 nm; gain: 700; focal height: 12.5 mm; flashes: 20; orbital averaging: off.

The concentrations of the three solutions were prepared in either a twofold concentration (Solution 2) or as 4× concentrated stocks (Solution 1 and 3) in the assay buffer. Solution 1 contained the acyltransferases at a concentration of 170 nM, Solutions 2 contained the substrates 3-Oxo-3-[[2-[(1-oxohexyl)amino]ethyl]thio]-propanoic acid in concentrations between 20-1000 nM and Solution 3 contained the acceptor ACP in a concentration of 340 μM. Additionally, TCEP was added to the reaction mixtures at 1 mM final concentration.

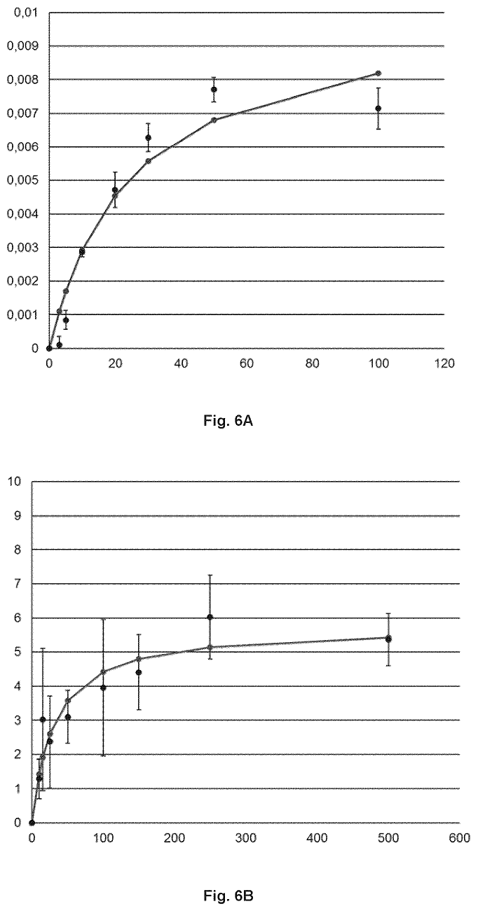

Results of the exemplary kinetic analysis of mutant A35 are shown in FIG. 6 and Table 4.

TABLE 4
Results of kinetic analysis with Mal-CoA (A) and 3-Oxo-3-[[2-[(1-
oxohexyl)amino]ethyl]thio]-propanoic acid (B) corresponding to FIG. 6.

| Substrate | Acceptor | Kₘ [μM] | k_{cat} [s⁻¹*] | k_{cat}/Kₘ [s⁻¹M⁻¹] | Factor |
|---|---|---|---|---|---|
| A | ACP | 25 | 0.01 | 4.07E+02 | 1 |
| B | ACP | 30 | 5.75 | 1.90E+05 | 467 |

Detailed kinetic analysis using non-CoA substrates in comparison to CoA substrates are provided, further in-depth studies for various engineered mutants, including competition assays using both CoA and non-CoA substrates, have been conducted and are partially ongoing.

### REFERENCES

Hertweck C. The biosynthetic logic of polyketide diversity. Angew Chem Int Ed Engl. 2009; 48(26):4688-716. doi: 10.1002/anie.200806121.
Rittner A, Paithankar K S, Huu K V, Grininger M. Characterization of the Polyspecific Transferase of Murine Type I Fatty Acid Synthase (FAS) and Implications for Polyketide Synthase (PKS) Engineering. ACS Chem Biol. 2018 Mar. 16; 13(3):723-732. doi: 10.1021/acschembio.7b00718.
RittnerA, Paithankar K S, HimmlerA, Grininger M. Type I fatty acid synthase trapped in the octanoyl-bound state. Protein Sci. 2020 February; 29(2):589-605. doi: 10.1002/pro.3797.
Smith S, Tsai S C. The type I fatty acid and polyketide synthases: a tale of two megasynthases. Nat Prod Rep. 2007 October; 24(5):1041-72. doi: 10.1039/b603600g.
Yi L, Li H, Sun L, Liu L, Zhang C, Xi Z. A Highly Sensitive Fluorescence Probe for Fast Thiol-Quantification Assay of Glutathione Reductase. Angew. Chem. 2009; 121(22): 4094-4097. https://doi.org/10.1002/ange.200805693.

1. An acyltransferase having altered substrate specificity, wherein the acyltransferase comprises one or more amino acid exchange(s) at reference position(s)
(i) T161 according to SEQ ID NO: 29, or an analogous threonine, serine or asparagine residue at said reference position as defined in Table 1, wherein the amino acid is exchanged to alanine, glycine, valine, leucine, isoleucine, tyrosine, phenylalanine, tryptophan, aspartate or glutamate; and/or
(ii) F184 according to SEQ ID NO: 29, or an analogous phenylalanine residue at said reference position as defined in Table 1, wherein the amino acid is exchanged to alanine, glycine, valine, leucine, isoleucine, methionine, aspartate or glutamate; and/or
(iii) R286 according to SEQ ID NO: 29, or an analogous arginine residue at said reference position as defined in Table 1, wherein the amino acid is exchanged to alanine, glycine, valine, leucine, isoleucine, methionine, aspartate or glutamate; and/or
(iv) K186 according to SEQ ID NO: 29, or an analogous lysine or arginine residue at said reference position as defined in Table 1, wherein the amino acid is exchanged to a negatively charged amino acid or to glycine, alanine, valine, leucine, isoleucine or methionine; and/or
(v) K285 according to SEQ ID NO: 29, or an analogous lysine or arginine residue at said reference position as defined in Table 1, wherein the amino acid is exchanged to a negatively charged amino acid; and/or
(vi) R300 according to SEQ ID NO: 29, or an analogous lysine or arginine residue at said reference position as defined in Table 1, wherein the amino acid is exchanged to a negatively charged amino acid, and/or
(vii) A282 according to SEQ ID NO: 29, or an analogous alanine residue at said reference position as defined in Table 1, wherein the amino acid is exchanged to valine, leucine, isoleucine, phenylalanine or tyrosine; and/or
(viii) G137 according to SEQ ID NO: 29, or an analogous glycine residue at said reference position as defined in Table 1, wherein the amino acid is exchanged to valine, leucine, isoleucine, phenylalanine or tyrosine

2. The acyltransferase of claim 1, wherein
an amino acid exchange at reference position (i) is an exchange to leucine, alanine, tryptophan, or valine, preferably to leucine or alanine, more preferably to leucine; and/or wherein
an amino acid exchange at reference position (ii) is an exchange to leucine, alanine, glycine, valine, or glutamate, preferably to leucine, alanine or glutamate, more preferably to leucine or alanine; and/or wherein
an amino acid exchange at reference position (iii) is an exchange to leucine, alanine, glycine, or valine, preferably to leucine or alanine; and/or wherein
an amino acid exchange at reference position (iv) is an exchange to a negatively charged amino acid, or to alanine or valine, preferably to glutamate or aspartate; and/or wherein
an amino acid exchange at reference position (v) is an exchange to glutamate or aspartate; and/or wherein
an amino acid exchange at reference position (vi) is an exchange to glutamate or aspartate; and/or wherein
an amino acid exchange at reference position (vii) is an exchange to leucine, isoleucine, phenylalanine or tyrosine; and/or wherein
an amino acid exchange at reference position (viii) is an exchange to leucine, isoleucine, phenylalanine or tyrosine, preferably to leucine.

3. The acyltransferase of claim 1, wherein
an amino acid exchange at reference position (i) is an exchange to leucine, isoleucine, tyrosine, phenylalanine or glycine; and/or wherein
an amino acid exchange at reference position (ii) is an exchange to leucine, alanine, isoleucine or methionine; and/or wherein
an amino acid exchange at reference position (iii) is an exchange leucine, alanine, isoleucine or methionine; and/or wherein
an amino acid exchange at reference position (iv) is an exchange to alanine, glycine, valine, leucine, isoleucine or methionine; and/or wherein
an amino acid exchange at reference position (vii) is an exchange to valine; and/or wherein
an amino acid exchange at reference position (viii) is an exchange to valine.

4. The acyltransferase of any of the preceding claims, wherein the acyltransferase is an isolated acyltransferase originating from a fatty acid synthase.

5. The acyltransferase of claim 1, wherein the acyltransferase comprises amino acid exchanges as defined in any one of claims 1 to 3 at reference positions (iv) and (i), or (iv) and (ii), or (iv) and (iii), or (iv) and (v), or (iv) and (vi), or (iv) and (vii), or (iv) and (viii), or (iv) and two or more of reference positions of (i), (ii), (iii), (v), (vi), (vii) and/or (viii), optionally wherein the amino acid exchange at reference position (iv) is an amino acid exchange to glutamate or aspartate.

6. The acyltransferase of claim 1, wherein the acyltransferase comprises amino acid exchanges as defined in any one of claims 1 to 3 at reference positions (i) and (ii), or (i) and (iii), or (i) and (iv), or (i) and (v), or (i) and (vi), or (i) and (vii), or (i) and (viii), or (i) and two or more of reference positions of (ii), (iii), (iv), (v), (vi), (vii) and/or (viii), optionally wherein the amino acid exchange at reference position (i) is as defined in claim 2.

7. The acyltransferase of any of the preceding claims, wherein the acyltransferase comprises an amino acid exchange as defined in claim 1 at reference position (i), optionally wherein the amino acid exchange at reference position (i) is as defined in claim 2.

8. The acyltransferase of claim 7, wherein the acyltransferase further comprises amino acid exchanges as defined in claim 1 at reference positions:
(ii), optionally wherein the amino acid exchange at reference position (ii) is as defined in claim 2; and
(iii), optionally wherein the amino acid exchange at reference position (iii) is as defined in claim 2.

9. The acyltransferase of claim 7 or 8, wherein the acyltransferase further comprises an amino acid exchange as defined in claim 1 at reference position (iv), optionally wherein the amino acid exchange at reference position (iv) is as defined in claim 2.

10. The acyltransferase of any one of claims 7 to 9, wherein the acyltransferase further comprises an amino acid exchange as defined in claim 1 at reference position (vi), optionally wherein the amino acid exchange at reference position (vi) is as defined in claim 2.

11. The acyltransferase of any of the proceeding claims, wherein the acyltransferase comprises amino acid exchanges to glutamate at reference positions (iv) and (vi), or amino acid exchanges to aspartate at reference positions (iv) and (vi), or an amino acid exchange to glutamate at reference position (iv) and an amino acid exchange to aspartate at reference position (vi), or an amino acid exchange to aspartate at reference position (iv) and an amino acid exchange to glutamate at reference position (vi).

12. The acyltransferase of any of the preceding claims, wherein the acyltransferase comprises an amino acid exchange to glutamate or aspartate at reference position (iii) and optionally further comprises an amino acid exchange at reference position: (ix) D160 according to SEQ ID NO: 29, or an analogous aspartate or glutamate residue at said reference position as defined in Table 1, wherein the amino acid is exchanged to arginine, histidine, lysine, glutamine, or asparagine.

13. The acyltransferase of any of the preceding claims, wherein the acyltransferase originates from a fatty acid synthase of an organism selected from vertebrata, including aves and mammalia, including artiodactyla, perissodactyla, carnivora, primates and rodentia, optionally wherein the acyltransferase originates from a fatty acid synthase comprising or consisting of a wild type amino acid sequence selected from any one of SEQ ID NO: 1 to 28, or a sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity thereto.

14. The acyltransferase of any of the preceding claims, wherein the acyltransferase comprises or consists of an amino acid sequence of any of SEQ ID NO: 57 to 82 or 111 to 133, or a sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity thereto.

15. A fusion protein comprising at least one acyltransferase of any one of the preceding claims, preferably wherein the fusion protein further comprises at least one linker domain and optionally at least one ketoacyl synthase domain.

16. A nucleic acid encoding the acyltransferase of any one of claims 1 to 9, or the fusion protein of claim 15.

17. An expression construct or vector comprising at least one nucleic acid of claim 16.

18. A cell comprising at least one acyltransferase of any one of claims 1 to 14; and/or at least one fusion protein of claim 15; and/or at least one nucleic acid of claim 16; and/or at least one expression construct or vector of claim 17.

19. A kit comprising at least one acyltransferase of any one of claims 1 to 14; and/or at least one fusion protein of claim 15; and/or at least one nucleic acid of claim 16; and/or at least one expression construct or vector of claim 17; and/or at least one cell of claim 18; optionally further comprising at least one container; and/or a set of reagents including buffer, medium and/or substrate(s); and/or means of transfection.

20. A use of at least one acyltransferase as defined in any one of claims 1 to 14; and/or at least one fusion protein as defined in claim 15; and/or at least one nucleic acid as defined in claim 16; and/or at least one expression construct or vector as defined in claim 17; and/or at least one cell as defined in claim 18; and/or at least one kit as defined in claim 19; for polyketide synthesis, fatty acid synthesis, and/or non-ribosomal peptide synthesis, optionally wherein it is a use for reacting at least two different substrates.


SEQUENCE LISTING (133 sequences)

SEQ ID NO: 1 - protein, 2504 aa, Mus musculus
MEEVVIAGMSGKLPESENLQEFWANLIGGVDMVTDDDRRWKAGLYGLPKRSGKLKDLSKFDASFFGVHPKQAHTMDPQLRLLLEVSYEAIVDGGINPASLRGTNTGVWVGVSGSEASEALSRDPETLLGYSMVGCQRAMMANRLSFFFDFKGPSIALDTACSSSLLALQNAYQAIRSGECPAALVGGINLLLKPNTSVQFMKLGMLSPDGTCRSFDDSGSGYCRSEAVVAVLLTKKSLARRVYATILNAGTNTDGSKEQGVTFPSGEVQEQLICSLYQPAGLAPESLEYIEAHGTGTKVGDPQELNGITRSLCAFRQAPLLIGSTKSNMGHPEPASGLAALTKVLLSLEHGVWAPNLHFHNPNPEIPALLDGRLQVVDRPLPVRGGNVGINSFGFGGSNVHVILQPNTRQAPAPTAHAALPHLLHASGRTLEAVQDLLEQGRQHSQDLAFVSMLNDIAATPTAAMPFRGYTVLGVEGRVQEVQQVSTNKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLTGINVNPNALFPPVEFPAPRGTPLISPHIKWDHSQTWDVPVAEDFPNGSSSSSATVYSIDASPESPDHYLVDHCIDGRVIFPGTGYLCLVWKTLARSLGLSLEETPVVFENVSFHQATILPKTGTVALEVRLLEASHAFEVSDTGNLIVSGKVYLWEDPNSKLFDHPEVPTPPESASVSRLTQGEVYKELRLRGYDYGPQFQGICEATLEGEQGKLLWKDNWVTFMDTMLQVSILGSSQQSLQLPTRVTAIYIDPATHRQKVYRLKEDTQVADVTTSRCLGITVSGGIHISRLQTTATSRRQQEQLVPTLEKFVFTPHMEAECLSESTALQKELQLCKGLARALQTKATQQGLKAAMLGQEDPPQHGLPRLLAAACQLQLNGNLQLELGEALAQERLLLPEDPLISGLLNSQALKACVDTALENLSTLKMKVAEVLAGEGHLYSRIPALLNTQPMLQLEYTATDRHPQALKDVQTKLQQHDVAQGQWNPSDPAPSSLGALDLLVCNCALATLGDPALALDNMVAALKEGGFLLVHTVLKGHALGETLACLPSEVQPAPSLLSQEEWESLFSRKALHLVGLKRSFYGTALFLCRRAIPQEKPIFLSVEDTSFQWVDSLKSTLATSSSQPVWLTAMDCPTSGVVGLVNCLRKEPGGHRIRCILLSNLSNTSHAPKLDPGSPELQQVLKHDLVMNVYRDGAWGAFRHFQLEQDKPKEQTAHAFVNVLTRGDLASIRWVSSPLKHTQPSSSGAQLCTVYYASLNFRDIMLATGKLSPDAIPGKWASRDCMLGMEFSGRDRCGRRVMGLVPAEGLATSVLLSSDFLWDVPSSWTLEEAASVPVVYTTAYYSLVVRGRIQRGETVLIHSGSGGVGQAAISIALSLGCRVFTTVGSAEKRAYLQARFPQLDDTSFANSRDTSFEQHVLLHTGGKGVDLVLNSLAEEKLQASVRCLAQHGRFLEIGKFDLSNNHPLGMAIFLKNVTFHGILLDALFEEANDSWREVAALLKAGIRDGVVKPLKCTVFPKAQVEDAFRYMAQGKHIGKVLVQVREEEPEAVLPGAQPTLISAISKTFCPAHKSYIITGGLGGFGLELARWLVLRGAQRLVLTSRSGIRTGYQAKHIREWRRQGIQVLVSTSNVSSLEGARALIAEATKLGPVGGVFNLAMVLRDAMLENQTPELFQDVNKPKYNGTLNLDRATREACPELDYFVAFSSVSCGRGNAGQTNYGFANSTMERICEQRRHDGLPGLAVQWGAIGDVGIVLEAMGTNDTVIGGTLPQRISSCMEVLDLFLNQPHAVLSSFVLAEKKAVAHGDGDTQRDLVKAVAHILGIRDLAGINLDSTLADLGLDSLMGVEVRQILEREHDLVLPMREVRQLTLRKLQEMSSKTDSATDTTAPKSRSDTSLKQNQLNLSTLLVNPEGPTLTQLNSVQSSERPLFLVHPIEGSTTVFHSLAAKLSVPTYGLQCTQAAPLDSIPNLAAYYIDCIKQVQPEGPYRIAGYSFGACVAFEMCSQLQAQQGPAPTHNNLFLFDGSHTYVLAYTQSYRAKMTPGCEAEAEAEALCFFIKQFLDVEHSKVLEALLPLKSLEDRVAASVDLITKSHHSLDRRELSFAAVSFYHKLRAADQYKPKAKYHGNVTLLRAKTGGTYGEDLGADYNLSQVCDGKVSVHIIEGDHRTLLEGSGLESIINIIHSSLAEPRVSVREG

SEQ ID NO: 2 - protein, 2505 aa, Rattus norvegicus
MEEVVIAGMSGKLPESENLQEFWANLIGGVDMVTDDDRRWKAGLYGLPKRSGKLKDLSKFDASFFGVHPKQAHTMDPQLRLLLEVSYEAIVDGGINPASLRGTNTGVWVGVSGSEASEALSRDPETLLGYSMVGCQRAMMANRLSFFFDFKGPSIALDTACSSSLLALQNAYQAIRSGECPAAIVGGINLLLKPNTSVQFMKLGMLSPDGTCRSFDDSGNGYCRAEAVVAVLLTKKSLARRVYATILNAGTNTDGCKEQGVTFPSGEAQEQLIRSLYQPGGVAPESLEYIEAHGTGTKVGDPQELNGITRSLCAFRQSPLLIGSTKSNMGHPEPASGLAALTKVLLSLENGVWAPNLHFHNPNPEIPALLDGRLQVVDRPLPVRGGIVGINSFGFGGANVHVILQPNTQQAPAPAPHAALPHLLHASGRTMEAVQGLLEQGRQHSQDLAFVSMLNDIAATPTAAMPFRGYTVLGVEGHVQEVQQVPASQRPLWFICSGMGTQWRGMGLSLMRLDSFRESILRSDEALKPLGVKVSDLLLSTDEHTFDDIVHSFVSLTAIQIALIDLLTSMGLKPDGIIGHSLGEVACGYADGCLSQREAVLAAYWRGQCIKDANLPAGSMAAVGLSWEECKQRCPPGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHVPEHAVVLEIAPHALLQAVLKRGVKPSCTIIPLMKRDHKDNLEFFLTNLGKVHLTGIDINPNALFPPVEFPVPRGTPLISPHIKWDHSQTWDIPVAEDFPNGSSSSSATVYNIDASSESSDHYLVDHCIDGRVLFPGTGYLYLVWKTLARSLSLSLEETPVVFENVTFHQATILPRTGTVPLEVRLLEASHAFEVSDSGNLIVSGKVYQWEDPDSKLFDHPEVPIPAESESVSRLTQGEVYKELRLRGYDYGPHFQGVYEATLEGEQGKLLWKDNWVTFMDTMLQISILGFSKQSLQLPTRVTAIYIDPATHLQKVYMLEGDTQVADVTTSRCLGVTVSGGVYISRLQTTATSRRQQEQLVPTLEKFVFTPHVEPECLSESAILQKELQLCKGLAKALQTKATQQGLKMTVPGLEDLPQHGLPRLLAAACQLQLNGNLQLELGEVLARERLLLPEDPLISGLLNSQALKACIDTALENLSTLKMKVVEVLAGEGHLYSHISALLNTQPMLQLEYTATDRHPQALKDVQTKLQQHDVAQGQWDPSGPAPTNLGALDLVVCNCALATLGDPALALDNMVAALKDGGFLLMHTVLKGHALGETLACLPSEVQPGPSFLSQEEWESLFSRKALHLVGLKKSFYGTALFLCRRLSPQDKPIFLPVEDTSFQWVDSLKSILATSSSQPVWLTAMNCPTSGVVGLVNCLRKEPGGHRIRCILLSNLSSTSHVPKLDPGSSELQKVLESDLVMNVYRDGAWGAFRHFQLEQDKPEEQTAHAFVNVLTRGDLASIRWVSSPLKHMQPPSSSGAQLCTVYYASLNFRDIMLATGKLSPDAIPGKWASRDCMLGMEFSGRDKCGRRVMGLVPAEGLATSVLLSPDFLWDVPSSWTLEEAASVPVVYTTAYYSLVVRGRIQHGETVLIHSGSGGVGQAAISIALSLGCRVFTTVGSAEKRAYLQARFPQLDDTSFANSRDTSFEQHVLLHTGGKGVDLVLNSLAEEKLQASVRCLAQHGRFLEIGKFDLSNNHPLGMAIFLKNVTFHGILLDALFEGANDSWREVAELLKAGIRDGVVKPLKCTVFPKAQVEDAFRYMAQGKHIGKVLVQVREEEPEAMLPGAQPTLISAISKTFCPEHKSYIITGGLGGFGLELARWLVLRGAQRLVLTSRSGIRTGYQAKHVREWRRQGIHVLVSTSNVSSLEGARALIAEATKLGPVGGVFNLAMVLRDAMLENQTPELFQDVNKPKYNGTLNLDRATREACPELDYFVAFSSVSCGRGNAGQSNYGFANSTMERICEQRRHDGLPGLAVQWGAIGDVGIILEAMGTNDTVVGGTLPQRISSCMEVLDLFLNQPHAVLSSFVLAEKKAVAHGDGEAQRDLVKAVAHILGIRDLAGINLDSSLADLGLDSLMGVEVRQILEREHDLVLPIREVRQLTLRKLQEMSSKAGSDTELAAPKSKNDTSLKQAQLNLSILLVNPEGPTLTRLNSVQSSERPLFLVHPIEGSITVFHSLAAKLSVPTYGLQCTQAAPLDSIPNLAAYYIDCIKQVQPEGPYRVAGYSFGACVAFEMCSQLQAQQGPAPAHNNLFLFDGSHTYVLAYTQSYRAKLTPGCEAEAEAEAICFFIKQFVDAEHSKVLEALLPLKSLEDRVAAAVDLITRSHQSLDRRDLSFAAVSFYYKLRAADQYKPKAKYHGNVILLRAKTGGTYGEDLGADYNLSQVCDGKVSVHIIEGDHRTLLEGRGLESIINIIHSSLAEPRVSVREG

SEQ ID NO: 3 - protein, 2503 aa, Cricetulus griseus
MEEVVIAGMSGKLPESENLQEFWANLIGGVDMVTDDDRRWKAGLYGLPKRSGKLKDLSKFDASFFGVHPKQAHTMDPQLRLLLEVSYEAIVDGGINPASLRGTNTGVWVGVSGSEASEALSRDPETLLGYSMVGCQRAMMANRLSFFFDFKGPSIALDSACSSSLLALQSAYQAIRNGECPAALVGGINVLLKPNTSVQFMKLGMLSPEGTCKSFDDSGNGYCRSEAVVAVLLTKKSLARRVYATILNAGTNTDGCKEQGVTFPSGEAQEQLIQSLYRPAGLAPESLEYIEAHGTGTKVGDPQELNGIVRSLCASRQGPLLIGSTKSNMGHPEPASGLAALTKVLLSLENGVWAPNLHFHNPNPEIPALLDGRLQVVDRPLPVRGGNVGINSFGFGGANVHVILQPNTQQAPAPAPHAALPHLLHASGRTAEAVQGLLEQGRKHSQDMAFVSMLNDIAATPTAAMPFRGYTVLGVEGDVQEVQQVPASKRPLWFICSGMGTQWRGMGLSLMRLDSFRESILRSDEAVKPLGIKVSDLLLSTDESTFDDIVHAFVGLTAIQIALIDLLTSVGLKPDGIIGHSLGEVACGYADGCLSQREAVLAAYWRGQCIKDAHLPPGSMAAVGLSWEECKQRCPSGVVPACHNSEDTVTISGPQAAVSEFVEQLKQEGVFAKEVRTGGLAFHSYFMEGIAPMLLQALKKVIREPRPRSARWLSTSIPEAQWQSSLARTSSAEYNVNNLVSPVLFQEALWHVPEHAVLLEIAPHALLQAVLKRGVKSSCTIIPLMKRDHKDNLEFFLTNLGKVHLTGIDVNPNALFPPVDFPAPRGTPLISPHIKWDHSQTWDVPAAEDFPNGSCSSSATVYSIDASPESPDHYLVDHCIDGRVLFPATGYLYLVWKTLARNLSLSLEETPVVFEDVTIHQATILPKTGTVPLEVRLLEASRAFEVSENGNLIVSGKVYQWEDPDSKLFDHPHVLNPPESTPVSRLTQTEVYKELRLRGYDYGPHFQGIVEASLEGEQGKLLWKGNWVTFLDTMLQVSILGVSQQSLRLPVRVTAIHIDPATHLQKVYTLEGENQVADVTTSLCLDSTVSGGVRISRLHTTAAPRRQQEQLVPTLEKFIFTPHVEDGCLSENTTLQRELQLCKDLAQALQTKATQQGLKMTVPGLEGPPQHGLSRLLAAACQLQLNGNLQLELGEALAQERVLLPEDPLISGLLNSQALKACVDTALENLPSLKMKVVEVLSGEGRLYSRIPSLLNTQPMLQLEYTATDLHPEALKDVQAKLQQHDIAQGQWNPEDPAPSNLGALDLVVCSCAVATLGNPALALSNMVAALKEGGFLLMHTVLRGHSLGETLACLPSAVQPGPSLSQEEWESLFSKKGLHLVGLKKSFYGTALFLCRRATLQDNPLFLPVEDTSYQWVDSLKRILAESSSQPVWLTATDCPTSGVVGLVNCLRKEPGGHRIRCILLSNLSTSSHTPKMGPGSSELQKVLKNDLVMNVYRDGAWGAFRHFLLEQDKPEEQTAHAFVNVLTRGDLASIRWVCSPLKHAQPTSPGTQLCTVYYASLNFRDIMLATGKLPPDAIPGKWASRDCMLGMEFSGRDKCGRRVMGLVPAEGLATSVLLSPDFLWEVPSSWTLEEAASVPVVYTTAYYSLVVRGRVQRGETVLIHSGSGGVGQAAISIALSLGCRVFTTVGSAEKRAYLQARFPQLDDTSFANSRDTSFEQHVLLHTGGKGVNLVLNSLAEEKLQASVRCLAQHGRFLEIGKFDLSNNHPLGMAIFLKNVTFHGILLDALFEEANDSWREVAALLQAGIRDGVVKPLKCTVFPKDQVEDAFRFMAQGKHIGKVLVQVREEEPEAVLPGAQPTLIPAISKTFCPAHKSYIITGGLGGFGLELARWLVLRGAQRLVLTSRSGIRTGYQAKQVQEWRRQGIHVLVSTSNASSLEGARALITEATKLGPVGGVFNLAMVLRDAMLENQTPELFQDVNKPKYNGTLNLDRTTREACPELDYFVAFSSVSCGRGNAGQTNYGFANSAMERICEQRRHDGLPGLAVQWGAIGDVGIVLEAMGTNDTVIGGTLPQRIASCMEVLDLFLNQPYAVLSSFVLAEKKAVAQGDGEAQRDLVKAVAHILGIRDLAGVNLDSSLADLGLDSLMGVEVRQILEHEHDLVLPMREVRQLTLRKLEELSSKAGSASETTAPKSKNDTALKQTQLNLSTLLVNPEGPTLTRLNSVQSSERPLFLVHPIEGSTTVFHSLAAKLSVPTYGLQCTQAAPLDSIPDLAAYYIDCIKQVQPEGPYRVAGYSFGACVAFEMCSQLQAQQGPTPTHNSLFLFDGSHTYVLAYTQSYRARMTPGCEAEAEAEAICYFIKQFTDAEHNKVLEALLSLKSLEDRVTAAVDLITKSHHSLDRRELSFAALSFYQKLRAADQYKPKAKYHGNVTLLRAKTGGTYGEDLGADYNLSQVCDGKVSVHIIEGDHRTLLEGSGLESIINIIHSSLAEPRVSVREG

SEQ ID NO: 4 - protein, 2511 aa, Homo sapiens
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDGGINPDSLRGTHTGVWVGVSGSETSEALSRDPETLVGYSMVGCQRAMMANRLSFFFDFRGPSIALDTACSSSLMALQNAYQAIHSGQCPAAIVGGINVLLKPNTSVQFLRLGMLSPEGTCKAFDTAGNGYCRSEGVVAVLLTKKSLARRVYATILNAGTNTDGFKEQGVTFPSGDIQEQLIRSLYQSAGVAPESFEYIEAHGTGTKVGDPQELNGITRALCATRQEPLLIGSTKSNMGHPEPASGLAALAKVLLSLEHGLWAPNLHFHSPNPEIPALLDGRLQVVDQPLPVRGGNVGINSFGFGGSNVHIILRPNTQPPPAPAPHATLPRLLRASGRTPEAVQKLLEQGLRHSQDLAFLSMLNDIAAVPATAMPFRGYAVLGGERGGPEVQQVPAGERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLIKWDHSLAWDVPAAEDFPNGSGSPSAAIYNIDTSSESPDHYLVDHTLDGRVLFPATGYLSIVWKTLARALGLGVEQLPVVFEDVVLHQATILPKTGTVSLEVRLLEASRAFEVSENGNLVVSGKVYQWDDPDPRLFDHPESPTPNPTEPLFLAQAEVYKELRLRGYDYGPHFQGILEASLEGDSGRLLWKDNWVSFMDTMLQMSILGSAKHGLYLPTRVTAIHIDPATHRQKLYTLQDKAQVADVVVSRWLRVTVAGGVHISGLHTESAPRRQQEQQVPILEKFCFTPHTEEGCLSERAALQEELQLCKGLVQALQTKVTQQGLKMVVPGLDGAQIPRDPSQQELPRLLSAACRLQLNGNLQLELAQVLAQERPKLPEDPLLSGLLDSPALKACLDTAVENMPSLKMKVVEVLAGHGHLYSRIPGLLSPHPLLQLSYTATDRHPQALEAAQAELQQHDVAQGQWDPADPAPSALGSADLLVCNCAVAALGDPASALSNMVAALREGGFLLLHTLLRGHPLGDIVAFLTSTEPQYGQGILSQDAWESLFSRVSLRLVGLKKSFYGSTLFLCRRPTPQDSPIFLPVDDTSFRWVESLKGILADEDSSRPVWLKAINCATSGVVGLVNCLRREPGGNRLRCVLLSNLSSTSHVPEVDPGSAELQKVLQGDLVMNVYRDGAWGAFRHFLLEEDKPEEPTAHAFVSTLTRGDLSSIRWVCSSLRHAQPTCPGAQLCTVYYASLNFRDIMLATGKLSPDAIPGKWTSQDSLLGMEFSGRDASGKRVMGLVPAKGLATSVLLSPDFLWDVPSNWTLEEAASVPVVYSTAYYALVVRGRVRPGETLLIHSGSGGVGQAAIAIALSLGCRVFTTVGSAEKRAYLQARFPQLDSTSFANSRDTSFEQHVLWHTGGKGVDLVLNSLAEEKLQASVRCLATHGRFLEIGKFDLSQNHPLGMAIFLKNVTFHGVLLDAFFNESSADWREVWALVQAGIRDGVVRPLKCTVFHGAQVEDAFRYMAQGKHIGKVVVQVLAEEPEAVLKGAKPKLMSAISKTFCPAHKSYIIAGGLGGFGLELAQWLIQRGVQKLVLTSRSGIRTGYQAKQVRRWRRQGVQVQVSTSNISSLEGARGLIAEAAQLGPVGGVFNLAVVLRDGLLENQTPEFFQDVCKPKYSGTLNLDRVTREACPELDYFVVFSSVSCGRGNAGQSNYGFANSAMERICEKRRHEGLPGLAVQWGAIGDVGILVETMSTNDTIVSGTLPQRMASCLEVLDLFLNQPHMVLSSFVLAEKAAAYRDRDSQRDLVEAVAHILGIRDLAAVNLDSSLADLGLDSLMSVEVRQTLERELNLVLSVREVRQLTLRKLQELSSKADEASELACPTPKEDGLAQQQTQLNLRSLLVNPEGPTLMRLNSVQSSERPLFLVHPIEGSTTVFHSLASRLSIPTYGLQCTRAAPLDSIHSLAAYYIDCIRQVQPEGPYRVAGYSYGACVAFEMCSQLQAQQSPAPTHNSLFLFDGSPTYVLAYTQSYRAKLTPGCEAEAETEAICFFVQQFTDMEHNRVLEALLPLKGLEERVAAAVDLIIKSHQGLDRQELSFAARSFYYKLRAAEQYTPKAKYHGNVMLLRAKTGGAYGEDLGADYNLSQVCDGKVSVHVIEGDHRTLLEGSGLESIISIIHSSLAEPRVSVREG

SEQ ID NO: 5 - protein, 2510 aa, Macaca mulatta
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDAGINPASLRGTHTGVWVGVSGSEASEALSRDPETLVGYSMVGCQRAMMANRLSFFFDFKGPSIALDTACSSSMVALQSAYQAISSGQCPAAIVGGINVLLKPNTSVQFLRLGMLSPEGTCKAFDAEGNGYCRSEGVVAVLLTKKSLARRVYATILNAGTNTDGCKEQGVTFPSGEAQEQLIRSLYQSAGVAPESFEYIEAHGTGTKVGDPQELNGIARALCATRQEPLLVGSTKSNMGHPEPVSGLAALAKVLLSLEHGLWAPNLHFHSPHPEIPALLDGRLQVVAQPLPVRGGNVGINSFGFGGSNVHVILRPHTQPPPAPAPHASLPRLLRASGRTPEAVQELLEQGLQHSQELAFLSMLNDIAAIPTTAMPFRGYAVLGGEHSGPEVQQVPAGERPLWFICSGMGTQWRGMGLSLMRLDRFRDSILRSDEAVKPFGLKVSQLLLSMDESTFDDIVHAFVSLTAIQIGLIDLLSCMGLRPDGIIGHSLGEVACGYADGCLSQEEAVLAAYWRGQCIKEAHLPPGAMAAVGLSWEECKQRCPPGVVPACHNSKDTVTISGPQASVFEFMEQLRKEGVFAKEVRTGGMAFHSYFMEAIAPPLLQALKKVIREPKPRSARWLSTSIPEAQWHSSLARTSSAEYNVNNLVSPVLFQEALCHVPEHAVVLEVAPHALLQAVLKRGLKPGCTIIPLMKKDHRDNLEFFLTGIGRLHLSGIDANPNALFPPVEFPAPRGTPLISPLIKWDHSLAWDVPAAEDFPNGSGSPSAAVYNIDTSSESPDHYLVDHTIDGRVIFPATGYLCIVWKTLARALGLAVEQLPVVFEDVVLHQATILPKTGTVSLEVRLLEASRAFEVSQNGNLIVSGKVYQWEDPDPRLFNHPESPTPNPEEPLFLTQAEVYKELRLRGYDYGPHFHGILEASLEGESGRLLWKDNWVSFLDAMLQISILGLAKRSLPLPTRITAIHIDPATHRQKLYTLQDDAQVADVVVSRWLSITVAGGVHMSRVHTESAPRRQQDQQVPILEKFCFTPHVEEGCLSERAALQKELQLCKGLAQALQTKVTQQGLRMVVPGLDGAQVPQDPLQQGLPQLLSAACRLQLNGNLQLELAQVLAQKRPELSEDPLLSGLLDSPALRACLDTAVENMPSRKMKVVEVLAGHGHLYSRIPGLLGAQPLLQLSYTATDRHPQALEAAEAQLQQHDIAQGQWDPADPAPSTLGSTDLLVCNCAVAALGDPASALSNMVATLREGGFLLLHTLLRGHPLGETVAFLTSAEPQYGQGILSQDAWESLFSSVSLRLVGLKKSFYGSALFLCRRPAPQDSPIFLPVDDTSFRWVESLKGILADEDSSRPVWLKAVNCATSGVVGLVNCLRQEPGGHRLRCVLLSNLSSTSRVPEVDPGSAELQKVLQGDLVMNVYRDGAWGAFRHFLLEEDKPEEPTAHAFVTTLTRGDLSSIRWVCSSLRHAQPSRPGAQLCTVYYASLNFRDIMLATGKLSPEAIPGKWASQDNLLGMEFSGRDPSGKRVMGLVPAEGLATSVLLSPDFLWDVPSNWTLEEAASVPVVYCTAYYSLVVRGRVRPGETVLIHSGSGGVGQAAIAIALSLGCRVFTTVGSSEKRAYLQARFPQLDSTSFANSRDTSFEQHVLRHTGGKGVDLVLNSLAEEKLQASVRCLAQHGRFLEIGKFDLSQNHPLGMAIFLKNVTFHGILLDALLDGSAEWQEVAALLQAGIRDGVVQPLKCTVFPGAQVEDAFRYMAQGKHIGKVLVQVLAEEPEAVLKGAKPTLMSAISKTFCPAYKSYIIAGGLGGLGLELAQWLIQRGAQKLVLTSRSGVRTGYQAKQVRQWRRQGVQVLVSTSNISSLEGARGLIAEAAQLGPVGGIFNLTMVLRDAMLENQTPEFFQDVCKPKYSGTLNLDRVTREACPELDYFVVFSSVSSGRGNAGQSNYGFANSAMERICEKRRHEGLPGLAVQWGAIGDVGILADTVNIHATVIGGTLPQRVASCLEVLDLFLNQPHMVLSSFVLAEKAAAYRDRDGEQDLVKAVAHILGIRDLAAINLDSSLADLGLDSLMSVEVRQTLERELNLVLSMREVRQLTLRRLQELSLKADAANDLASPTPKEDGPAQQQTQLNLRSLLVNPEGPTLMRLNSVQSSERPLFLVHPIEGSTTVFHSLASRLSIPTYGLQCTRAAPLDSIHSLAAYYIDCIRQVQPEGPYRVAGYSYGACVAFEMCSQLQAQQNPAPTHNSLFLFDGSHTYVLAYTQSYRAKLTPGCEAEAETEAICFFVQQFTDMEHNRVLEALLPLKGLEERVAAAVDLIIKSHQGLDRQELSFAALSFYYKLRAAEQYTPKAKYHGNVMLLRAKTGGAYGEDLGADYNLSQVCDGKVSVHVIEGDHRTLLEGSGLESIVSIIHSSLAEPRVSVREG

SEQ ID NO: 6 - protein, 2513 aa, Bos taurus
MEEVVITGMSGKLPESENLEEFWANLIGGVDMVTDDDRRWKAGLYGLPRRSGKLKDLSRFDASFFGVHPKQAHNMDPQLRLLLEVTYEAIVDAGINPASIRGTNTGVWVGVSGSEASEALSRDPETLVGYSMVGCQRAMLANRLSFFFDFKGPSITLDTACSSSLLALQRAYQAIQRGECAMAIVGGVNIRLKPNTSVQFMKLGMLSPEGTCKFFDASGNGYCRAKAVMAILLTKKSLARRVYATILNAGTNTDGCKEKGVTFPSGEAQEQLISSLYKPAGLDPETLEYVEAHGTGTKVGDPQELNGIVQALCGTRQSPLRIGSTKSNMGHPEPASGLAALAKVLLSLEHGLWAPNLHFHNPNPKIPALQDGRLQVVDRPLPVLGGNVGINSFGFGGSNVHVILQPNSQPLPPPAPHAALPRLLRASGRTLEGVQGLLELGLQHSQNLAFVSMLNDIATPSPAAMPFRGYAVLGSQGGSQKVQQVLAGKRPLWFICSGMGTQWRGMGLSLMRLSRFRDSILRSDEAVKPLGLQVSQLLLSTDEAIFDDMVISFVSLTAIQIALIDLLTSMGLQPDGIIGHSLGEVACGYADGCISQEEAILSAYWRGQCIKEANIPPGAMAAVGLTWEECKQRCPPGIVPACHNCIDTVTISGPQASMLEFVQQLKQEGVFAKEVRTGGMAFHSYFMDAIAPMLLQQLKKVIREPQPRSPRWLSTSIPETQWQESLARTFSAEYNVNNLVSPVLFQEALWRVPEDAVVLEIAPHALLQAVLKRGLKSSCTIIPLMKKDHRDNLEFFLSNVGQLYLTGIDVNPNGLFPPVEFPAPRGTPLISPHIKWDHSQTWDVPTAEDFPSGSSSSSATIYKIDINPESPDHYLVDHCIDGRIIFPGTGYLCLVWKTLARALDQNMEHTPVVFEDVTLHQAVILPKTGIVLLKVRLLEASCTFEVSENGNLIASGKVYQWEDPNPKLFDNRYGPDPATPVDPTTAIHLSRGDVYKELQLQGFNYGPYFQGILEASSEGNTGQLLWKDNWVTFMDTMLQMSILAPSKRSLRLPTRITAIYIHPATHQQKLYTLQDKTQVADVVINRCLDTTVAGGIYISRIHTSVAPRHQQEQLVPILEKFCFTPHVETGCLAGNLALQEELQLCVGLAQALQTRVAQQGIKMVVPGLDGAQAPQEAPQQGLPRLLATACQLQLNGNLQMEMGQILAQERALLCDDPLLSGLLNSPALKACVTLALENMTSLKMKVVLAGDGQLYSRIPTLLNTQPLLELDYTATDRHPQALEAAQAKLQQLDITQGQWDPSDPAPSNLGGANLVVCNYALASLGDPATAVGNMVAALKEGGFLLLHTLLRGHPLGETVTFLTCPEPQQGQRHLLSQDEWERLFAGASLHLVALKKSFYGSVLFLCRRLAPLDSPIFLPVEDTSFQWVDSLKNILADSSSRAVWLMAVGCTTSGVVGLVNCLRKEPDGHRIRCVLVSNLNSTSPIPETDPKSLELQKVLQSDLVMNVYRDGAWGAFRHFPLEQDKPEEQTEHAFINVLTRGDLSSIRWVCSPLRHSQPTAPGFQLCTIYYASLNFKRNHAGHGQAVPRRHPRNWASRNCLLGMEFSGRDASGKRVMGLVPAEGLATSTLVPQSFLWDVPSNWTLEEAASVPVVYSTAYYALMVRGRMQPGETVLIHSGSGGVGQAAIAIALSLGCRVFPLVGSAEKRAYLQSRFPQLNETSFANSRDTSFEQHVLWHTAGKGADLVLNSLAEEKLQASVRCLAQHGRFLEIGKFDLSKNHPLGMAIFLKNVTFHGILLDSLFEENNTMWQEVSTLLKAGIRKGVVQPLKRTVFPRTQAEDAFRYMAQGKHIGKVVIQVREEEQEAVLHGTKPTQMVALCKTFCPAHKSYIITGGLGGFGLELAHWLVERGAQKLVLTSRSGIRTGYQARQVHEWRRQGVQVLVSTSDVSTLDGTRSLITEAAQLGPVGGIFNLAVVLRDAMLDNQTPEFFQDVNKPKYNGTLNLDRVTREACPELDYFEVFSSVSCGRGNAGQTNYGFANSTMERICEKRRHDGLPGLAVQWGAIADVGLLMELKGTKDKAIGGTLPQRITSCMEVLDLFLNQPHPVLSSFVLAEKATSRGPSGSHQDLVKAVTHILGIRDLATVNLDSSLSDLGLDSLMGVEVRQMLEREHNLLLSMREIRQLTIHKLQEISAQAGTADELTDSTPKFGSPAQSHTQLNLSTLLVNPEGPTLTRLNSVQSSERPLFLVHPIEGSTTVFHSLATKLSIPTYGLQCTGAAPLDSIQSLATYYIECIRQVQPEGNYRIAGYSYGACVAFEMCSQLQAQQNAGPTNNSLFLFDGSHTFVMAYTQSYRAKLNPGCEAEAEAEAMCFFMQQFTEAEHSRVLEALLPLGDLEARVAATVELIVQSHAGLDRHALSFAARSFYHKLRAAEEYTPRATYHGNVTLLRAKMGSAYQEGLGADYNLSQVCDGKVSVHIIEGDHRTLLEGSGLESILSIIHSSLAEPRVSVREG

SEQ ID NO: 7 - protein, 2514 aa, Capra hircus
MEEVVIAGMSGKLPESENLEEFWANLIGGVDMVTDDDRRWKAGLYGLPRRSGKLKDLSRFDASFFGVHPKQAHNMDPQLRLLLEVAYEAIVDAGINPASIRGTNTGVWVGVSGSEAAEALSRDPETLVGYSMVGCQRAMMANRLSFFFDFKGPSIALDTACSSSLLALQRAYQAIQRGECAMAIVGGINILLKPNTSLQFMKLGMLSPEGTRKSFDASGDGYCRAEAVMAVFLTKKSLARRVYATILNAGTNTDGCKKQGVTYPSGEAQEQLISSLYKPAGLDPETLEYIEAHGTGTKVGDPEELNGITQALCGTRQGPLLIGSTKSNMGHPEPASGLAALAKVLLSLEHGLWAPNLHYHNPNPKIPALQDGRLQVVDRPLPVLGGIVGINSFGFGGSNVHVILQPNSQPPPPPGPHAALPRLLRASGRTLEGVQGLLELGLQHSQNLAFVSMLNDIAAPSPAAMPFRGYAVLGSQEGSQEVQQVPTSKRPLWFICSGMGTQWRGMGLSLMRLSSFRDSILRSDEAVKPLGLRVSQLLLSADDIFDDIIVSFVSLTAFQIALIDLLTSMGLRPDGIIGHSLGEVACAYADGCISQEEAILSAYWRGQCIKEANIPPGAMAAVGLTWEECKQRCPPGIVPACHNSIDTVTISGPQASMLEFVKQLKQEGVFAKEVQTGGMAFHSYFMDAIAPTLLQQLKKVIREPQLRSPRWLSTSIPESQWHESLARTFSAEYNVNNLVSPVLFQEALWHVPENAVVLEIAPHALLQAILKRGLQPSCTIIPLMKKDHRDNLEFFLSNVGQLYLTGIDVNPNGLFPPVEFPAPRGTPLISPHIKWDHSQTWDVPAAEDFPNGSSSSSATVYKIDISPESPDHYLVDHCIDGRVLFPATGYLCLVWKTLARALDQNTEHTPVVFEDVMLHQATLLPKTGTVLLEVRLLEASCTFEVSEKGSLIVSGKVYQWEDPDPKLFDNRYGTEPATPVDPTTAIHLRCGDVYKELQLQGFNYGPNFQGILETSSEGNMGQLLWKDNWVTFMDTMLQMSILAPGQRSLRLPTRITAIYIHPATHQQKLYTLQDGTQVADVVMNRCLDTTVAGGVYISRIHTSVAPRHQQEQPVPILEKFCFTPHVESGCLAGNLALQEELQLCVGLAQALQTKVAQQGIKMVVPGLDGTQASQEAPQQGLPQLLATACQLQLNGNLQMGMGQILAQERALLCDDPLLSGLLNSPALKACVDTALENMTSLKMKVVEVLAGDGQLYSRIPALLNTQPLLELDYIATDRHPQALEAAQAKLQQLNITQGQWDPSDPAPSNLGGANLVVCNCALASLGDPATAVGNMVAALKEGGFLLLHTLLRGHPLGETVTFLTCPEPQQGQRHLLSQDEWESLFAGTSLHLVALKKSFYGSVLFLCRRLAPRDSPIFLPVEDTSFQWVDSLKNILADSSSRPVWFMAVGCTTSGVVGLVNCLRKEPDGHRIRCVLVSNLNSTSPIPEIAPKSLELQKVLQSDLVMNVYRDGAWGAFRHFLLEQDKPEEQTEHAFINVLTRGDLSSIRWVCSPLRHSQSTGPGVQLCTIYYASLNFRDIMLATGKLSPDAIPGNWASRNCMLGMEFSGRDASGKRVMGLVPAEGLATSTLVPQSFLWDVPSNWTLEEAASVPVVYSTAYYALIVRGRMQPGETVLIHSGSGGVGQAAIAIALSLGCCVFTTVGSAEKRAYLQSRFPQLNETSFANSRDTSFEQHVLWHTAGKGVDLVLNSLAEEKLQASARCLAQHGRFLEIGKFDLSNNHPLGMAIFLKNVTFHGILLDALFEENSTMWQEVSSLLKAGIREGVVQPLKRTVFPRTQVEDAFRYMAQGKHIGKVVIQVREEEQKAVLHGTKPTQMAALCKTFCPAHKSYVITGGLGGFGLELAHWLVERGAQKLVLTSRSGIRTGYQARQVREWRRQGVQVLVSTRDVSSLDGTQRLITEAAQLGPVGGVFNLAVVLRDAMLDNQTPEFFQDVNKPKYNGTLNLDRVTREACPELDYFVVFSSVSCGRGNAGQTNYGFANSTMERICEKRRHDGLPGLAVQWGAIADVGILVELKGTNDKAIGGTLPQRITSCLEVPDFFLNQPHPVLSSFALAEKAASRGPSGSQQDLVKAVAHILGIRDLATVNPDSSLSDLGLDSLMGVEVRQMLERERSLTLSMREIRQLTIRKLQEISAQAGTADELTNSTHKLSSPAQSQTQLNLSTLLVNPEGPTLTRLNSVQSSERPLFLVHPIEGSTTVFHSLAAKLSIPTYGLQCTRAAPLDSIQSLAAYYIECIRQVQPEGPYRIAGYSYGACVAFEMCSQLQAQQSAGPTNNSLFLFDGSHTFVMAYTQSYRAKMNPGCEAEAEAEAMCFFMRQFMEAEHSRVLEALLPLGDLEARVAATVELIVQRHAGLDRHALSFAARSFYHKLRAAEQYTPRATYHGNVTLLRAKMGGAYGEGLGADYNLSQVCDGKVSVHIIEGDHRTLLEGSGLESILSIIHSSLAEPRVSVREG

SEQ ID NO: 8 - protein, 2489 aa, Equus caballus
MEEVVIAGMSGRLPESENMQEFWANLIGGVDMVTDDDRRWKAGLYGLPRRSGKLKDLSKFDASFFGVHPKQAHTMDPQLRLLLEVTYEAIVDGGINPALLRGTHTGVWVGVSGSEASEALSRDPETLLGYSMVGCQRAMMANRLSFFFDLKGPSITLDTACSSSLLALHNAYQAIRSGECPAAIVGGINVLLKPNTSVQFMKLGMLSPEGACKSFDEAGNGYCRSEAVVAVLLTKKSLARRVYATILNSGTNTDGSKEQGVTFPSGDAQEQLMSSLYKPAGVAPESLEYLEAHGTGTRVGDPQELKGVVRALCATRQDPLLIGSTKSNMGHPEPASGLAALAKVLLSLEHGLWAPNLHFHSPNPEIPALRDGRLQVVDRPLPIRGGNVGINSFGFGGSNVHVILQPNSRLPLVPTPHVALPCLLRASGRTSEAVLGLLEQGRQHSQDLAFVSMLNDIAATPPSAMPFRGYAVLGGEGDAQEVQQVSTGQRPLWFICSGMGAQWRGMGLSLMRLGTFRDSILRSDKAVKPLGLQVSDLLLSADEATFDDTVPGFVSLTAIQIALIDLLTSMGLRPDGIIGHSLGEVACGYADGCVSQEEAILAAYWRGQCIKEANIPPGAMAAVGLSWEECKQRCPPGVVPACHNSKDTVTISGPQAAVSEFVEQLKQEGVFAKEVRTGGLAFHSYFMDSISHTLLQALKKVIREPRPRSVRWLSTSIPEAQWQSSLARTFSAEYNVNNLVSPVLFQEALCHVPEHAVVLEIAPHALLQAVLKRGLKPSCTIVPLMKKDHRDNLEFFLSNVGRLHLMGIDVNPNGLFPPVEFPAPRGTPLISPHIKWDHSQTWYVPSAKDFPSGSSCSSSTVYNIDASPESPDHYLVDHCIDGRTLFPATGYLCLVWRTLAHALGENLEQMPVVFEDVTLYQATILPKTGTMPLEVRLLEASHTFEVSENGTLIVSGKVYQWDNPNPKLFDNRDSLDPADPTDPTATFHLAQGDVYKELRLRGYDYGPHFQGILKANLEGNTGQLLWKDNWVTFLDTMLQMSILGSAQRSLRLPTRIAAIHIDPATHQQKVYTLQGEAQVADLVVDSCLSSTVAGGAIISRLHTSLAPRRQQEQLAPTLEKFCFTPHVEGGCLAGSATLQEELQLCKGLAQALQVKVAQQGLKMVVPGLDGAQVPRESPQQGLPRLLAAACQLQLNGNLQLELGQVLAQESPLLCDDPLLSGLLDSPALKACVDTALENVASLRMKVVEVLAGDGHLYSRIPALLTTQPMLQLDYTATDRHAQALKAAQAKLQQHHVAQGQWDPMDPAPSSLGATDLLVCNCAVATLGDPATALGNMAAVLKEGGFLLLHTLLRGYPLGEMVAFLTCPEPQRGRQGLLSQDEWESLFAGASLRLVALKKSFYGSVLFLCRRPAPQDSPIFLPVEDMSFRWVDSLKNILADLSSRPVWLTAVSCSTSGVIGMANCLRKEPGGHRIRCILVSNLSNTSPVPKMDPSSSELQKVLQGDLVMNVYRDGAWGTFRHFPLEQDQPEEQTEHAFINVLTRGDLSSIRWVCSPLRHAQHPGPGLQLCTVYYASLNFRDIMLATGKLSPDAIPGKWAARDCMLGMEFSGRDASGKRVMGLVPAEGLATSVLLSQDFLWDVPSCWTLEEAASVPVVYTTAYYSLVVRGRVRRGETVLIHSGSGGVGQAAIAIALSLGCRVFTTVGSAEKRAYLQARFPQLNDTSFANSRDTSFEQHVLRHTAGKGVDLVLNSLAEEKLQASVRCLAQHGRFLEIGKFDLSNNHPLGMAVFLKNVTFHGILLDALFDETSSDWQEVVALVRAGIQDGVVQPLKCTVFPKTQVEDAFRYMAQGKHIGKVVVQVREEEPEAVLQEVKPTRMVALSKTFFPAHKSYVVVGGLGGFGLELAQWLVLRGAQKLVLTSRSGIRTGYQAKQVREWRRQGVQVLVSTCNASSLDGARGLIAEAAQLGPVGGVFNLAMVLRDAMLENQTPELFQDVNKPKYNGTLNLDRVTRETCPELDYFVAFSSVSCGRGNAGQTNYGFANSTMERICEKRRHDGLPGLAVQWGAIGDVGIILEAMGANDTVIGGTLPQRIASCLEVLDLFLNQPHPVLSSFVLAEKKAAAHSDSSSQQDLVKAVAHILGIQDLATINLDSSLVDLGLDSLMGVEVRQTLEREHDMVLSMRDIQQLTLRKLQELSSQTCMADELAAPTPKEDGLTRQQAQLNLSTLLVNPEAPTLTRLNSVQSSERPLFLVHPIEGSITVFHSLATKLSIPTYGLQCTKAAPLDSIQSLAAYYIECIRQVQPEGPYRIAGYSYGACVAFEMCSQLQAQQSSAPAHNSLFLFDGSHAYVLAYTQSYRAKLTPGCEAEAETEAMCFFIQQFTDAEHSRVLEALLPLKGLEERVVAAVDLIVQNHAGLDRRVLSFAARSFYYKLRAAEQYMPRATYHGNVTLLRAKTGSAYSEDLGADYNLSQKHDSNCCVDFEISCQLSV

SEQ ID NO: 9 - protein, 2655 aa, Felis catus
MALPAALCEALSQEHPSSRLQRLQGLLGSRYEAAGPWGGWSPQARAPEVPGQCQRPEGPVLPVKSEGWLDQCGLPHTPEASVLASPGRRSWPPGSGSHRSQTRRWCCGVRKPGERLQPGGLAAGSDQRQAPTLLPLHRAAMEEVVIAGMSGKLPESENMEEFWANLIGGVDMVTDDDRRWKAGLYGLPKRTGKLKDLSKFDASFFGVHPKQAHTMDPQLRLLLEVTYEAILDGGINPATLRGTHTGVWVGVSGSEASEALSRDPETLVGYSMVGCQRAMMANRLSFFFDFKGPSIALDTACSSSLLALQNAYQAIRRGECPAAIVGGLNLLLKPNTSVQFMKLGMLSPDGICKAFDESGDGYCRAEAVVAVLLTKKSLARRVYATILNAGTNTDGCKEQGVTFPSGEVQEQLIRSLYEPAGLSPESLEYIEAHGTGTKVGDPQELNGIARALCPSRQEPLLIGSTKSNMGHPEPASGLAALAKVLLSLEHGLWAPNLHFRSPNTQIPALQDGRLQVVQQPLPVRGGNVCINSFGFGGSNVHVILRPNMRPPLAPTRHASLPRLLRASGRTPEAVHCLLEQGRRHSQDLAFVSMLNDIAAISPTAMPFRGYTVLGGKGGSQEVQLVAAGKRPLWFICSGMGAQWCGMGLSLMRLGSFRDSILRSDEAVKPLGLQVSELLLSTDEATFDDIIHAFVSLTAIQIALIDLLNSMGLRPDGIIGHSLGEVACGYADGCLSQEEAILAAYWRGQCIKETNIPPGTMAAVGLSWEECKQRCPPGVVPACHNSEDTVTISGPQAEVAAFVAELKREGVFAKEVRTGGMAFHSYFMDSIAPTLLQALKKVIREPRPRSARWLSTSIPEAQWQGSLARTFSAEYNVNNLVSPVLFQEALWHVPGDAVVLEIAPHALLQAVLKRGLKSSCTIVPLMKKDQRDNLEFFLSNVGKLHLLGFDVNPNGLLPPVEFPVPRGTPLISPHIKWDHSQTWDVPTAKDFPSGSSGSSASTVCNISISPESPDHYLADHCIDGRVLFPATGYLCLVWKTLARTLSQNMEQVPVAFEDVTLHQATVLPKTGTVPLEVRLLQASRTFEVSENGNLIVSGKVYQWEDPDTKLFDSRDGLDATDPMATFHLSQRDVYKELRLRGYDYGPHFQGILEANLEGNAGRLLWKNNWVTFLDAMLQTSILGLAQRSLRLPTRITSVRIDPATHQRRVYSLQGEAQAVNVLVNRCLNSTVAGGVLVSGLHASMAPRRQQEQFAPILEKFCFTPHTEEGLLAGDAALQEGLQLCRGLAQALQTKVAQQGLKMVVSGLDGAQAPQEPPKQGLPRLLAAACQLQLNGGLQLELGQLLAQQRPLLQDDPLLSGLLASPALKACVDTALENMPSLKMKVVEVLAGDGRLYSHIPALLNTQPMLQLEYTATDRHPQALEAVQAKLQQHGIAQGQWDPADHAPGSLGSADLLLCNCAVASLGDPAVAVGNMGAALKEGGFLLLHALLKGHPLGETVAFLTCPEPQRGQQSLLSQDQWESLFAGASLHLVALKKSFYGSVLFLCRRPVLWDSPVFLPVEDPSFQWVNSLKNILADSSQQPVWLTATSCPDSGVVGLVNCLRREPGGHRIRCVLVSNLSSTSSTPSLDPGSSELQKVLRGDLVMNVYRDGAWGAFRHFPLERGLPEEQTEHAFVNVLTRGDLSSIRWVCSPLRHTQLTGPGTHLCAVYYASLNFRDIMLATGKLSPDAIPGKWARHDCLLGMEFSGRDASGKRVMGLVPAEGLATSVLLSQDFLWEVPSSWTLEEAASVPVVYTTAYYSLVVRGRVQPGETVLIHSGSGGVGQAAIAIALSLGCRVFTTVGSAEKRAYLQARFPQLDDTSFANSRDTSFEQHVLRHTAGKGVDLVLNSLAEEKLQASVRCLAQHGRFLEIGKFDLSNNHLLGMAVFLKNVTFHGVLLDSLMDEGGNSWQEVAALLKAGIRDGVVQPLKCTVFPKDRVEDAFRYMAQGKHIGKVVVQVRREEPEAVLRETGPTLMAAVSKTYCPAHKSYVITGGLGGLGLELAQWLVLRGARKLVLTSRSGIRTGYQAKQVREWRRQGIQVLVSTSNASSLDGAQSLIDEATQLGPVGGVFNLAVVLRDAMLENQTPELFEDVVRPKYSGTVNLDRVTRAACPELDYFVAFSSVSCGRGNAGQTNYGFANSTMERVCEKRQHDGLPGLAIQWGAVGDVGIVLESMGSNDVVIGGTLPQRIASCLEVLDVFLNQPHPVLSSFVLAEKKATAHNDSSSRQDPMEAVAHILGIRDLATVNLDSSLADLGLDSLMGVEVRQMLEREHDLVMSMRDIRQLTLRKLQELSSKAGTANELAAPTPKEDTPAGQQAQLNLSTLLVNPEGPTLTRLNSVQSSERPLFLVHPIEGSITVFRSLASKLSIPTYGLQCTRAAPLDSIQSLAAYYIECIRQVQPEGPYRIAGYSYGACVAFEMCSQLQAAQQGPTPAHNSLFLFDGSHTYVLAYTQSYRAKLTPGCEAEAEAEAMCFFVQQFTDMEHNRVLEALLPLKSLEERVAATVDLITRSHAGLDPRELSFAARSFYHKLRAAEQYIPRATYHGNVTLLRTKTGSVYSEGLGADYNLSQVCDGKVSVHVIEGDHRTLLEGSGLETILSIIHSSLAEPRVSVREG

SEQ ID NO: 10 - protein, 2512 aa, Sus scrofa
MEEVVIAGMSGKLPESENLEEFWANLIGGVDMVTADDRRWKAGLYGLPRRMGKLKDLSRFDASFFGVHSKQANTMDPQLRMLLEVTYEAIVDGGINPASLRGTSTGVWVGVSSSDASEALSRDPETLVGYSMIGCQRAMMANRLSFFFDFKGPSITIDTACSSSLLALQSAYQAIRGGECSAAVVGGLNVLLKPNSSLQFMKLGMLSQDGTCRSFDAEGTGYCRAEAVVAVLLTKKSLARRVYATILNAGTNTDGSKEQGVTFPSGDVQEQLIRSLYAPAGPDPESLEYIEAHGTGTKVGDPQELNGIVNALCATRREPLLIGSTKSNMGHPEPASGVAALIKVLLSLEHGVWAPNLHYHTPNPEIPALQDGRLQVVDRPLPIRGGNVGINSFGFGGSNVHVILQPNSRPAPPPAQHAALPRLLQASGRTLEAVQTLLEQGLRHSRDLAFVGMLNEIAAVSPVAMPFRGYAVLGGEAGSQEVQQVPGSKRPVWFICSGMGAQWQGMGLSLMRLDRFRDSILRSDQALKPLGLRVSDLLLSTDEAVLDDIVSSFVSLTSIQIALIDLLTSLGLQPDGIIGHSLGEVACGYADGCLTQEEAVLSSYWRGYCIKEANVLPGAMAAVGLSWEECKQRCPPGIVPACHNSKDTVTISGPQAAMSEFLQQLKREDVFVKEVRTGGIAFHSYFMESIAPTLLRQLRKVILDPKPRSKRWLSTSIPEAQWQGSLARTFSAEYSVNNLVSPVLFQEALQHVPAHAVVVEIAPHALLQAVLKRSLESSCTIIPLMKKDHRDNLEFFLSNVGRLHLAGVSVNPNGLFPPVEFPAPRGTPLISPHXKWDHSQAWDVPSAADFPSGSSCSSVAVYKFDVSPESPDHYLVDHCIDGRVLFPGTGYLWLTWKTLARALSQNLEETPVVFEDVTLHQATILPKTGTVSLEVRLLEASHAFEVSDSNGSLIASGKVYQWESPDPKLFDTRAAVDPADSTAEFRLSQGDVYKDLRLRGYDYGPFFQLVLESDLEGNRGRLQWNDSWVSFLDAMLHMSILAPGQLGLYLPTRFTSIRIDPVTHRQKLYTLQDTTQAADVVVDRNLNTVVAGGALFLGAHSSVAPRRPQEHLKPILEKFCFTPHVESGCLAGNTALQEELQLCRGLAQALQTKVAQQGLKMVVPGLDGAQAPREAPQQSLPRLLAAACQLQLNGNLQLELGQVLAQERPLLCDDPLLSGLLDAPALKACVDTALENMASPKMKVVEVLAGDGQLYSRIPALLNTQPVMDLDYTATDRNPQALEAAQAKLEQLHVTQGQWDPANPAPGSLGKADLLVCNCALATLGDPAVAVGNMAATLKEGGFLLLHTLLAGHPLGEMVGFLTSPEQGGRHLLSQDQWESLFAGASLHLVALKRSFYGSVLFLCRQQTPQDSPVFLSVEDTSFRWVDSLKDILADASSRPVWLMAVGCSTSGVVGMVNCLRKEPGGHRIRCVLVSNLSSTSPAPEMHPSSSELQKVLQGDLVMNVYRDGAWGAFRHFPLEQDRPEKQTEHAFVNVLSRGDLSSIRWVCSPLHYALPASCQDRLCSVYYTSLNFRDVMLATGKLSPDSIPGKWLTRDCMLGMEFSGRDASGRRVMGMVPAEGLATSVLLLQHATWEVPSTWTLEEAASVPIVYTTAYYSLVVRGRMQPGESVLIHSGSGGVGQAAIAIALSRGCRVFTTVGSAEKRAYLQARFPQLDETCFANSRDTSFEQHVLRHTAGKGVDLVLNSLAEEKLQASVRCLAQHGRFLEIGKFDLSNNHALGMAVFLKNVTFHGILLDSLFEEGGATWQEVSELLKAGIQEGVVQPLKCTVFPRTKVEAAFRYMAQGKHIGKVVIQVREEEQGPAPRGLPPIALTGLSKTFCPPHKSYVITGGLGGFGLQLAQWLRLRGAQKLVLTSRSGIRTGYQARQVREWRRQGVQVLVSTSNASSLDGARSLITEATQLGPVGGVFNLAMVLRDAVLENQTPEFFQDVSKPKYSGTANLDRVTREACPELDYFVIFSSVSCGRGNAGQANYGFANSAMERICEKRRHDGLPGLAVQWGAIGDVGVVLETMGTNDTVIGGTLPQRIASCLEVLDLFLSQPHPVLSSFVLAEKKAAAPRDGSSQKDLVKAVAHILGIRDVASINPDSTLVDLGLDSLMGVEVRQILEREHDLVLSMREVRQLSLRKLQELSSKTSTDADPATPTSHEDSPVRQQATLNLSTLLVNPEGPTLTRLNSVQSAERPLFLVHPIEGSITVFHGLAAKLSIPTYGLQCTGAAPLDSIQSLASYYIECIRQVQPEGPYRIAGYSYGACVAFEMCSQLQAQQSATPGNHSLFLFDGSHTFVLAYTQSVRAKMTPGCEAEAEAKAMYFFVQQFTDMEQGKVLEALIPLQGLEARVAATVDLITQSHAGLDRHALSFAARSFYQKLRAAENYWPQATYHGNVTLLRAKTGGAYGEDLGADYNLSQVCDGKVSVHVIEGDHRTLLEGSGLESILSIIHSCLAEPRVSVREG

SEQ ID NO: 11 - protein, 2507 aa, Cuculus canorus
MEEVVIAGMAGKLPESANLQEFWENLMNGVDMVTEDDRRWKPGIYGLPRRNGKLKDLTKFDASFFGVHPKQAETMDPQLRMLLEVSYEAILDAGINPATLRGTDTGVWIGTGGSEAGEALSQDPEELSGYSMTGCQRAMLANRLSYFYDIKGPSVTIDTACSSSLVAMENAFKAIRAGQCHTALVGGCSLLLKPNTSVQFMKLGLLSPNGVCKSFDASGDGYCRSEAIVVLFLTKRSLAKRIYATIVNAGINADGFKEQGVMFPSGEMQQKLASSLLKESGIRPEEVEYVETHGTGTKIGDPQELNGITNVLCRCERAPLLIGSVKSNMGHSEPASGVAALAKVTGGLRRKNGRGQQCVNIPALHDGSMQVVCKPTPVKGGFVSINSFGFGGANANVILKPNMKKQQPVETSNVPRLIQFCGRTQEAVEVLIEESKKHAECIPFISLLSDISGIPASSMPYRGYTILGSESDIREVQQVQSTGRPLWYICSGMGTQWKGMGLSLMKLDLFRQSVLRSDEALKHTGLKVSELLLNEDENTFDDTVHAFVGLAAIQIAQIDILKAAGLEPDGIVGHSVGELACGYADNSLSHEEAILAAYWRGRCVKDAKLPPGGMAAVGLSWEECKQICPPNVVPACHNSEDTVTVSGPLGAVSEFVAKLKKDGVFAKEVRTSGVAFHSHYMASIAPVLLSALKKVIPHPKPRSPRWISTSIPESQWQSDLARYSSAEYHVNNLVNPVLFHEGLSHVPGNALVVEIAPHALLQAILKRSLKPTCIILPLMKKEHKNNLEFFLTQLGKFHMSGVNFNGNNLFPRVEYPVPVETPLISPHIKWDHSQEWYVPQAKDFPSGSKGTSSASVYNIDISPESPDHYLVDHCIDGRVLFPATGYLVLAWRTLARSLGMTIEKMPVTFEDVTIHQATILPKKGAVQLEVRLMPVSHCFEVSGNGNLAVSGKIFVLENDAAKKLDNQPVDCGTQVDVSSKVNLLKEDIYQELHLRGYNYGPNFQGVLEWNSEGTKSKVLWNGNWVTFLDTLLHVVILVESGRSLRLPTRIRTMSIDPVLHQERVCQYKDDVKAIDVVTDRCLASVRAGGVQFSGLHASVAPRRQQERIPPTLEKFCFVPYTEKNCLSSSVHLHDYLKQCKDLIQNLVNKVVVHGIKLVIPGLETADPDAEPLSVQKGLQHILAEMRKLEPNGNLHSELQQIVTREKMHLRDDPLLNGLLDSSELKTCLDVAVENVSSRRMKIIEVLAGSGQLFSRVKSVLTTQPLLQPDYIATDRSLEALSAAETELQDAGVVFSQWDPSSPPAGNLTDADLVVYNCSSVTENTPEIFSNLAAAVKEEGFVLIHTLLRGETVGEFVSFLTSPDLQQKHQFLSEAQWDSLFNSASLNLVAMKKSFFGSVIFLCRRRAPAKTPIFLPVDENDFKWVESLKGILAEQSEQPVWLTATSCGISGILGMVNCLRLEAEGHRIRCLFVSNLKPSSVVPPTSPSCPEMQNIVQKDLVMNVYRDGTWGSFRHLPLQQAQPQELTEYAYVNVLTRGDLSSLRWIASPLRHFQRTNPDVQLCKVYYSSLNFRDIMLATGKLPPDAIPGNWALQQCMLGMEFSGRDMSGRRVMGLLPAKGLATVVDCDKKFLWEVPKNWTLEEAASVPVVYATAYYALVVRGGMKKGESVLIHSGSGGVGQAAIAIALSMGCRVFATVGSTEKREYLQARFPQLDANSFASSRNTSFEQHIMRVTKGKGVNLVLNSLAEEKLQASLRCLAQHGRFLEIGKFDLSNNSQLGMALFLKNVAFHGILLDALFEDGNQDWNIVSELLMKGIKDGVVKPLRSTVFNKEEVEAAFRFMAQGKHIGKVMIKVQEEEKEYPLRRSEPVRISAISRTACPPTKSYILVGGLGGFGLELAQWLVERGAQKLVLTSRSGIRTGYQAKRVEEWKALGVQVLISTRDAATLEGAQQLIEEALRLGPIGGIFNLAVVLRDSMIENQTPELYLEVNKPKYSGTLHLDQVSRKKCPDLDYFVVFSSVSCGRGNAGQSNYGFANSTMERICEQRQHDGLPGLAIQWGAIGDVGVLVKTIGNKDVVVGGTVPQPISSCLEVLDVFLHQPHPVMSSFVLAEKVSVKSDGGSQRDLVEAVGNILGIRDMSTLNPDTSLADLGLDSLMGVEVRQTLERDYDIIMNMREIRLLTINKLRELSSMSGRTEEMKPSQMMKAGFPELPKLNLNNLLVNPEGPTITRLNDVQSTERPLFLIHPIEGSITFFHTLASRLHIPCYGFQCTKAAPLDSIQTLASYYIDCMKQIQPEGPYRIAGYSFGACVAFEMCSQLQAQQNASHTLNNLFLFDGSHSFVAAYTQSYRATLDEGDLAAVETEALSVFVQQFTGMEYNKLLEILLPLEDLAARVNATVDLITQVHTDINREELSFAATSYFYKLKAADQYVPASKYHGNVTLMRVKARSEYEEGLGTDYRLSEVCDGKISVHMVDGDHNTLLEDGVESIVGIIHSSLAEPRVSVREG

SEQ ID NO: 12 - protein, 2511 aa, Calypte anna
MEDVVIAGIAGKLPESENLQEFWENLLNGVDMVTEDDRRWKPGIYGLPRRNGKLKDISKFDASFFGVHPKQAHTMDPQLRLLLEVSYEAILDAGIDPATLRGTDTGVWVGASGSEAAEALSQDPEELLGYSMTGCQRGMFANRISYFYDLTGPSLTIDTACSSSLIALENAYKAIRHGQCSAALVGGVNLLLKPNTSVQFMKLGMLSPEGACKAFDASGNGYCRSEAVVIVLLTKRSMAKRIYATIVNAGTNTDGFKEQGVTFPSGEMQQQLISSLYRESGISPEEVEYVEAHGTGTKAGDPQELNSIVNVFCKCEREPLKIGSTKSNMGHPEPAAGLAALAKVTGGLRRKIGQGRKHNSVGLVCFDIPGLQDGSLEVICKPTPVKGGLVSINSFGFGGANAHVILRPNENRRQPPETCNIPRLVQVCGRTQEAVEVLIQESRKHGGCSPFVSLLSDISAMPVSSMPYRGYTLVGTESDIQEIQQVQASGRPLWYICSGMGTQWKGMGLSLMKLDLFRQSILRSDQALKNTGLKVSDLLLQADDNTFDETVHAFVGLAAIQIAQIDMLKAAGLQPDGILGHSVGELACGYADNCLSHEEAILAAYWRGRCVKEAKLPPGGMAAVGLTWEECKQQCPPNVVPACHNSEDTVTISGPLATVSEFVAKLKKAGVFAKEVRSAGVAFHSHYMASIAPVLLSALKKVIPHPKPRSARWISTSIPESQWQSDLAKYSSAEYHVNNLVSPVLFHEGLKHIPENAVVVEIAPHALLQAILKRTLKPTCTILPLMKKDHKNNLEFFLTQAGKIHLTGINVLGNNLFPPVEYPVPVGTPLISPYIKWDHSQGWDVPKAEDFPSGSKGSASASIYNIDVSPDSPDHYLVGHCIDGRVLYPATGYLVLAWRTLARSLGMVMEQTAVKFEDVTIHQATILPKNGSVQLEVRLMPASHSFEVSGNGNLAVSGKISLLENTALKPADFHTRAGMGLKPGLLKEDIYQELHLRGYNYGPTFQGVLECNSEASAAKVLWNGNWVTFLDTLLHVIILSDTGRSLRLPTRIRSVCVDPVLHQEMVCQYQDNVEVCDAVVDLCLDRLQAGGVQLDGLHASVAPRRQQERMTPTLEKFCFVPYTESDCLSSNAHLHDCLDHCKVLIQKMQAKLAVHGVKLVIPGLDTGSTAAKSSPIQKGLQHILTEICRLELNGNLRSELEQIVTREKMHFHDDPLLSGLLDSSELKSCLDVAVENMTSHRMKIIEALAGSGRLFSRVKSILNTQPLLQVDYIATDRVLETLSASETELQDAGVSSSQWDPSSLPSGNLTNADLVVYNCATNVLGNTTEILSNLAAAVKEGGFVLLHTLLKGETLGEIVSFLSSPDLQQKHKFLHETQWEDLFSKASLNLVAMKKSFFGSVIFLCRRQVPAKTPIFLPVDETHYKWVESLKEILADSSEQPVWLTATSCGNSGILGMVNCLRLESEGHRIRCVFISNLNPSSAVPPTSHSSLEMQKIVQGDLVMNVYRDGKWGSFRHLPLQQAQPQELTEYAYVNVLTRGDLSSLRWIVSPLQHFQANNPDIQLCKVYYASLNFRDIMLATGKLSPDAIPGNWVLQQCMLGMEFSGRDMAGRRVMGLLPAKGLATVVDCEKKFLWEVPKNWTLEEAASVPVVYATAYYALVVRGRMKKGESVLVHSGSGGVGQAAIAIALSMGCRVFTTVGSAEKREYLQARFPQLDANSFASSRDTAFEQHVLRVTNGKGVNLVLNSLAEEKLQASLRCLARHGRFLEIGKFDLSNNSQLGMALFLKNVAFHGILLDAIFEGGNQEWDIVSELLTKGIKDGVVKPLKSTVFKKEEVEAAFRFMAQGKHIGKVMIQIQEEEKEYSVRRSEPVKISAISRTSCPPSKSYIITGGLGGFGLELAQWLVERGAQKLILTSRSGIRTGYQARRVREWKALGIQVLVSTSDIGTLEGAQRLIEEALQLGPVGGIFNLAVVLRDAMIENQTPELFVEVNKPKYSGTLHLDWVTRKKCPDLDYFVVFSSVSCGRGNAGQSNYGFANSTMERICEQRHHDGLPGLAIQWGAIGDVGILLKTMGNKEVVVGGTIPQQISSCLEVLDTFLNQPHPVTSSFVLAEKVSVKAEGGSQRDLVEAVAHILGVRDVSSLNADSSLADLGLDSLMGVEVRQTLERDYDIVMTMREIRLLTINKLRELSSKSGTTEELKPSQVMKTGAGEPPKLDLNNLLVNPEGPTITRINDVQSTERPLFLVHPIEGSTAVFKTLASKLHMPCYGFQCTKVAPLDSIQSLATYYINCMKQIQPEGPYRIAGYSFGACVAFEMCSQLQAQHKGSHALNSLFLFDGSHSFVAAYTQRYRAKLSQGNEAALETEALCAFVQQFTGIEYNKLLEILLPLKDLEARVNAAADLITQVHTHINREALCFAATSFYHKLKAADKYIPESKYHGNVTLVRAKSHNEYEEGLGGDYRLSEVCDGKVSVHVVEGDHRTMLEGNGAESIIGIIHSSLAEPRVSVREG

SEQ ID NO: 13 - protein, 2513 aa, Anas platyrhynchos
MEDVVIAGIAGKLPESENLQEFWENLLNGVDMVTEDDRRWKPGMYGLPKRNGKLKDISKFDASFFGVHPKQAHTMDPQLRLLLEVSYEAILDGGINPATLRGTDTGVWVGASGSEAAEAFSQDPEQLLGYSMTGCQRAMFANRISYFFDFKGPSLTIDTACSSSLMALENAYKAIRNGQCSAAVVGGVNLLLKPHTSVQFMKLGMLSPDGACKAFDASGNGYCRSEAVVVVLLTKKSMAKRIYATIVNAGSNTDGFKEQGVTFPSGDMQRQLVSSLHRECGIKPGDIEYVETHGTGTKVGDPQEVNGIADVFCQCEREPLLIGSTKSNMGHPEPASGLAALAKVVLSLEHGLWAPNLHFSDPNPDIPALLNGSLKVVCEPTPTKGGLVSINSFGFGGANVHVILKPNEKKHQPPETCNLPRLVQVCGRTQEAVETLIQESKKQRGCGPFLRLLTDISAIPVSLMPYRGYTLVGTESDIGEVQQVQVSGRPLWYICSGMGTQWKGMGLSLMKLDVFRQSIMRSDEALKNTGLKVSDLLLQADDNTFDETVNAFVGVAAVQIAQIDMLKAAGLQPDGILGHSVGEVACGYADNSLSHEEAILAAYWRGRCVKEAKLPPGGMAAVGLTWEECKQRCPPNVVPACHNSEDTVTISGPLDSVNEFVAKLKKDGVFAKEVRSAGVAFHSYYMASIAPALLSALRKVIPHPKPRSARWISTSIPESQWQSDLARNSSAEYYVNNLVSPVLFHEGLKHIPENAVVVEIAPHALLQAILRRSLKPSCTILPLMKKDQKNNLEFFLTQAGKIHLTGINVLGNNMFPSVEYPVPVGTPLISPYIKWDHSQDWDVPKAEDFPSGSKGSASASVYNIDVSPDSPDHYLVGHCIDGRILYPATGYLVLAWRSLAGSLGMAMEQTAVMFEDVTIHQATILPKKGSVQLEVRIMPASHCFEVSGNGNLAVSGKINLLENDALKNFRNQLAAFQTHLDTSSKSGLLKEDVYQELHLRGYNYGPTFQGVLECNSEGSAGKVLWNGNWVTFLDTLLHMIVLAETGRSLRLPTRIRSVYIDPVLHPEHVCQYQDNVEALDVVVDHCLNSLKAGGVQINGLHASVAPRRQQERIPPTLEKYCFIPYIESNCLSSSAQLHAYLEHCKGLIKNLQAKVAVHGVKLIIHGLETAGAAAGSSPMQKGLQHILAEICHLELNGNLHSELEQIVTQEKMHFQDDPLLNGLLDSSELKACLDVALENMTSHRMKIVEALAGNGHLFSRVTSILNTQPLLHLDYIATDCTLEALSANETELQDAGISFSQWDPSNPPSGNLSNADLVVYNCSTNVLGNTTKIVSNLAAAVKEGGFVLLHTLLQGETLGEIVSFLTSPDLQQKHSFLSQAQWEGLFSKASLNMIAMKRSFFGSVIFLCRRQSPAKTPIFLPIDETHYKWVESLKEILADQSEQPVWLTATSCGNSGILGMVNCLRLEAEGHRVRCVFVSNLNPSSAVPSISLSSLEMQEIVQRDLVMNVYRDGKWGSFRHLPLQQGQPQELTEYAYVNALTRGDLSSLHWIVSPLQHFYTTNPDVQLCKVYYASLNFRDIMLATGKLSPDAIPGNWASQQCMLGMEFSGRDMAGRRVMGLVSAKGLATVVDCNKRFLWEVPKNWTLEEAASVPVVYATAYYALVVRGGMKKGESILIHSGSGGVGQAAIAIALSMGCRVFTTVGSAEKRQYLQARFPQLDANSFASSRNTTFEQHVLRVTNGKGVNLVLNSLAEEKLQASLRCLAQHGRFLEIGKFDLSNNSQLGMALFLKNVSFHGILLDSIFGESNEEWEVVSKLLTKGIENGVVKPLRSTVFNKDEVEAAFRFMAQGKHIGKVMIKVQEEEKQYPLRRSEPVKLSAISRTSCPPTKSYIITGGLGGFGLELAQWLIERGAQKLVLTSRSGIRTGYQAKCVREWKALGIQVLVSTSDIGTLEGTQLLIEEALRLGPVGGIFNLAVVLRDAMIENQTPELFWEVNKAKYSGTLHLDWVTRKKCPDLDYFVVFSSVSCGRGNAGQSNYGFANSAMERICEQRHHDGLPGLAIQWGAIGDVGILKTVGNTDVVIGGTIPQQISSCLEVLDTFLNQSHPVVSSFVLAEKVSVKSEGSNQRDLVEAVAHIIGVRDVSSLNADSTLADLGLDSLMGVEVRQTLERDYDIIMTMREIRLLTINKLRELSSKSGTAEELKPSQLLKTGPGEPPKLDLNNLLVNPEGPTITRLNDVQSTERPLFLVHPIEGSIAVFYTLASRLHMPCYGLQCTKAAPLDSIQSLASYYIDCMKQIQPEGPYRIAGYSFGACVAFEMCSQLQGQQNASHALNSLFLFDGSHSFVAAYTQSYRAKLAQGNEAALETEALCAFVQQFTGIEYNKLLEILLPLKDLEARVNAAADLITQIHKNINREALSFAAASFYHKLKAADKYIPESKYHGNVTLMRAKTHNEYEEGLGGDYRLSEVCDGKVSVHVIEGDHRTLLEGDGVESIIGIIHSSLAEPRVSVREG

SEQ ID NO: 14 - protein, 2512 aa, Gallus gallus
MEDVVIAGIAGKLPESENLQEFWENLLNGVDMVTEDDRRWKPGIYGLPKRNGKLKDIKKFDASFFGVHPKQAHTMDPQLRLLLEVSYEAILDGGINPTALRGTDTGVWVGASGSEALEALSQDPEELLGYSMTGCQRAMLANRISYFYDFTGPSLTIDTACSSSLMALENAYKAIRHGQCSAALVGGVNILLKPNTSVQFMKLGMLSPDGACKAFDVSGNGYCRSEAVVVVLLTKKSMAKRVYATIVNAGSNTDGFKEQGVTFPSGEMQQQLVGSLYRECGIKPGDVEYVEAHGTGTKVGDPQEVNGIVNVFCQCEREPLLIGSTKSNMGHPEPASGLAALAKVILSLEHGLWAPNLHFNDPNPDIPALHDGSLKVVCKPTPVKGGLVSINSFGFGGSNAHVILRPNEKKCQPQETCNLPRLVQVCGRTQEAVEILIEESRKHGGCSPFLSLLSDISAVPVSSMPYRGYTLVGTESDITEIQQVQASGRPLWYICSGMGTQWKGMGLSLMKLDLFRQSILRSDEALKSTGLKVSDLLLNADENTFDDTVHAFVGLAAIQIAQIDVLKAAGLQPDGILGHSVGELACGYADNSLSHEEAVLAAYWRGRCVKEAKLPPGGMAAVGLTWEECKQRCPPNVVPACHNSEDTVTVSGPLDSVSEFVTKLKKDGVFAKEVRRAGVAFHSYYMASIAPALLSALKKVIPHPKPRSARWISTSIPESQWQSDLARNSSAEYHVNNLVNPVLFHEGLKHIPENAVVVEIAPHALLQAILRRTLKPTCTILPLMKKDHKNNLEFFLTQTGKIHLTGINVLGNNLFPPVEYPVPVGTPLISPYIKWDHSQDWDVPKAEDFPSGSKGSASASVYNIDVSPDSPDHYLVGHCIDGRVLYPATGYLVLAWRTLARSLGMVMEQTAVMFEEVTIHQATILPKKGSTQLEVRIMPASHSFEVSGNGNLAVSGKISLLENDALKNFHNQLADFQSQANVTAKSGLLMEDVYQELHLRGYNYGPTFQGVLECNSEGSAGKILWNGNWVTFLDTLLHLIVLAETGRSLRLPTRIRSVYIDPVLHQEQVYQYQDNVEAFDVVVDRCLDSLKAGGVQINGLHASVAPRRQQERISPTLEKFSFVPYIESDCLSSSTQLHAYLEHCKGLIQKLQAKMALHGVKLVIHGLETKGAAAGSPPAQKGLQHILTEICRLELNGNPHSELEQIVTQEKMHLQDDPLLNGLLDSSELKTCLDVAKENTTSHRMKIVEALAGSGRLFSRVQSILNTQPLLQLDYIATDCTPETLSDNETELHDAGISFSQWDPSSLPSGNLTNADLAVCNCSTSVLGNTAEIISNLAAAVKEGGFVLLHTLLKEETLGEIVSFLTSPDLQQKHSFLSQAQWEELFSKASLNLVAMKRSFFGSVIFLCRRQSPAKAPILLPVDDTHYKWVDSLKEILADSSEQPLWLTATNCGNSGILGMVNCLRLEAEGHRIRCVFVSNLSPSSTVPATSLSSLEMQKIIERDLVMNVYRDGKWGSFRHLPLQQAQPQELTECAYVNVLTRGDLSSLRWIVSPLRHFQTTNPNVQLCKVYYASLNFWDIMLATGKLSPDAIPGNWTLQQCMLGMEFSGRDLAGRRVMGLLPAKGLATVVDCDKRFLWEVPENWTLEEAASVPVVYATAYYALVVRGGMKKGESVLIHSGSGGVGQAAIAIALSMGCRVFATVGSAEKREYLQARFPQLDANSFASSRNTTFQQHILRVTNGKGVSLVLNSLAEEKLQASLRCLAQHGRFLEIGKFDLSNNSQLGMALFLKNVAFHGILLDSIFEEGNQEWEVVSELLTKGIKDGVVKPLRTTVFGKEEVEAAFRFMAQGKHIGKVMIKIQEEEKQYPLRSEPVKLSAISRTSCPPTKSYIITGGLGGFGLELAQWLIERGAQKLVLTSRSGIRTGYQAKCVREWKALGIQVLVSTSDVGTLEGTQLLIEEALKLGPVGGIFNLAVVLKDAMIENQTPELFWEVNKPKYSGTLHLDWVTRKKCPDLDYFVVFSSVSCGRGNAGQSNYGFANSAMERICEQRHHDGLPGLAVQWGAIGDVGILKAMGNREVVIGGTVLQQISSCLEVLDMFLNQPHPVMSSFVLAEKVSVKSEGGSQRDLVEAVAHILGVRDVSSLNAESSLADLGLDSLMGVEVRQTLERDYDIVMTMREIRLLTINKLRELSSKTGTAEELKPSQVLKTGPGEPPKLDLNNLLVNPEGPTITRLNEVQSTERPLFLVHPIEGSIAVFYTLASKLHMPCYGLQCTKAAPLDSIQSLASYYIDCMKQIQPEGPYRIAGYSFGACVAFEMCSQLQAQQNASHALNSLFLFDGSHSFVAAYTQSYRAKLTQGNEAALETEALCAFVQQFTGIEYNKLLEILLPLEDLEARVNAAADLITQIHKNINREALSFAAASFYHKLKAADKYIPESKYHGNVTLMRAKTHNEYEEGLGGDYRLSEVCDGKVSVHIIEGDHRTLLEGDGVESIIGIIHGSLAEPRVSVREG

SEQ ID NO: 15 - protein, 2509 aa, Nestor notabilis
MEDVVIAGIAGKLPESENLQEFWENLLNGVDMVTEDDRRWKPGIYGLPKRNGKLKDISKFDASFFGVHPKQAHTMDPQLRLLLEVSYEAILDGGINPATLRGTDTGVWIGASGSEAAEALSQDPEKLLGYSMTGCQRAMLANRISYFYDFTGPSIAIDTACSSSLIALENAFKAIRHGQCSAALVGGVNLLLKPNTSVQFMKLGMLSSDGACKAFDASGSGYCRSEAVVVVLLTKRSMAKRIYATIVNAGSNTDGFKEQGVTFPSGEMQQRLVSSLYRECGIKLEEVEYVEAHGTGTKVGDPQEINGIVKMFHQCERAPLLIGSTKSNMGHPEPASGLAALAKVMGGLRRRIEGGREHNNIPALQDGTLEVVCKPTPVKGGLVGINSFGFGGANAHVILRPHKNKCQPLETCNTPRLVQICGRTQEAVETLIQESREHGGCSPFLSLLTDISAVPVSSMPYRGYTLVGTESDIKEVQHVQASGRPLWYICSGMGSQWKGMGLSLMKLDMFRQSILRSDEALKNTGLKVSDLLLRADENTFDETINAFVGLAAIQIAQIDLLKAAGLQPDGILGHSVGELACGYADNCLSQEEAILGAYWRGRCVKEAKLPPGAMAAVGLTWEECKQRCPPNVVPACHNSEDTVTVSGPLESVTEFVAKLKKDGVFAKEVRSAGVAYHSHYMASIAPVLLNALKKVIPHPKPRSARWISTSIPESQWQSDLARSCSAEYLVNNLVNPVLFHDGLKHVPENAVVVEIAPHALLQAILRRTLKPTCTILPLMKKDQKNNLEFFLTQTGKIHLTGINVLGNNLFPLVEYPVPVGTPLISPYIKWDHSQDWDVPKAEDFPAGSKGSASASVYSIDLSPDSPDHYLVGHCIDGRVLYPATGYLVLAWRTLARSLGMAMEQMAVTFEEVTIHQATILPKNGSVQLEVRLMPASHYFEVSGNGNLTVSGKIFLLENTALKNFHNQLVDFQTQADTSSKSGLLKEDIYQELHLRGYNYGPTFQGVLECNSEASTGKVLWNGNWVTFLDTLLHMMVLAKSGRSLRLPTRIRSVCIDPVLHQEKVCQYQDNVEALDVVVDPCLGSLKAGGVQIDGLHASVAPRRQQERIPPTLEKFCFVPYTESNCLSSSVHLRAYLEHCKDLIRNVQAKVAVHGVKLVIPGLKTAVAAAGSSPIQKGLQHILAEICRLELNGNLSSELEQIVTQEKMHFQDDPLLNGLLDSSELKTCLGVALENMTSHRMKIVEALAGSGCLFSRIKSILNAQPLLQVDYIATDRILKTLSDSETELQDAGVSISQWDPSCLPSGDLANADLVVYNCLTVLGNTTEIVSNLAAAVKEGGFFLLHTLLKGETFGEIVSFLTSPDLQQKHSFLSEAQWEGLFSKASLNVVAMKKSFFGSVIFLCRRQAPAKTPIFLPVDETHFAWVESLKEVLADPSEQPVWLTATSCGNSGILGMVNCLRLEAEGHRIRCVFVSNLNPSSAVPPTSLSSLEMQKIVQRDLVMNVYRDGKWGSFRHLPLQQAQSQELTEYAYVNVLTRGDLSSLHWIVSPLRHFHTTNPNVQLCKVYYASLNFRDIMLATGKLPPDAIPGNWVLQQCMLGMEFSGRDLAGRRVMGLLPAKGLATVVDCDKRFLWEVPKNWTLEEAASVPVVYATAYYALVVRGGMKKGESILIHSGSGGVGQAAIAIALSMGCRVFTTVGSTEKREYLQARFPQLDANSFASSRNTTFEQHILRLTNGKGVNLVLNSLAEEKLQASLRCLAQHGRFLEIGKFDLSNNSQLGMALFLKNVAFHGILLDAIFEENQAWEVVSELLKKGIKDGVVKPLRSTIFNKDEVEAAFRFMAQGKHIGKVMIKVQEEELESPLRRSDPVKISAISRTSCPPTKSYIITGGLGGFGLELAQWLVERGAQKLILTSRSGIRTGYQADRVRQWKALGVQVLVSTSDIATLEGTQLLIEEALQLGPVGGIFNLAVVLRDGMIENQTPELFCEVNKPKYSGTLHLDWVTRKKCPDLDYFVVFSSVSCGRGNAGQSNYGFANSTMERICEQRHHDGLPGLAIQWGAIGDVGILLKTMGDREIVIGGTLPQQISSCLEVLDIFLNQPHPVMSSFVLAEKVSVKTEGGSQQDLVAAVAHILGVRDVSSLNAESTLADLGLDSLMGVEVRQTLERDYDIVMTMREIRLLTVNKLRELSSKSRTAEGIVPARAQLMKTGPGKSPKLDLNNLLVNPEGPTITRLNDVQSTERPLFLVHPIEGSVAVFYTLSSRLHMPCYGLQCTKAAPLDSIQSLAAYYIDCMKQIQPEGPYRIAGYSFGACVAFEMCSQLQDQQNASHALNSLFLFDGSHSFVAAYTQSYRAKLAQGNEAALETEALCAFVQQFTGIEYNKLLEVLLPLKDLEARVNAAADLITQIHANINREALSFAAASFYHKLKAADKYIPESKYRGNVTLMRAKSHSEYEEGLGGDYRLSEVCDGKVSVHIIEGDHRTLLEGDGVESIIGIIHSSLAEPRVSVREG

SEQ ID NO: 16 - protein, 2516 aa, Podiceps cristatus
MEDVVIAGIAGKLPESENLQEFWENLLNGVDMVTEDDRRWKPGIYGLPKRNGKLKDISKFDASFFGVHPKQANTMDPQLRLLLEVSYEAILDAGINPTTLRGTNTGVWVGASGSEAAEAFSRDPEELLGYSMTGCQRAMLANRISYFYDLKGPSLVIDTACSSSLVALESAYKAIRNGRCSAALVGGANLLLKPNTSVQFMKLGLLSPDGTCKAFDASGNGYCRSEAVVVILLTKRSMAKRIYATIVNAGINTDGFKEQGVTFPSGEMQQQLISSLYKECNIKPEELEYIETHGTGTKVGDPQEINGIADLFCQLERPPLLIGSTKSNMGHPEPASGLAALAKVTGGLRRKIGGEGEHNGAWVFVNIPALQDGSLEVICKPTPVKGGLVGINSFGFGGANAHVILRPNEKKSQPLETCNMPRLVQFCGRTKEAVETLIQASRNHGACSPFVSLLSDISTLPVSSMPYRGYALVGTDDSDIKEVQQVQGSGRPLWYICSGMGTQWKGMGVDLMKLDLFRQSILRSDEALKSTGLKVSDLLLQADENTFDDTVHAFVGLAAIQIAQIDMLKAAGLQPDGILGHSVGELACGYADNSLSREEAILAAYWRGRCVKEAKLPPGGMAAVGLTWEECKQCCPPNVVPACHNSEDTVTISGPLDSVHEFVAKLKKDGVFAKEVRSAGVAFHSYYMASIAPVLLSALKKVIPHPKPRSARWISTSIPESQWQSDLARNSSAEYHVNNLVNPVLFHEGLKHVPENAVVVEIAPHALLQAILRRTLKPTCTILPLMKKDHKNNLEFFLTQTGKIHLTGINVLGNNLLPLVEYPVPVGTPLISPYIKWDHSQDWDVPKAEDFPSGSKGSASASVYNIDVSPDSPDHYLVGHCVDGRVLYPATGYLVLAWRTLARSLGMAMEQMAVMFEEVTIHQATILPKKGSIQLEVRLMPASHCFEVSGNGNLAVSGKISLLENTALKDFHNQIVDFQTRVDRSSKSGLLKEDIYQELHLRGYNYGPTFQGVLECNSEASMGKVLWNRNWVTFLDTLLHMIVLTETGRSLRLPTRIRSVCIDPVLHQEYVCQYQDNVEAFDVVVDRCLDSLRAGGVQINGLHASVAPRRQQERTPPTLEKFCFVPYTESNCLSSSVHLNAYLEHCKGLIRNLQAKVAVHGVKLVIPGLETSVAAAKSSPIQKGLQHILAEICHLELNGNLHSELEQIVTQEKMHLQDDPLLNGLLDSSELNTCLDVALENMTSHRIKIVEALAGSGHLFSRVKSILNTRPLLQVDYIATDCILETFSANETELQDAGVSFSQWDPSSLPSGNLTNADLVVYNYSRSVLGNITEILSNLVAAVKEGGFVLLHTLLKGGTLGDIVSFLTSPDLQQKHSFLSEEQWEGLFSKASLNLVAMKKSFFGSVIFLCRRQAPAKTPIFLPVDETHYKWVESLKEILANPSEQPVWLTATSCGNSGILGMVNCLRLEAEGHRIRCVFVTNLNPSSAVPPTSLSSLEMQKIVQRDLVMNVYRDGKWGSFRHLPLQQAQPQELTEYAYVNVLTRGDLSSLHWIVSPLQHFHATNPNVQLCKVYYASLNFRDIMLATGKLSPDAIPGNWALQQCMLGMEFSGRDLAGRRVMGLLPAKGLATVVDCDKKFLWEVPKNWTLEEAASVPVVYATAYYALVVRGGMKKGESILIHSGSGGVGQAAIAIALSMGCRVFATVGSTEKREYLQARFPQLDANSFASSRNTTFEQHILRVTNGKGVNLVLNSLAEEKLQASLRCLARHGRFLEIGKFDLSNNSQLGMALFLKNVSFHGILLDAVFEDGNEEWEVVSELLKKGIKDGVVKPLRSTVFNKEEVEAAFRFMAQGKHIGKVMIKVQEEEKEYLLRKSEPVKVSAISRTSCPPTKVYIITGGLGGFGLELAQWLVERGAQKLILTSRSGIRTGYQAKCVGDWKALGIQVLVSTSDIATLEGTQLLIEEALQLGPVGGIFNLAVVLRDSMIENQTPESFWEVNKPKYSGTLHLDWVTRKKCPDLDYFVVFSSVSCGKGNAGQSNYGFANSTMERICEQRHHDGLPGLAIQWGAIGDVGILLKTIGNKDVVIGGTVPQQISSCLEVLDIFLNQPHPVMSSFVLAEKVSVKPQEGSQRDLVEAVAHILGVRDVSTLNAESSLADLGLDSLMGVEVRQTLERDYDIVMTMREIRNLTINKLRELSSKSGTAEELKPSQLTKTGPGEPPKLDLNNLLVNPEGPTITRLNDVQSTERPLFLVHPIEGSIAVFSTLASRLHLPCYGLQCTKAAPLDSIQSLAAYYIDCMKQIQPEGPYRIAGYSFGACVAFEMCSQLQAQQNASHALNSLFLFDGSHSFVTAYTRSYRAKLTQGNEAALETEALCAFVQQFIGIEYNKLLEVLLPLKDLDARVNAAADLITRIHTNINHEALSFAAASFYHKLKAADKYIPESMYHGNVTLMRAKSYNEYEEGLGGDYRLSEVCDGKVSVHVIEGDHRTLLEGDGVESIIGIIHSSLAEPRVSVREG

SEQ ID NO: 17 - protein, 2518 aa, Balearica regulorum
  source 1..2518: note = Balearica regulorum gibbericeps
MEDVVIAGIAGKLPESENLQEFWENLLNGVDMVTEDDRRWKPGIYGLPKRNGKLKDISKFDASFFGVHPKQAHTMDPQLRLLLEVSYEAILDGGINPATLRGTDTGVWIGASGSEAGEALSQDPEELLGYSMTGCQRAMFANRISYIYDLTGPSITIDTACSSSLVALENAYKAIRHGRCSAALVGGVNLLLKPNTSVQFMKLGMLSPDGACKAFDASGNGYCRSEAVVVILLTKRSMAKRIYATIVNAGSNTDGFKEQGVTFPSGEMQQQLVSSLYRECGIKPGEVEYVEAHGTGTKAGDPQEVNGIVSVFCQCEREPLLIGSTKSNMGHPEPASGLAALAKVTGGLKKKIGEGTHAHPNVYVFLNVDIPALQDGSLEVVCKPTPVKGGIVSINSFGFGGANAHVILRPNENKRQPLETCNMPRLVQICGRTQEAVEILIQESRKHGGCSPFVSLLSDISAVPVSSMPYRGYTLVGTESDIKEVQQVQASGRPLWYICSGMGTQWKGMGLSLMKLDLFRQSILRSDEALKNTGLKVSDLLLQADENTFDDTVHAFVGLAAIQIAQIDMLKAAGLQPDGILGHSVGELACGYADNSLSHEEAILAAYWRGRCVKEAKLPPGGMAAVGLTWEECKQCCPPNVVPACHNSEDTVTVSGPLDSVNEFVAKLKKDGVFAKEVRSAGVAFHSYYMASIAPVLLSALKKVIPHPKPRSARWISTSIPESQWQSDLARNSSAEYHVNNLVNPVLFHEGLKHVPENAVVVEIAPHALLQAILRRTLKPTCTILPLMKKEHKNNLEFFLTQTGKIHLTGINVLGNNLFPLVEYPVPVGTPLISPYIKWDHSQDWDVPKAEDFPSGSKGSASASVYNIDVSPDSPDHYLLGHCIDGRVLYPAAGYLVLAWRTLARSLGMAMEQMAVTFEEVTIHQATILPKKGSVQLEVRLMPASHCFEVSGNGNLAVSGKISLLENTALKNFHNQAVDFQTQVDTSSKSGLLKEDVYQELHLRGYNYGPTFQGVLECNNEASTGKVVWNGNWVTFLDSLLHVIILAETGRSLRLPTRIRSVIIDPVLHQEQVYQYQDNVEAFNVTVDHCLNSLKAGGVQINGLHASAAPRRQQERNPPTLEKFCFVPYTESNCLSSSVHLHAYLGHCKDLIQNLQAKVAVHGVKLVIPGLETAVAAAESSPIQKGLQHILAAICHLELNGNLHSELEQIVTQEKMHFQDDPLLNGLLDSSELKTCLDVALENMTSHRMKIIEALAGSGHLFSRVKSVLNTQPLLQVDYIATDCSLEALSANETELQDAGVSLSQWDPSSLPSGNLTNADLVVYNYSTSVLGNTTEILSNLAAAVKEGGFVLLHTLLKGETLGEIVSFLTSPDLQQKHSFLSEAQWEGLFSKASLNLVAMKKSFFGSVIFLCRRQAPAKTPVFLPIDETHFKWVESLKEVLADPSEQPVWLTATSCGNSGILGMVNCLRLEAEGHRIRCVFVSNLNRSSAVPPMTLSSLEMQKIVQRDLVVNVYRDGKWGSFRHLPLQQAQPQELTEYAYVNVLTRGDLSSLRWIVSPLRHFHATNPNVQLCKVYYASLNFRDIMLATGKLSPDAIPGNWALQQCMLGMEFSGRDLAGRRVMGLLPAKGLATVVDCDKMFLWEVPKNWTLEEAASVPVVYATAYYALVVRGGMRKGESILIHSGSGGVGQAAIAIALSMGCRVFTTVGSTEKREYLQARFPQLDANSFASSRNTTFEQHILRVTNGKGVNLVLNSLAEEKLQASLRCLAQHGRFLEIGKFDLSNNSRLGMALFLKNVAFHGILLDAIFEDGNKEWEVVSELLKKGIKDGVVKPLRSTVFNKEEVEAAFRFMAQGKHIGKVMIKVQEEEKENPLRRSEPVKISAISRTLCPPTKSYIITGGLGGFGLELAQWLVERGAQKLILTSRSGIRTGYQAKRVREWKALGIQVLVSTSDIGTLEGAQLLIEEALQLGPVGGIFNLAVVLRDGMIENQTPELFWEVNKPKYSGTLHLDWVTRKKCSDLDYFVVFSSVSCGRGNAGQSNYGFANSTMERICEQRHHDGLPGLAIQWGAIGDVGILLKTMGNKEIVVGGTVPQQISSCLEVLDIFLNQPHPVMSSFVLAEKISLKTEGGSQRDLVEAVAHILGVRDVNSLNAESSLADLGLDSLMGVEVRQTLERDYDIVMTMREIRLLTINKLRELSSKSGTAEELKPSQLMKTGPGEPPKLDLNNLLVNPEGPTLTRLNDVQSTERPLFLVHPIEGSIAVFYTLASRLHVPCYGLQCTKAAPLDSIQSLATYYIDCMKQIQPEGPYRIAGYSFGACVAFEMCSQLQAQQNASHALNSLFLFDGSHSFVAAYTQSYRAKLTQGNEAALETEALCAFVQQFTGIEYNKLLEVLLPLEDLEARVNAAADLITQIHTNINREALSFAAASFYHKLKAADKYIPESKYHGNVTLMRAKSHNEYEEGLGGDYRLSEVCDGKVSVHIIEGDHRTLLEGDGVESIIGIIHSSLAEPRVSVREG

SEQ ID NO: 18 - protein, 2515 aa, Phaethon lepturus
MEDVVIAGIAGKLPESENLQEFWENLFNGVDMVTEDDRRWKPGIYGLPKRNGKLKDISKFDASFFGVHPKQAHTMDPQLRLLLEVSYEAILDGGINPATLRGTDTGVWVGASGSEAGEALSQDPEQLLGYSMTGCQRAMFANRISYFYDLTGPSITIDTACSSSLVALENAYKAIRHGQCCAALVGGVNLLLKPSTSVQFMKLGMLSPDGACKAFDVSGNGYCRSEAVVVVLLTKRSMAKRIYATIVNAGSNTDGFKEQGVTFPSGEMQQRLVSSLYRECGIRPGEVEYIETHGTGTKVGDPQEVNGIANVFCQCEREPLLIGSTKSNMGHPEPASGLAALAKVTGGLRRKILGGSTTVMRCSLRDIPALQDGSLEVVCKPTPVKGGLVSINSFGFGGANAHVILRPNEKKRQPLETCNTPRLVQICGRTQEAVEILIQESRRHGGCSPFVSLLSDISTVPVSFMPYRGYTLVGTESDIKEVQQVQASDRPLWYICSGMGTQWKGMGLSLMKLDLFRQSILRSDEALKNTGLKVSDLLLQADENTFDDTVHAFVGLAAIQIAQIDMLKAAGLQPDGILGHSVGELACGYADNSLSHEEAILAAYWRGRCVKEAKLPPGGMAAVGLTWEECKQCCPPNVVPACHNSEDTVTVSGPLDSVNEFVAKLKKDGVFAKEVRSAGVAFHSYYMASIAPVLLSALKKVIPHPKPRSARWISTSIPESQWQSDLARNSSAEYHVNNLVNPVLFHEGLKHIPENAVVVEIAPHALLQAILRRTLKPTCTILPLMKKEHKNNLEFFLTQTGKIHLTGINVLGNNLFPLVEYPVPVGTPLISPYIKWDHSQDWDVPKAEDFPAGSKGSASASVYNIDVSPDSPDHYLVGHCIDGRVLYPATGYLVLAWRTLARSLGTAMEQTAVMFEEVTIHQATILPKKGSVQLEVRLMPASHCFEVSGNGNLAVSGKISLLEHTAPKNFHNQPVDFQTQADMSSKSGLLKDDIYQELHLRGYNYGSTFQGVLECNSEASTGKVLWNGNWVTFLDTLLHVIILAETGRSLRLPTRIRSVCIDPVLHQEQVCQYRDNVEAFDVVVDRCLDSLKAGGVQINGLHASVAPRRQQERIPPTLEKFCFVPYNEHNCLSSSVHLHAYLERCKDLIQNLKAKVAVHGVKLVIPGLETTVAAAESSPIQNGLQHILAEICRLELNGNLHSELEQIVTQEKMHFQDDPLLNGLLDSSELKTCLDVALENMTSHRMKIVEALAGSGHLFSRVKSILNTQPLLQVDYIATDHILETLSANETELQDAGVLFSQWDPSSLPSGNLTNADLVVYNCSTSVSGNTIEILSNLAAAVKEGGFVLLHTLLKGETLGEIVSFLTSPDLQQKHSFLNEAQWEGLFSKASLNLVAMKKSFFGSVIFLCRRQAPAKTPVFLPIDETHYKWVESLKEVLADPSEQPVWLTATSCGNSGILGMVNCLRLEAEGHRIRCVFISNLNPSSAVPPTSLSSLEMQKIVQRDLVMNVYRDGKWGSFRHLPLQQEQPQELTEYAYVNVLTRGDLSSLRWIVSPLRHFHATNPNVQLCKVYYASLNFRDIMLATGKLSPDAIPGNWALQQCMLGMEFSGRDLAGRRVMGLLPAKGLATVVDCDKMFLWEVPKNWTLEEAASVPVVYATAYYALVVRGGMKKGESVLIHSGSGGVGQAAIAIALSMGCRVFATVGSTEKREYLQARFPQLDADSFASSRNTTFEQHILRVTNGKGVNLVLNSLAEEKLQASLRCLARHGRFLEIGKFDLSNNSQLGMSLFLKNVAFHGILLDAIFEQGNQEWEVVSELLKKGIKDGVVKPLRSTVFNKEEVEAAFRFMAQGKHIGKVMIKVQEEEKEYSLRRFETVKISAISRTSCPPTKSYIITGGLGGFGLELAQWLVERGAQKLILTSRSGIRTGYQAKRVREWKALGIQVLVSTSDIGTLEGTQLLIEEALQLGPVGGIFNLAVVLRDGMIENQTPEFFWEVNKPKYSGTLHLDWVTRKKCPDLDYFVVFSSVSCGRGNAGQSNYGFANSTMERICEQRHHDGLPGLAIQWGAIGDVGILLKTMGNREVVVGGTIPQQISSCLEVLDIFLNQPHPVMSSFVLAEKVSVKTEGGSQRDLVEAVAHILGVRDVSSLNAESSLADLGLDSLMGVEVRQTLERDYDIVMTMREIRLLTINKLRELSSKSGTAEDLKLSQLMKTGPGEPPKLDLNNLLVNPEGPTITRLNDVQSTERPLFLVHPIEGSIAVFYTLASRLHIPCYGLQCTKAAPLDSIQSLAAYYIDCMKQIQPEGPYRIAGYSFGACVAFEMCSQLQAQQNASHAFNSLFLFDGSHSFVAAYTQSYRAKLTQGNEAALETEALCAFVQQFTGIEYNKLVEVLLPLKDLEARVNAAADLITQIHTNINREALSFAAASFYHKLKAADKYIPESKYHGNVTLMRAKSHNEYEEGLGGDYRLSEVCDGKVSVHIIEGDHRTLLEGDGVESIIGIIHSSLAEPRVSVREG

SEQ ID NO: 19 - protein, 2140 aa, Phoenicopterus ruber
EVVCKPTPVKGGLVGINSFGFGGANAHVILRPNDNKSQPLKTCNMPRLVQICGRTQEAVEVLIQESRKHGGCSPFLSLLSDISTVPVSSMPYRGYTLVGTESDIKEVQQVQASGRPLWYICSGMGTQWKGMGLSLMKLDLFRQSILRSDEALKNTGLKVSDLLLQADENTFDDTVHAFVGLAAIQIAQIDLLKAAGLQPDGILGHSVGELACGYADNSLSHEEAILAAYWRGRCVKEAKLPPGGMAAVGLTWEECKQCCPPNVVPACHNSEDTVTVSGPLDSVREFVAKLKKDGVFAKEVRSAGVAFHSYYMASIAPVLLSALKKVIPHPKPRSARWISTSIPESQWQSDLARNSSAEYHVNNLVNPVLFQEGLKHIPENAVVVEIAPHALLQAILRRALKPTCTILPLMKKEHKNNLEFFLTQTGKIHLTGINVLGNNLFPLVEYPVPVGTPLISPYIKWDHSQDWDVPKAEDFPAGSQGSASASVYNIDVSPDSPDHYLVGHCIDGRVLYPATGYLVLAWRTLARSRGMAMEQMAVMFEEVTIHQATILPKKGSVQLEVRLMPASHCFEVSGNGNLAVSGKISLLEDTALKNFHNQPVDFQTQVDMSSKSGLLKEDIYQELHLRGYNYGPTFQGVLECNSEASTGKVLWNGNWVTFLDTLLHMIILTETGRSLRLPTRIRSVCIDPVLHQEHVCQYQDNIEAFDVVVDRCLDSLKAGGVQINGLHASVAPRRQQERIPPTLEKFCFVPYTESNCLSSSVHLNAYLEHCKDLIQNLQAKVAVHGVKLVIPGLETAVAAAESSPIQKGLQHILAEICRLELNGNLHSELEQIVTQEKMHLQDDPLLNGLLDSSELKTCLDVALENTTSHRIKIVEALAGSGHLFSRIKSILNTQPLLQVDYIATDRILETLSANETELQDAGVSFSQWDPSSLPSENLTNADLVVYNYSTSVLGNTTEILSNLAAAVKEGGFVLLHTLLKGETLGDIVSFLTSPDLQQKHSFLSEEQWEGLFRKASLNLVAMKKSFFGSVIFLCRRQAPAKTPIFLPIDETHYKWVESLKEILADPSEQPVWLTATSCGNSGILGMVNCLRLEAEGHRIRCVFVSNLNPSSAVPPTSLSSLEMQKIVQRDLVMNVYRDGKWGSFRHLPLQQAQPQELTEYAYVNALTRGDLSSLRWIVSPLRHFHATNPNVQLCKVYYASLNFRDIMLATGKLSPDAIPGNWALQQCMLGMEFSGRDLAGRRVMGLLPAKGLATVVDCDKKFLWEVPKNWTLEEAASVPVVYATAYYALVVRGGMKKGESILIHSGSGGVGQAAIAIALSMGCRVFATVGSTEKREYLQARFPQLDASSFASSRNTTFEQHILRVTNGKGVNLVLNSLAEEKLQASLRCLARHGRFLEIGKFDLSNNSQLGMALFLKNVAFHGILLDAIFEDGNEEWEVVSELLKKGIKDGVVKPLRSTVFNKEEVEAAFRFMAQGKHIGKVMIKVQEEEKEYLLRKSEPVTISAISQTSCPPSKSYIITGGLGGFGLELAQWLVERGAQKLILTSRSGIRTGYQAKCVRQWKALGIQVLVSTSDIGTLEGTQLLIEEALQLGPVGGIFNLAVVLRDGMIENQTPELFWEVNKPKYSGTLHLDWVTRKKCPDLDYFVVFSSVSCGRGNAGQSNYGFANSTMERICEQRHHDGLPGLAIQWGAIGDVGILLKTIGNKEVVIGGTVPQQISSCLEVLDIFLNQPHPVVSSFVRAEKVSVKTEGGGQQDLVEAVAHILGVRDTSSLNPESSLADLGLDSLMGVEVRQTLERDYDIVMTMREIRNLTIKKLRELSSKSGTAEELKPSQLMKTGPGEPPKLDLNNLLVNPEGPTITRLNDVQSTERPLFLVHPIEGSIAVFNTLASRLHMPCYGLQCTKAAPLDSIQSLAAYYIDCMKQIQPEGPYRIAGYSFGACVAFEMCSQLQAQQNASHALNSLFLFDGSHSFVAAYTQSYRAKLTQGNEAALETEALCAFVQQFTGIEYNKLLEVLLPLKDFEARVNAAADLITQIHTNINREALSFAAASFYHKLKAADKYIPESKYHGNVTLMRAKSYNEYEEGLGGDYRLSEVCDGKVSVHIIEGDHHTLLEGDGVESIIGIIHSSLAEPRVSVREG

SEQ ID NO: 20 - protein, 2419 aa, Ooceraea biroi
MPAQFESINNPMTRESMARNGVPYSIDSDIVITGISGRLPESSNIEEFKENLMKEMDMVTDDERRWPAGIYGLPLRTAKLKDLDNFDASFFGVHAKQAHVMDPQLRMMLEITYEAIVDAGINPSTIKGSRTGVFVGVSTSESDEYWTKDTEKVNGYGLIGCCRAMFANRVSFAFDFHGPSFVLDTACSSSMVAMHEAVTAMRAGECDAAIVGGMNLLLKPANSLQFHKLGMLSSDGKCKAFDATGKGYVRAEALVAIYLQKAKDARRVYATVVHTKTNMDGYKFQGITYPNGEMQNQLMREVYSETGINPADIVYVEAHGTGTKVGDPEEINSVDQLFCKDRKTPLLIGSVKSNMGHSEPCSGLCSIAKVLIAMESGMIPANLHFSTPNPDIPALMEGRIRVITKATPWNGGLVGINSFGFGGANAHVILRSNSKPKLSPVLDTAKLLPKLVAVSGRTEDAVYTLLDKAKEHRQDDEFLSLLHAMHNNDIPGHGFRGYEILGIDHTRELTEVASYNEKRPIWFVFSGMGTQWSGMGRELLGIEVFRRTLQRCADTLLPHGVDLMNIILNNTNETLENVIDTFVAIAAMQVALIDVLAAIDIYPDGIAGHSVGELGCAYADGAFTAEQTVLAAYWRGKSIVDSTLEPGAMAAVGLSWEDAQKMCPPNVSPACHNSMDSVTISGPTKDVLKFVEELKSKNIFAKLVRSSGIAFHSKYIASAGPKLRASLDKIIPNPKQRSAKWISSSIPEAAWGSPLAQVSSSAYHVNNLLSPVLFQEAIAHIPDNAITIEIAPHCLLQAILRRSLPSTVTNVSLHMRDHTNNLAFLLSNIGKLYMAGAQPNISKLYPPVSFPVGRGTPMIGPLVKWDHSQKWNVASFKKTSGQSGECIVQVDLSKETDAYIAGHQIDGRILFPATGYMLLVWKTLAKLRGTDFELLPVVFENLRFQRATIMPKEGAIKFSINIFEGTGEFEISEAGTVVVRGNIRVSEAIEKDQLKLPPPAVPPVKEEILPLNTKDVYKELKLRGYEYHGIFQGINSCDNYATAGELRWSNEWVSYMDTMLQFSILARKHKLMYIPSRLQYCAINPVLHKQFVEGLPQDGGLPVYYYKNVNIVKSGGIEFRGLNASVVARRQQTQAEPRHERYIFVPYENPHSLVEDPTRGKLHALTILTQIMYENMTALKLKIVEIADERAAEALLTPLVTEIVNNELSSVTVEVQVAAVSPGNYAEAFNHVDVNVSIVAQDANTAPPAQDVHLGVAVDVLSNKSYTILKNLSASLKPSGFILLEETAAHLDLETALKDTDLTLVSRQTDSIGKNYLLLKKLKKSGEPIVIQITEKNLSWVETLKAALKITYTGEQEVLLMSQGEELIGLAGLITCIRKETGGANVRYVFIQDKNSPKFGLSVPYYVNQLDKGLMANVLKGGQWGSYRHLQLDRQNNVSSLQVEHAYVNTLTRGDLGSLRWIEGPLSYYQPDNSSNTMLCSVYYAPLNFKDIMMATGKLPADALPGKMAAEECVLGIEYSGRDENGRRVMGIVEAKGLATTVLTDLDFLWEVPDKWTLEEAATIPVAYATSYYALFVRGQLKAGESVLIHAGSGGVGQAAIAIALRTGCTVFTTVGTLEKRKYLKKRFPQLIDSHIGNSRDTSFEQMILTETQGRGVDLVLNSLAEEKLQAGIRCLAMNGRFLEIGKYDLSNDNNVGMSMFLKNTTFHGVLVDALFYASSLEKKELMRLVSEGIKNGAVQPLPSTVFTEQQVEQGFRYMASGKHIGKVLLKIREEEPKKCVSPAPKTVAAVPRTYMNPEKSYVLIGGLGGFGLELANWMIARGAKFIVLVSRFGITTGYQSLCVRRWRENGVKVVISTMDITTLSGAERLIAQSNQLAPIGGIFNLAAVLRDALIDNQKEADFKAVMLPKVDGTRNLDVVSRKLCPSLDYFVVFSSISSGRGNIGQTNYGFANSAMERMMEERQANGLPGLAIQWGAIGDVGLIIEKMGNSSEIGSTLPQHISSCLATMDIFLQQPHAVLTSVISAEKHKPNTDSNRISLVEAIANILGIKDIKLVNLNNSLADLGMDSLMGTEIKQTLERNYDIALSPQDIRSLTFSKLQELSVTSTTTMKEQQPVAANASTATNSLANAIENNAADNMLLMQWPSNEVLPKEVLVRMKTKSANGPALFIVHAIEGSVNALETLASEFERPVWGLQCVEQAPHDSILELAEFYVKAIRKVQKKGPYHVAGYSFGSCVAIEMAMQLEFAGEKVILSLIDGSPAYIRQQTEMIGKVDTTQEDYIPSDACMKALAYFSIQFNKQLNFFQAYSILKESKSETDMFNKMLQVIGDIPFAQDDLKIAGYLLFKKLTAAMRYSPDKKFKGPVTLIKATDNFLHLTKDYDLSQICVQPARIDEVSGNHRSILLGESAKRIASLLRM

SEQ ID NO: 21 - protein, 2453 aa, Anopheles funestus
MPARFEEVTATDTRRGVPIDLAGGHYDVDAGVGGHAGHRPNIVDDICITGFSGRLPESSDIGEFTRNLLEGVDMVNDDDRRWPAGLYDLPTRIGKIKDEDLQHLDAEFFKIHQKQAECMDPQLRMLLECTHEAIIDAGINPQEIRGSRTGVYIGCSNSETEQHWCADPDLVNGYGLTGCARAMFANRISYTFDFKGPSYAVDTACSSSLFALSQAFADMKAGHCDAAIVAGCGLILKPTMSLQFKRLNMLSKDGMCKAFDESGNGYVRSDGCVVTFLQRASNSRRIYASVLNVRTNTDGFKEQGITYPIGEMQKRLIRETYEEIGLNPADVVYVEAHGTGTKVGDPQEVNAITDFFCKDRKTPLLIGSVKSNMGHSEPASGVCSIAKILIAMEQGVIPGNLHYKNPNPDLYGLLDGRLKVVDRNLPWNGGIIGLNSFGFGGANAHVIMKSNPKPKPISPKDDGFPKLMVASGRTTEAVEAFLDQATVSKDDEEFVGIVNEIHSRNIPLHNYRGYTVVAGDGQSQTVREVLEVTADDKRPVWFIYSGMGSQWASMAKDLMQLEVFHNSIYRCAEALRPEGVDLIDVLTKSDDTKFDNILNSFISIAAVQVALTDVLTHVGITPDGMVGHSVGELGCAYADGCFTPEQTVLAAYWRGRSILDTDLIAGQMAAVGLSWEECKQKLPKDVIPACHNSADSVTISGPVNSVGKVIADLNAQGIFAKGVKSSGIAFHSRYIADAAPKLRKSLDKIIPNPKNRTPRWISTSIPEESWPTPLAQQSSSAYHVNNLLSPVLFSEGLKHVPANAICIEIAPHGLLQAILKRALGKDATNLSLMKRDHANNMIFLLSNLGKLYAAGVQPQVQKLYRPITYPVGRGTPMLNSLVKWDHSINWFLARIGVENKSGETIIDVNLGKDEDAYLAGHTIDGRVLFPATGYLTLAWRTYAKMQGADIEKTPVVIENAVFHRATILPKDGSVKFGINFFDGTGAFEICEGGTLAVSGKLTIPEKIELEELPLNKLEADKSGLALNMSDVYKELRLRGYDYANMFRGVTRSDPRALTGELQWRDNWVSFMDTMLQFSILGKDLRELYLPTRIEKIVINPARHMELVNNLTQTGDDRTLPVYMYRDINVIKSGGVEMRGLRATLAPRRQGTQAPPTLEKYVFVPNNNEKELAEDNSEKARLRSITAAVHLVIENSSGALKIKVAEASFERSPENTMAGTVQAIIEGEPTLASDVAVVTNHQPDTLVQHYGESGVRVINKDAAAGPIEQNCHLAIGYDTFGRADPEAILCNLRDSIKSDGFVLLEESRSTFDARCRATVVKTGFTIISTQVCDKKVFVLLRPIVAELAQRKSIIITISERSFSWVEELKAALAKAEETATYVYIVCQGEELCGAQGLMNCVKNELGGKFARLFFIQDRKADKFSLTSTLYKEQLAKDLICNVLRPANGTPTAAWGCFRHLRLDNQSNAPSLPVEHAYINALTKGDLASLKWIESSLSRDRPDAAARNDRTELCTVYYAPINFRDVMLSSGKLGADALPGDLATQDCILGLEFAGRDSNGRRIMAMVQAKSLATTCVAQRNMIWEIPDNWTMEQASTVPCVYSTVYYALVMRGRMKRGESILIHAGSGGVGQAAISVALAAGVTVYTTVGSKEKREFLKRTFPQLTDRNIGNSRDCSFEQLVMRETQGRGVDLVLNSLAEEKLQASIRCLGLNGRFLEIGKFDLNNNSPLGMSVFLKNTSFHGILLDSVMEGDDDTIAEVVKLVADGIKSGAVRPLPTSVFTEQQVEQAFRFMASGKHIGKVVVRVREEEKAKIVKPSPKLINAIPRTYMHAEKVYILIGGLGGFGLELSNWLVSRGAKIIVLTSRSGVRTGYQSLMIRRWTERGVKVVIDTNDVTTLKGAQKLLTDAARHGPVGGVFNLAAVLRDGLLENSSEADFRAVCVPKVDGTKNLDVATRELCPDLDYFICFSSVSCGRGNQGQVNYGLANSAMERICEARHAAGLPATAIQWGAIGDTGLVLENLGDNDTVVGGTLPQRMPSCLQTMDLFMQQPSPVLASMVIAEKRKTDTGGVSLVGCIANILGLKDTKNVSDGATLADLGMDSLMGAEIKQTLERSFDLVLSAAEIRLLTFGKLRSFEKGGSGGDAGAASGAPAAAAAGAGGKRTDEHAIGDGTQVKFSADLMPKECLVRLESAVPAGSKARPVFAVHAIEGVITALIPLAQTLPVPVYGLQCVEAAPLDSLETLAAYYIKQIRTIQARGPYTIIGYSFGASIAYEMVVQLEKAGDACHLLLLDGSPRYVSWYTEAQKQRNANGEVVQAVDEAYALAYFAMVCGSLDYGRTAKELVEAKSWDDRVAKCAEMVHAKIPQYSRQLLATTARSFVGKIVASHMYKPASKIKATVKLVKPTENYAKLQGDYGLSDLCQQKVEIFTVQGDHRSMLIGDSMKQIASVLQQLF

SEQ ID NO: 22 - protein, 2469 aa, Anopheles darlingi
MPARFEEVTATDTRRGVPIDLAGHYDDHHHDTGSNIGNNHHQQQQHGRRPAIRDDICITGFSGRLPESSNIDEFRRNLFEGVDMVNDDDRRWPSGLYDLPTRIGKIKDEDLQHLDAEFFKIHQKQAECMDPQLRMLLECTHEAIVDAGINPQEIRGSRTGVYIGCSNSETEQHWCADPDLVNGYGLTGCARAMFANRLSFTFDFKGPSYAVDTACSSSLFALSQAFADMKAGHCDAAIVAGCGLILKPTMSLQFKRLNMLSKDGMCKAFDESGNGYVRSDGCVVTFLQRASDSRRVYASVLNVRTNTDGFKNQGITYPIGEMQKRLIRETYEEIGLNAADVVYVEAHGTGTKVGDPQEVNAITDYFCKDRKTPLLIGSVKSNMGHSEPASGVCSIAKILIAMEAGVLPGNLHYRNPNPDLYGLLDGRLKVVDRNMPWNGGIIGLNSFGFGGANAHVILKSNPKPKPISPRDDAFPKLVLASGRTTEAVEAFLDQTAISKDDEEFVGLVNEINSRNTPLHYHRGYTVVSGDGKPPVREVLEVSDEKRPLWFIYSGMGSQWASMARDLMQLEVFHSTIARCAEALRPEGIDLIDVLTKSDESRFDNILNSFISIAAVQVALTDVLTHVGIVPEGMVGHSVGELGCAYADGCFTPEQTVLAAYWRGRSILDTDLIAGQMAAVGLSWEDCKQKLPKDVIPACHNSSDSVTISGPVASVGKVIADLNAQGIFAKGVKSSGIAFHSRYIADAAPKLRKSLDKIIPNPKPRTQRWISTSIPEESWGTALAQQSSSAYHVNNLLSPVLFAEGLKHVPANAICIEIAPHGLLQAILKRALGKEATNLSLMKRDHDNNLIFLLSNLGKLYAAGAQPQVQKLYPPITYPVGRGTPMLNSLVKWDHSDKWFLARIGVENKSGETIIDVNLGKDEDAYLAGHTIDGRVLFPATGYLTLAWRTFAKMRGADLEKTPVVIENAVFHRATILPRDGSVKFGINFFDGTGAFEICEGGTLAVSGKLTIPEKIELEELPLAKLEADRSALPLNTSDIYKELRLRGYDYGGMFRGVTRSDSKALTGELQWRDNWVSFMDTMLQFSILGKDLRELYLPTRIEKIVINPARHLEIVSGLPVAKDGTDRTLPVYMYRDINVIKSGGVEMRGLRATLAPRRQGTQAAPTLEKYVFVPNSNEKELAENADKARLRSITSAVHLVIENSAGALKIKVVEASAERSPENTMAGTVQGIIEGEPTLASDVAIATTHQPDSLVQHYGESGVRVLNKDIAAGAIEQGCHLAIAYDVFGSGATADVLRNLRDTIKTEGFVLLEESRTGIDLSAKGAVRNALSKAGFLLVSEQVCDKKVFLLVRPILAEIAQRKSTIVTITEKNFSWLDSLKTALAKAEESSTYVYVVCQGEELCGAQGLLNCIKNEAGGKFARLFFIQDRSAEKFSLTSALYREQAAKDLICNVLRPANGSPACWGTFRHLRLDSQSNAPSLPVEHAYINALTKGDLASLKWIESSLSRDRPAGDDRTELCTVYYAPINFRDVMLSSGKLGADALPGDLATHDCVLGLEFAGRDSSGRRIMAMVQAKSLATTCVAQRNMIWSIPDSWTMEQASTVPCVYSTVYYALVMRGRMKRGESILIHAGSGGVGQAAISVALAAGVTVYTTVGSAEKRAFLKRTFPQLTDRHIGNSRDCSFEQLVMRETQGRGVDLVLNSLAEEKLQASVRCLGLNGRFLEIGKFDLNNNSPLGMSVFLKNTQFHGILLDSVMEGDDETIAEVVRLVADGIANGAVRPLPTSVFSETQVEQAFRFMASGKHIGKVVIRVRDEEKQKVLKPAPKLINAIPRTYMHAEKAYILIGGLGGFGLELSNWLVSRGAKILVLTSRSGIRSGYQALMVRRWTERGVKVVIDTNDVTTLKGAQKLLTEAARHGPVGGVFNLAAVLRDGLLENSSEADFKAVCVPKVDGSKNLDQATRELCPDLDYFVCFSSVSCGRGNIGQVNYGLANSAMERICEARHAAGLPATAIQWGAIGDTGLVLENLGDNDTVVGGTLPQRMPSCLQTMDLFMQQPCPVLASMVIAEKRKTETGGVSLVSCIANILGLKDTKNVSDASTLADLGMDSLMGAEIKQTLERSFDLVLSAAEIRLLTFGKLRSFEKGGITSDGAAGGDGAAADGGSGAGGAAAKSAADQQTAIGDGTQVMFSAELMPKECLVKLESAAPPSKSPARPVFVVHAIEGVITALIPLAKTLPVPVYGLQCVEAAPLESLVVLAGYYIQQIKTVQPRGPYTIVGYSFGASIAYEMVAQLEAAGEKCSLLLLDGSPRYVSWYTEAQKQRNANGEVVQAEDEAYALAYFAMVCGKLDYGKTAHELVSAKSWNERVAKCAEMVHAKAPQYSKQLLETTARSFVGKIVASHMYKPSSKINANVKLVKPTENYAKLQGDYGLSDLCKQKVEILTVKGDHRSMLTGESMKQIAAALHQLC

SEQ ID NO: 23 - protein, 2540 aa, Glossina austeni
MYISVDFKSNDQIFILLHGAKANKINPSQVNHNRQHTNNNSNNENKNDSQKSSNKGKSANRRRKRRQKNSQNSNFSENPQQNHQDDVAQIVVKDNPSTSFPFHFSSSNSNYDSSSFPLVNGRLHKDSIMPARFVENSEPSVPMNLENRYDHPSSNGVNSDIVITGISGRLPESTNIEEFRKNLFEGVDMVNDEPRRWSKGLYGLPERNGKIKEEDLENFDQTFFGIHQKQADGMDPQMRMLLECTYEAIIDAGLNPAEIRGTRTGVYVGASSADSENYQLSDPDRVDGYGLIGCARAMFPNRISFSFDLKGPSYCVDTACSSSLYALSQAFADMNSGKCDAAIVAGSNLCLRPTMSLQFKRLSMLSAEGKCKAFDESGSGYVRSDAVVVILLQRVSVAKRVYATILNARTNVDGHKEQGITFPNGQMQNQLMRETYGEINLDPAEVNYVEAHGTGTKVGDPQEVNSITDFFCKNRKSPLLVGSVKSNMGHSEPSSGLCSIAKVLLAMEQNVLPPNLHYKNPNPDLYGLLDGRLKVVDKPTPWEGGIVGINSFGFGGANAHVILKSNPKPKVITPTVGPPKLILCSGRTSDAVQEFLEDAEQHKDDDEYLTLINDIHSRPISGHYYRGFSVMDSKGALQKDVAEINPESRPIWFVYSGMGSQWASMAKDLMVFDTFKKTIHRCCEILRPEGLDLMEVLTRSTEKTFENILNSFVGIAAMQVALTDLLTSLNIVPDGIVGHSVGEVACAYADGCLSMEQTILAAYWRGKSILDTKLTKGKMAAVGLSWEEAHKRLPADCFPACHNSAENCTISGPEESIDAVCQKLTAEGVFARAVKSSGYAFHSKYIADAGPKLRKSLEKVIPNAKNRSPRWISSSIPEVAWNTAIAQQASAAYHVNNLLSPVLFHQALQHVPKNAICIEVAPTGLLQAILKRSLGNETTNLSLIKRDYENNPEFFLASIGKLYAAGAQPQIMTLSKPISYPVGRGTPMLGCKVGWDHSQKWHVPKFDKMTSSGETIIEVDLSKEEDAFLAGHTIDGRILFPATGYMTLAWMTYAKMRGHEFQKTPVVMENVVFHRATILNKEGVVKFGINFFDGTGAFEICEGGSLAVSGKISIPENVDNEQLPLDPLPVSTVAKELSTSDVYKELRLRGYDYAGIFRGITASDSLSSTGKLQWVDNWISFMDTMLQFSILSKNLRELYLPTRIEKAIINPDHHLKEIAKLTPEQQTKEGVPVYMYDDINVIKSGGIELRGMKASLAQKRPSNQNPPTLERYAFIPNENGVDLNENHEKAHFHALSVALRLLNENSSGALKIKGVELADGRNPDSLLAAKIVQVLEGEPSLTAEIAVATSNSNEEAISAALGPDAGVRVISKNILKEPVDQNNHFVFGVDVLSRPDTAVLVNSLASIKDNGFLIFEESAYGYRNTGRNLLTEFGLVVASEQSCGNSRVLVMARRPVDLKQRKSVVINVTEKNFDWLEELKAALAKAAENEQYVYIVCQGEEAFGAVGLMTCIRRENGGNFARLVFVQDGNAKPFSFNDAFYTKQLAKDLISNVLKNGLWGTYRHMKLETNAATLQVEHAYVNALVKGDLASLKWIESPRLTPNISNDFELCTVYYAPINFRDVMLSSGKLSADALPGDLAQQDCVLGLEFSGRDSKGRRIMAMVPAKSLATTCVAAKIMTWQIPDKWSMEEASTVPCVYSTVYYALMVRGQMKKGEKILIHAGSGGVGQAAISVALHHGLTVFTTVGSKEKREFLKKRFPQLKDNHIGNSRDTSFEQMIMRETQGRGVDLVLNSLAEEKLQASVRCLGLNGRFLEIGKFDLSNNSPLGMSVFLKNTSFHGILLDSVMEGEEAMQKQVVDLVAEGIKNGAVRPLPTSVFNDQQVEAAFRFMASGKHIGKVVVKIRDEEPGSKAIKPPTHLVNAIPRTYMHPEKSYVLIGGLGGFGLELTNWLVFRGAKHIVLNSRSGLRTGYQALMVRRWQEKGVKVLVDTSDVTNASGCKQLLTNANKLATVGGIFSLAAVLRDDILENQTAKDFKTVCDPKVTATKLLDQCSRSLCTSLDHFVCFSSVSCGRGNIGQSNYGLANSAMERICEARQASGFPGLAIQWGAIGDTGLVIETLGDNDTVIGGTLPQRMNSCLQTIDLFLQQPHPVLASMVVAEKRKSDQSGGVSLIASVANILGIRNIKNIPEKSSLADLGMDSLMGAEIKQTLERNFDIVLSAQEIRQLTFGALTQMDSGPARAESATSPQATSPSAFGDGTQDVFSSELMPTETIVRLKSKAPADSNKRPLFIASPIEGFADPLKNLAELLDCPVYGLQCTANADLDTIQSLASFYLKEVRKIQAKGPYVIAGYSFGAAVAFEMVINLQSHDIVSNYIALDGAPKYVSWNTSSFKDRRSMDDGDNYDYGLAYFAMVVANMDYAFVVKMLLDKPTWQEKLAKCAEMVAQEIKKPIDLIKQAGEIFYKKLSAGHNYKATTKITCPVTLVKPSENYVQLEEDYGFNEICQKPVKVFTVEGNHRTFLVEEASLKTIESELNRLTA

SEQ ID NO: 24 - protein, 2540 aa, Drosophila melanogaster
MPLQLNLLQAVDQPHPVAFKPNNCSTHQPQHSPSTQNNGKSARANRRRRRRRNKQQQNSQQPDAFRDTDAQLTPSTSSSTPHSAPNDRQDHLNSQFNPESINMPARFAEEVITAEPAQRAAPQLDLGGGHYVPRQQHLNDEIAITGFSGRLPESSTIEEFKQNLFDGVDMVNDDPRRWERGLYGLPDRIGKLKDSDLENFDQQFFGVHQKQAECMDPLLRMLLELTHEAIIDAGLNPSDLRGSRTGVYIGVSNSETEQHWCSDADRVNGYGLTGCARAMFANRISFTFDFKGPSYSIDTACSSSLYALEQAFSDMREGKVDNALVAGAGLILKPTMSLQFKRLNMLSPDGSCKAFDESGNGYVRSDGCVVLLLQRTSAARRVYASILNVRTNTDGFKEQGITYPIGKMQNRLIRETYEEIGLNPADVVYVEAHGTGTKVGDPQEVNSITDFFCKDRTTPLLIGSVKSNMGHSEPASGVCSVAKILIAMEEGVIPGNLHYNKPNPDLYGLVDGRLKVVDRNLPWNGGIIGLNSFGFGGANAHVILKSNPKPKALTPKDGALKVVLASGRTFEAVEQLLESASTNADDDEYLQLINEIHSKAIPNHFFRGYGVVSSKGTHQREVIESNDDKRPIWYIYSGMGSQWASMAKDLMKIEAFAKTIQRCADVLKPEGVDLIDVLTRSTDKSFENILNSFISIAAMQVALTDLLSSLGIHPDGIVGHSVGELGCAYADGCFTPEQTVLAAYWRGKSILDTQLAKGKMAAVGLSWEDAHSRVPSDCFPVCHNSEDNCTISGPEASIEALVAKLNAEGVFAKAVNSSGYAFHSKYIAEAGPKLRKSLEKIIPNAKNRTARWISTSIPESAWNTPVAKQSSAAYHVNNLLSPVLFHEALQHVPKNAISVEIAPHGLLQAILKRALGPDATNLSLVKRGHENNVEFFLTNVGKLFAAGAQPQVLTLVRPISYPVGRGTPMLNSKVGWDHTQKWLVAKFGKETSSGETIVEVDLSKEDDAFLAGHTIDGRILFPATGYMTLAWQTFAKMQGSEFHKTPVVMENLVFHRATILNKNAVVKFGINFFDGTGAFEICESGSLAVSGKITIPESIDNEELPLEEQTPSAVAKELGTNDVYKELRLRGYDYGGIFRGIVRSDTVASTGKLQWVDNWISFMDTMLQFSILSKNLRELYLPTRIERAVINPAKHFELLSALTKEEQVETGLPVQWYSDINVIKSAGVELRGLKANLAQRRPGTQAPPTLERYQFVPNINTTDLNENSEKARLHALDVAIQVIIENSSGAVKLKGVELANGRNPDVLVANRLLQIIEGEPVLTGDVAVVTSNNNEETITAALGDSGVRVVSKDVLKEPVEQNCHFVFGIDVLSRPDTKTLENSIASIRENGFLILEETLPTYTKTGRALLTKFGFVAVQEQSLGATRVLVLARKAVDLKTRKSVVVVATEQNFNWVDDLKAALATAATEEQYVYVVCQGEELFGAVGLMTCIKNENGGKLARLVFVQDAKAEKFSLTSTLYRQQLEKDLISNVLKNGAWGTFRHLKLETQQATLQVEHAYVNALVKGDLASLKWIEAAQADTAATVDKNLETCTVYYAPINFRDVMLTSGKLAADALPGDLAEQDCVLGLEFAGRDTQGRRVMAMVPAKSLATTCVASKRMMWQIPEKWTMEEASTVPCVYSTVYYALVVRGQMKKGEKILIHAGSGGVGQAAISVALAHGLTVFTTVGSKEKREFLLKRFPKLQERNIGNSRDTSFEQLVLRETKGRGVDLVLNSLSEEKLQASIRCLGLNGRFLEIGKFDLSNNSPLGMSVFLKNTSFHGILLDSVMEGEEEMQNQVVSLVAEGIKTGAVVPLPTSVFNDQQVEQAFRFMASGKHIGKVVIKVRDEEAGKKALQPKPRLINAIPRTYMHPEKSYILVGGLGGFGLELTNWLVTRGARYIVLTSRSGVKTGYQGLMIRRWQERGVKVVIDTSDVTTAAGAKKLLENSNKLALVGGIFNLAAVLRDALIEDQTAKDFKTVADPKVTATKYLDQFSRDICTELDYFICFSSVSCGRGNIGQTNYGLANSAMERICEQRQVSGFPGTAIQWGAIGDTGLVLENLGDNDTVIGGTLPQRMPSCLQTIDLFLQQPHPVVASMVVAEKRKSDQSAGVSLIATIANILGLRDTKNIQDGASLADLGMDSLMSAEIKQTLERNFDIVLSAQEIRQLTFGALKAMDGGADVKPAAAAPAAAAGVPEANITSGGSSRTASPMGDGTQVVFTTSLIPTEAIVQLDTKAPANSKQSPIFFISPIEGFASALEPLAKRLEVPAYGLQYTEAVPSDSLESAAKFFIKQLRTVQPKGPYKLAGYSFGCLLTYVMAGILEETNEVANVIMLDGAPSYVNWYTSSFKQRYTDGTNADNDNQSYGLAYFGIVLANIDYKALVRLLIVIPTWEEKLERFAELMSNEITQPVETIKKSATLFYKKLELADGYQPTLKLKTNVTLVKPTDNSAKLDEDYRLKEVCTKPVEVHTVEGNHRTFLIEDQSLKTIQSILKRLFN

SEQ ID NO: 25 - protein, 2574 aa, Brugia malayi
MNSSGEVPFWRDQEEIVITGISGRFPRCENVQEFGDLLLAGEDLVTEDSLRWPPGVFDLPKRHGKLKDLKKFDAQYFGVTPKQANYLDPQVRKLLEVTLEAIIDAGLNPTELRGSRTGVFVGCSASETGGVLTQDPETVTGYTLTGCVRSMFSNRLSFTFDLRGPSFSVDTACSSSLCALQLAVDAIRQEQCDAAIVAGAHLTLTPTAALQFLRLGMLSDKGSCRSFDASGDGYCRTEAIAAILLQRQSAARRIYATVVHAKSNTDGYKEQGITFPSGERQAALLEEVYREAGVDPNTVTYVESHGTGTKVGDPQEANAICQVFCQNRNSALLIGSVKSNMGHAEPASGLCSIAKVLIAMERGFIPPNLHYNEPNPYIPGLTDGRLQVVTEKTPFQRGLVGINSFGFGGSNTHIILRPANHSERKPAPTTFMKIIPYSGRTAEAVNNILDCVLAHPENAYLQQLLANQANLPAKDTPYRGYVIINQLPPLRDVQKIMITEPKQIYFIYSGMGSQWAGMVRQLMSIPAFDESLRSSSDAIVDFGVNVYEMLQSDDPSFYKNNTLNCMLAITAIQIALTDVLFLLGVMPDGIIGHSTGEMCCGYADGGLTREQTMQLAYHRGHTIMNANIKGGMAAVGLSWEEAGKRCPEGVIPACHNAADSVTISGDAEKIKEFVEELKKEDIFGKLVDSSGIPFHSPAMLKVKDKMLKAMRTSVPNPKPRSSRWISTSIPESNWENELAQMCSADYHTNNAVSPVLFYEALQKIPANAVTIEIAPHCLMHSILRRSLQKTCTNVGLINMKEKDRELESFLQALGKIYQTGITIHIEALYPAIQYPVPIGTPMISPMWRWDHSQDWPVVDGKTLSAAGGRQLPASYCYTVDPFASDSKETFLLDHIIDGRVLYPFTGHMVLAWKTIAKLNGVDFLKTPVILEEIRVYSATIVTKPIRLDVIVTPGNGYFEILDGDQLAASGYIRIVDEDVPFYYKSIQEIQTSEIAERIELDTEDAYKEFLLRGYEYGQAFRGIYRACNSGERGMLYWTGNWVTFLDSLLQTALLAERADSLRLPTRVRYLRIDPVKHMEHIQERDGIQVIELRNDVATNGCIAGGVECCDLTAHTVARRLQSSGQLYYEKIYFTKHFDMKAFDEFPQIREELNAYRDFLRSLLANGLAKWEVNGCLKELTNGALLSGAVRKFTKSPVATVFDEIFAIEIGNNPKDFENKVAEKMQSMLKIFNVDRLWSAAIVHDRILKTIQDTCIENSTGHNCKACALEFNSTEQLKFCVDAVNSHPLLEVEWLCVGPKVDDMDESTLVQLGVKKVTTVLDEKQFVPSAEIKNCDIIILDKILSQKKDVVRYLSRCKEMLRDDGFIILVETTSDYEIALAIQGLNAETISISDSGRIYGAYFTHDQLLKLFEECDFRLCNYQSDPSMMTTMYAIRKIPSQPREPIVIDVDDIKEFTWIEPLQKAIEERLNEPDYKTIWLTSTTVRNNGLLGLALCFNEENLKSNRFRTLIDMSVKKENRTGPAQIGMEHESVKQLVKLDLHANNYRDGVWGSMRHLVVKDDEMHLYKDVEHAFINTLIRGDVSSLTWFESPNQFFEDTCQKNPSLELCNVYYSAINFRDVMLAYGRLPPDAIPGQFADRECLLGMEFSGRLKDGTRLMGILPAQALATSVIVNREYAWTVPDNWSLTDAATVPVVYTTAYYALVMRGRIRRGDKVLIHGGSGGVGQAAISVALSRGCEVFTTVGNAEKKAFLQKRFPQLKDRHFANSRNSDFELHIRHQTRGRGVDVVLNSLAQHMLQASIRCLAQNGRFLEIGKVDLSQNSPLGMAVFLKNITFHGILLDAVMDPSVGKKEDWQEVARLVQDGIKSGIVQPLSYTTFSSEKAEEAFRFMSSGKHIGKVLMEIRREEPERVCVPSPIKVRAICRTLCHPNHVYLITGGLGGFGLELSQWLINRGAKKLVLTSRMGIRTGYQARCVHFWRRTGISVLVSTLNIAHLSDAHELISQCKALGPVGGVFHLAMVLRDCLFENQNVQNFKDAAEAKYWGTLNLDAATRDACGKELRWFVAFSSITSGRGNAGQTNYGWSNCTMERIIEHRREDGFPGIAIQWGAIGDVGVILENMGDNNTVVGGTLPQRMPSCLAALDLFLSWNHPIVASYIKADIGAKKVGSSGNLWQTMAHILGVNDAAQLNPDAYLGDLGLDSLMGVEIKQALERDYDIVLSMKDIRTLTLNKLQQLVDSGGTTSTVLQTTELELKKESEKEAEQNTVEQLERQMNQLFKMRVDVNDLEPMDLIIKCNKVEAGPPTFFVHSIEGIATPLKKVMSKCNFPAYCFQSTREVPQDSIEKVAQKYILEMKKIQPEGPYRIIGYSYGACIGFEMATMLQESDGANSVERLILLDGSHLYMQTYRNVYRMAFGVTGDTLVNNPLFESEIMCAMTLRFANVDYKKFRVELLQQPGFKARVQKVVDTVMTTGLFKSADTIDFACCAMRSKFVMADKYKPERKFKGLITLIRAEQGAAREEDVGSDYGISQVSDENKVYIVEGDHDSFVQGKTSAKTVNIINDLIAEANKKAKNV

SEQ ID NO: 26 - protein, 2613 aa, Caenorhabditis elegans
MDNTGEGSSDSSGTWERISDNSQSSDNMDPTQWWQKQDDIVISGVSGRFPRCDNVKMFGDMLLAGEDLVTEDSLRWTPGFCDLPKRHGKLKVLNKFDAGFFQVTPKQANFMDPQVRLLLEASWEAMVDAGINPTDLRGSKTGVFVGCSASETSGMLTQDPDTVTGYTLTGCVRSMFSNRISYTFDLQGPSFSVDTACSSSLLALQLAVDSIRQGQCDAAIVAGAHLTLTPTAALQFLRLGMLTDKGSCRSFDESGDGYCRTEGVAAIFIQRKKKAQRLYATVVHAKSNTDGHKEHGITFPSGERQAQLLQEVYSEAGIDPNSVYYVEAHGTGTKVGDPQEANAICEVFCSKRTDSLLIGSVKSNMGHAEPASGVCSLTKILLSIERQLIPPNLHYNTPNQYIPGLTDGRLKVVTEPTALPGGLIGINSFGFGGSNTHVILKAADHIAPPITPHPFTKLVTYCGRTQEAVENIFTEIESNKDDLYLQALLANQANMPANLLPFRGYMLLDRENNVETLKSITKVPITEARPIYFIYSGMGSQWPGMAIKLMKIPMFDDSLRASSKTLEEFGLDVYGMLCNPDPEQYSNNTMNCMLAITAIQIALTDVLTALGVSPDGIIGHSTGEMGCGYADGGITREQTMRLAYHRGTTIMKHTEIKGAMAAVGLTWEQVKEQAPPGVVAACHNGADSVTISGDAEGVATFCAQLKEKDIFAKVVDTSGIPFHSPAMLAVQDEMIECMRTAVPEPKPRSSKWISTSIPEDDWESDLAATCSAEYHVHNACSPVLFYEAIQKIPANAVTIEMAPHSLMQAILRRSLQKTVTNVGLMNRPKSENDDELESFLGSLGKIYQAGVNIQITELYPGGQYKGVVPKGTPMIGPMWKWDHTQDWLTIDGRQVLAGGSGSVASSATYNIDPFATDSKETYLLDHVIDGRVLYPFTGHMVLAWRTLCKLKGLDYTKTPVVFENINVFSATILTKPIKLDVVLSPGNGYFEIISDDQVAASGRIYIPEDNQPFYYGKLEDIRTSEIADRIELDTEDAYKEFLLRGYEYGQAFRGIYKTCNSGERGFLYWTGNWVTFLDSLLQTALLAERSDTLRLPTRVRHLRIDPNKHLEHVVEKDGIQVIELRNDHSTNGCIAGGVECCDLNAHSVARRIQVSGQLYHEKIFFVPHFDHNCLSGHKKTSTILKDYSAVIKQQLYTGFSKWQSAGLLKKLKNGAQIVKALAVLKASQSDVVLDDTVTRFTHDGKCTVLHHIADMFKIEDCEDFEDRVAAKLKSVRGIFELDRLWAGAVLNDRIVKSLQDICIENSAGHHATMAAVDLVSTDQIRHCIEANSSHPLLETDYTCIGANVDHLDESTLEIIGGKKQKIDLENNFTGHGEVKNLDYVLLDKVISKKADPIAFIEACKHLIRETGFLLVVEVTSQYEIALAIEGLLGNEMVGDASRKYNQFFTHEQLLDMFKSTGFLICNFQSDPALMTTTYAVRRVSPIPRDPVFIDVDDVKEFNWIEPLQKVSEERLNEPDSKTIWLVSNKCRNNGIVGLGLCFVEENLKINRFRSAFDMSANKEIRDGPPVWNIGDEETKKIVELDLHANDYMDGQWGSMRHIVVKDEDVHVYKDCEHAFINTLTRGDVSSLTWFESPNQYFDSMVKSKATQELCSVYYAPINFRDIMLAYGRLPPDAIPGNFADRECLLGMEFSGRLKDGTRLMGILPAQALATTVMVDRDYAWEVPRDWTLAEASTVPVVYTTAYYALVRRGLMKKGDKILIHGGAGGVGQAAIAIALAAGCEVFTTVGSAEKREFLKNLFPQLQEHHFANSRSADFELHIRQHTKGRGVNIVLNSLANEMLQASLRCLARHGRFLEIGKVDLSQNSSLGMAKLLDNVSVHGILLDSIMDPTVGDLDEWKEIARLLEQGIKSGVVKPLHSHSFPADKAEEAFRFMSAGKHIGKVIMEIRPDEGTKVCPPSKISVRAICRTLCHPQHTYLITGGLGGFGLELAQWLINRGARKLVLTSRTGIRTGYQARCVHFWRRTGVSVLVSTLNIAKKSDAVELINQCTAMGPIGGIFHLAMVLRDCLFENQNVQNFKDAAEAKYYGTINLDYASREHCDKNILKWFVVFSSITSGRGNAGQTNYGWSNSCMERMIDQRRADGFPGIAIQWGAIGDVGVILENMGDNNTVVGGTLPQRMPSCLSSLDNFLSWNHPIVSSFIKAELGSKKNVGGGDLMATIAHILGVNDISQLNADANLSDLGLDSLMGVEIKQALERDHDIVLSMKEIRTLTLNKLQQLADQGGTGRTALQVNELEMKKDGERDAELNTAEMLEQQMNQLFKMRVDVNDLDPQDIIVKANKVEEGPITFFVHSIEGIATPLKKVMNKCEFPAYCFQSTKNVPQTSIEDVAKCYIREMKKIQPSGPYRLVGYSYGACIGFEMANMLQESDGRDAVERLILLDGSHLYMQTYRNVYRMAFGVTGDSLVNNPLFESEIMCAMTLRFANVDYKKFRFELLQQPGFKARVQKVVDQVMLTGLFKSPETVAFACEAMHSKFLMADKYKPRRNFGGHITLIRAEQGAAREEDVGEDYGVAAVSEDCEVLKVKGDHDTFVQGKSSSVTVEHINRIILQ

SEQ ID NO: 27 - protein, 2731 aa, Caenorhabditis briggsae
MDNTGEGSSDSSGTWERISDNSQVSLLPEFRLSVSPNLSTVLLRSGRGHHHLRFSSLLLFYLLPSLPRARIRIVFASLLLLFLNVFVRSADAFLFFFSFFVYLFPLSFFRSSDNMESTNWWQKQDDIVISGVSGRFPRCDNVKEFGDLLLAGEDLVTEDDLRWPPGFYDLPKRHGKLKELKKFDAGFFSVTPKQANFMDPQVRILLEASWEAMVDAGMFLSISHFIPLFGTETEIGKLCLSPDVPGSINPIDLRGSKTGVFVGCSASETSGALTQDPESVTGYTLTGCVRSMFSNRISYTFDLQGPSFSVDTACSSSLLALQLAIDSIRQGQCDAAIVAGAHLTLTPTAALQFLRLGMLTDKGSCRSFDDSGDGYCRTEGVAAIFIQRKKKAQRIYATVLHAKSNTDGYKEQGITFPSGERQAQLLQEVYSEAGVDPNSVYYVESHGTGTKVGDPQEANAICQVFCSSRSDSLLIGSVKSNMGHAEPASGVMSLTKILLSIERQMIPPNLHYNTPNQYIPGLVDGRLKVVTEPTPLPGGIIGVNSFGFGGSNTHVILKAADHVAPAITQHPFTKIVTYCGRTQESVEKIFGEIEANKDDLYLQSLLANQANMPANLLPFRGFMLLDRENNQAALKNITKVPITEPRPIYFIYSGMGSQWPGMAIELMKIPMYDESLRASSKTLEEFGLDVYGMLCNPDPEQYSNNTMNCMLAITAIQIALTDTLTALGVSPDGIIGHSTGEMGCGYADGGITREQTMRLAYHRGTTIMKHTEIKGAMAAVGLTWEEVKAQAPPGVVAACHNGADSVTISGDAEGVASFCAQLKEKEIFAKVVDTSGIPFHSPAMLAVKDEMIESMRTAVPEPKPRSSKWISTSIPEEDWESDLAATCSAEYHVHNACSPVLFYEALQKIPANAVTIEMAPHSLMQAILRRSLQKTVTNVGLMNKPKSENDNELEGFLGSLGKIYQAGVNIQISELYPGGQYKGVVPKGTPMIGPMWQWDHSQDWQTIDGRHMVAGGGGSIPASATYNIDPFAADSKEAYLLDHVIDGRVLYPFTGHMVLAWKTLCKLKSLDFQKTPVVFENINVYSATIVTKPIKLDVVLTPGNGYFEIISDEQVAASGRIYVPEDNQPFYYGKLEDIRTSEIADRIELDTEDAYKEFLLRGYEYGQAFRGIYKTCNSGERGYLYWTGNWVTFLDSLLQTALLAERSDTLRLPTRVRHLRIDPNKHLEHVVERDGIQVVELRNDHSTNGCIAGGVECCDLNAHSVARRIQVSGQLYHEKVFFVPHFDTNCLKQHNETAATLKDYTSVIKQTLFNGFSAWKSAGLLKKLKNGALIEKALPALKSSLSDVVLDDTVTRFMHDGKCTVLPHFTEMFKIQDSEDFEDQVIGKLKSVRGIFELDRLWAGAVLNDRIVKSLQDICIENSAGHHAKMAAVDLVSTDQIRHCIEANSSHPLLEVDYTCVGATVDHLDESTLEIVGAKKQKIVLDEAFTGHGELKNMDYVLLDKVLNKKADPIAFIEACKNLIRDDGFLLVVEVTAQYEIALAIEALLGTDLAGDANRQYGQFFTHEQLLKMFETTGFKICNYQSDPALMTTTYAVRRIASVPRDPAFIDVDDIKEFSWIEPLQKTVEERLNEPDAKTIWLVSNKCRNNGIVGLALCFVEENLKSNRFRSAFDMSATKAIRDGPPVWNIGDEETKKIIDLDMHANDYMDGQWGSMRHIVVKDEDAHVYKDCEHAFINTLTRGDVSSLTWFESPNQYFDSMAKTKSSQELCSVYYAPINFRDIMLAYGRLPPDAIPGNFADRECLLGMEFSGRLKDGTRLMGILPAQALATTVMVDRDYAWEVPSDWTLAEASTVPVVYTTAYYALVRRGMMKKGDKVLIHGGAGGVGQAAIAIALAAGCEVFTTVGSTEKREFLKKLFPQLKEHHFANSRSADFELHIRQHTKGRGVNIVLNSLANEMLQASLRCLARHGRFLEIGKVDLSQNSSLGMAKLLDNVSVHGILLDSIMDPTVGDLEEWKEIARLLEQGIKSGVVKPLHSHSFPADKAEEAFRFMSAGKHIGKVIMEIRQEETTKVCPPSRISVRAICRTLCHPQHTYLITGGLGGFGLELAQWLINRGARKLVLTSRTGIRTGYQARCVHFWRRTGVSILVSTLNIAKKSDAVELVKQCTAMGPVGGVFHLAMVLRDCLFENQNIQNFKDAAEAKYYGTINLDYATREDTVKEHLKWFVVFSSITSGRGNAGQTNYGWSNSCMERMIDQRRADGFPGIAIQWGAIGDVGVILENMGDNNTVVGGTLPQRMPSCLSSLDNFLSWNHPIVSSYIKADMGAKKSAGGGNLMQTIAHILGVNDISQLNPDANLGDLGLDSLMGVEIKQALERDYDIVLSMKDIRTLTLNKLQQLAEQGGTGGTALQVNELEMKKDGERDAELNTVEMLEKQMNQLFKMRVDVNDLDPQDVIVKANKAEEGPITFFVHSIEGIATPLKKVMSKCEFPVYCFQATKNVPQTSIEDVAKYYIREMKKVQPTGPYRLVGYSYGACIGFEMACMLQQSDGQDAVEKLVLLDGSHLYMQTYRNVYRMAFGVTGDTLVNNPLFESEIMCAMTLRFANVDYKKFRVELLQQPGFKARVQKVVDTVMTTGLFKSPETVAFACMAMHSKFLMADKYKPERKFRGHITLIRAEQGAAREEDVGRDYGISQVSDDSDVHVVEGDHDTFVQGKSSAKTVSIINSLILNN

SEQ ID NO: 28 - protein, 2587 aa, Caenorhabditis remanei
MESNSWWQKQDDIVISGVSGRFPRCDNVKEFGDLLLAGEDLVTEDDLRWPPGFYDLPKRHGKLKELKKFDAGFFQVTPKQANFMDPQVRILLEASWEAMVDAGINPIDLRGSKTGVFVGCSASETSGALTQDPESVTGYTLTGCVRSMFSNRISYTFDLQGPSFSVDTACSSSLLALQLAIDSIRQGQCDAALVAGAHLTLTPTAALQFLRLGMLTDKGSCRSFDDSGDGYCRTEGVAAIFIQRKKKAQRIYATVLHAKSNTDGYKEQGITFPSGERQAQLLQEVYSEAGVDPNSVYYVESHGTGTKVGDPQEANAICQVFCSNRSDSLLIGSVKSNMGHAEPASGVCSLTKILLAIERQMIPPNLHYNTPNQYIPGLVDGRLKVVTEPTPLPGGVIGVNSFGFGGSNTHVILRAADHVAPAIHQHPFTKIVTYCGRTQESVEKMFAEIEANKENLYLQALLANQANMPANLLPFRGYMLLDRENAQSPMKNITKVPITEPRPIYFIYSGMGSQWPGMAIKLMKIPMFDESLRASSKTLDEYGLDVYGMLCNPDPEQYSNNTMNCMLAITAIQIALTDTLTALGVSPDGIIGHSTGEMGCGYADGGITREQTMRLAYYRGTTIMNHTEIKGAMAAVGLTWEEVKAQAPKGVVAACHNGADSVTISGDAEGVATFCAQLKEKDIFAKVVDTSGIPFHSPAMLAVKDEMIESMRTAVPEPKPRSSKWISTSIPEEDWESDLAATCSAEYHVHNACSPVLFYEALQKIPANAVTIEMAPHSLMQAILRRSLMKTVTNVGLMNKPKSENDDELESFLGSLGKIYQAGVNIQISELYPGGQYKGVVPKGTPMIGPMWQWDHSQDWQTIDGRHMVAGGGGSIPASATYNIDPFAADSKEAYLLDHCIDGRVLYPFTGHMVLAWKTLCKLKSLDFQKTPVVFENINVYSATIVTKPIKLDVVLSPGNGYFEIISDEQVAASGRIYVPEDNQPFYYGKLEDIRTSEIADRIELDTEDAYKEFLLRGYEYGQAFRGIYKTCNSGERGYLYWTGNWVTFLDSLLQTALLAERSDTLRLPTRVRHLRIDPNKHLEHVVERDGIQVVELRNDHSTNGCIAGGVECCDLNAHSVARRIQVSGQLYHEKVYFVPHFDQNCLKEQPETSTILKDYAAVIKQTLYNGFSKWQKAGLLKKLTNGPLIEKALPVLKSSLNEVVLDDTVTRFMHDGKCTILPHFTELFKIPDSDDFAEMVIGKLKSVRGIFELDRLWAGAVINDRIVKSLQDICIENSAGHHAKMAAVDLVSTDQIRHCIEANSSHPLLEVDYMCVGANVDHLDESTLEIVGGKKQKIVLDENFTGHGEVKNMDYVLLDKVLNKKANPIAFIEACKHLIRDDGFLLVVEVTSQFEIALALESLLGNEMAGDADRKYTQFFTHEQLLEIFKATGFKICNFQSDPALMTTTYAVRRVAPVPRDPAFIDVDDIKEFSWIEPLQKTVEERLNEPDSKTIWLVSNKCRNNGIVGLALCFVEENLKSNRFRSAFDMSATKAIRDGPPVWTIDDEETKKIIDLDLHANDYMDGQWGSMRHIVVKDEDAHVYKDCEHAFINTLTRGDVSSLTWFESPNQYFDSMAKTKSSQELCSVYYAPINFRDIMLAYGRLPPDAIPGNFADRECLLGMEFSGRLKDGTRLMGILPAQALATTVMVDRDYAWEVPSDWTLAEASTVPVVYTTAYYALVRRGMMKKGDKVLIHGGAGGVGQAAIAIALAAGCEVFTTVGSAEKRQFLKNLFPQLQEHHFANSRSADFELHIRQHTKGRGVNIVLNSLANEMLQASLRCLARHGRFLEIGKVDLSQNSSLGMAKLLDNVSIHGILLDSIMDPTVGDLEEWKEIARLLEQGIKSGVVKPLHSHSFPANKAEEAFRFMSAGKHIGKVIMEIRTEESTKICPPSRISVRAICRTLCHPQHVYLITGGLGGFGLELAQWLINRGARKLVLTSRTGIRTGYQARCVHFWRRTGVSVLVSTLNISKKSDAIELVKQCTQMGPLGGVFHLAMVLRDCLFENQNIQNFKDAAEAKYYGTINLDYATRDHSDKETLKWFVVFSSITSGRGNAGQTNYGWSNSTMERMIDQRRADGFPGIAIQWGAIGDVGVILENMGDNNTVVGGTLPQRMPSCLSSLDNFLSWNHPIVSSFIKADMGAKKSAGGGNLMQTIAHILGVNDISQLNPDANLGDLGLDSLMGVEIKQALERDYDIVLSMKDIRTLTLNKLQQLAEQGGTGGTALQVNELEMKKDGERDAELNTVEMLEKQMNQLFKMRVDVNDLDPQDIIVKANKVEEGPITFFVHSIEGIATPLKKVMSKCEFPVYCFQSTKNVPQTSIEDVAKCYIREMKKIQPTGPYRLIGYSYGACIGFEMACMLQQSDGQDAVEKLVLLDGSHLYMQTYRNVYRMAFGVTGDTLVNNPLFESEIMCAMTLRFANVDYKKFRVELLQQSGFKARVQKVVDTVMTTGLFKSDETVAFACMAMHSKFLMADKYKPERKFRGHITLIRAEQGAAREEDVGRDYGISQVSDDSDVHVVEGDHDTFVQGKSSAKTVSIINSIILNK

SEQ ID NO: 29 - protein, 319 aa, Mus musculus
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 30 - protein, 319 aa, Rattus norvegicus
SQRPLWFICSGMGTQWRGMGLSLMRLDSFRESILRSDEALKPLGVKVSDLLLSTDEHTFDDIVHSFVSLTAIQIALIDLLTSMGLKPDGIIGHSLGEVACGYADGCLSQREAVLAAYWRGQCIKDANLPAGSMAAVGLSWEECKQRCPPGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHVPEHAVVLEIAPHALLQAVLKRGVKPSCTIIPLMKRDHKDNLEFFLTNLGKVHLT

SEQ ID NO: 31 - protein, 319 aa, Cricetulus griseus
SKRPLWFICSGMGTQWRGMGLSLMRLDSFRESILRSDEAVKPLGIKVSDLLLSTDESTFDDIVHAFVGLTAIQIALIDLLTSVGLKPDGIIGHSLGEVACGYADGCLSQREAVLAAYWRGQCIKDAHLPPGSMAAVGLSWEECKQRCPSGVVPACHNSEDTVTISGPQAAVSEFVEQLKQEGVFAKEVRTGGLAFHSYFMEGIAPMLLQALKKVIREPRPRSARWLSTSIPEAQWQSSLARTSSAEYNVNNLVSPVLFQEALWHVPEHAVLLEIAPHALLQAVLKRGVKSSCTIIPLMKRDHKDNLEFFLTNLGKVHLT

SEQ ID NO: 32 - protein, 319 aa, Homo sapiens
GERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLS

SEQ ID NO: 33 - protein, 319 aa, Macaca mulatta
GERPLWFICSGMGTQWRGMGLSLMRLDRFRDSILRSDEAVKPFGLKVSQLLLSMDESTFDDIVHAFVSLTAIQIGLIDLLSCMGLRPDGIIGHSLGEVACGYADGCLSQEEAVLAAYWRGQCIKEAHLPPGAMAAVGLSWEECKQRCPPGVVPACHNSKDTVTISGPQASVFEFMEQLRKEGVFAKEVRTGGMAFHSYFMEAIAPPLLQALKKVIREPKPRSARWLSTSIPEAQWHSSLARTSSAEYNVNNLVSPVLFQEALCHVPEHAVVLEVAPHALLQAVLKRGLKPGCTIIPLMKKDHRDNLEFFLTGIGRLHLS

SEQ ID NO: 34 - protein, 319 aa, Bos taurus
GKRPLWFICSGMGTQWRGMGLSLMRLSRFRDSILRSDEAVKPLGLQVSQLLLSTDEAIFDDMVISFVSLTAIQIALIDLLTSMGLQPDGIIGHSLGEVACGYADGCISQEEAILSAYWRGQCIKEANIPPGAMAAVGLTWEECKQRCPPGIVPACHNCIDTVTISGPQASMLEFVQQLKQEGVFAKEVRTGGMAFHSYFMDAIAPMLLQQLKKVIREPQPRSPRWLSTSIPETQWQESLARTFSAEYNVNNLVSPVLFQEALWRVPEDAVVLEIAPHALLQAVLKRGLKSSCTIIPLMKKDHRDNLEFFLSNVGQLYLT

SEQ ID NO: 35 - protein, 318 aa, Capra hircus
SKRPLWFICSGMGTQWRGMGLSLMRLSSFRDSILRSDEAVKPLGLRVSQLLLSADDIFDDIIVSFVSLTAFQIALIDLLTSMGLRPDGIIGHSLGEVACAYADGCISQEEAILSAYWRGQCIKEANIPPGAMAAVGLTWEECKQRCPPGIVPACHNSIDTVTISGPQASMLEFVKQLKQEGVFAKEVQTGGMAFHSYFMDAIAPTLLQQLKKVIREPQLRSPRWLSTSIPESQWHESLARTFSAEYNVNNLVSPVLFQEALWHVPENAVVLEIAPHALLQAILKRGLQPSCTIIPLMKKDHRDNLEFFLSNVGQLYLT

SEQ ID NO: 36 - protein, 319 aa, Equus caballus
GQRPLWFICSGMGAQWRGMGLSLMRLGTFRDSILRSDKAVKPLGLQVSDLLLSADEATFDDTVPGFVSLTAIQIALIDLLTSMGLRPDGIIGHSLGEVACGYADGCVSQEEAILAAYWRGQCIKEANIPPGAMAAVGLSWEECKQRCPPGVVPACHNSKDTVTISGPQAAVSEFVEQLKQEGVFAKEVRTGGLAFHSYFMDSISHTLLQALKKVIREPRPRSVRWLSTSIPEAQWQSSLARTFSAEYNVNNLVSPVLFQEALCHVPEHAVVLEIAPHALLQAVLKRGLKPSCTIVPLMKKDHRDNLEFFLSNVGRLHLM

SEQ ID NO: 37 - protein, 319 aa, Felis catus
GKRPLWFICSGMGAQWCGMGLSLMRLGSFRDSILRSDEAVKPLGLQVSELLLSTDEATFDDIIHAFVSLTAIQIALIDLLNSMGLRPDGIIGHSLGEVACGYADGCLSQEEAILAAYWRGQCIKETNIPPGTMAAVGLSWEECKQRCPPGVVPACHNSEDTVTISGPQAEVAAFVAELKREGVFAKEVRTGGMAFHSYFMDSIAPTLLQALKKVIREPRPRSARWLSTSIPEAQWQGSLARTFSAEYNVNNLVSPVLFQEALWHVPGDAVVLEIAPHALLQAVLKRGLKSSCTIVPLMKKDQRDNLEFFLSNVGKLHLL

SEQ ID NO: 38 - protein, 319 aa, Sus scrofa
SKRPVWFICSGMGAQWQGMGLSLMRLDRFRDSILRSDQALKPLGLRVSDLLLSTDEAVLDDIVSSFVSLTSIQIALIDLLTSLGLQPDGIIGHSLGEVACGYADGCLTQEEAVLSSYWRGYCIKEANVLPGAMAAVGLSWEECKQRCPPGIVPACHNSKDTVTISGPQAAMSEFLQQLKREDVFVKEVRTGGIAFHSYFMESIAPTLLRQLRKVILDPKPRSKRWLSTSIPEAQWQGSLARTFSAEYSVNNLVSPVLFQEALQHVPAHAVVVEIAPHALLQAVLKRSLESSCTIIPLMKKDHRDNLEFFLSNVGRLHLA

SEQ ID NO: 39 - protein, 319 aa, Cuculus canorus
TGRPLWYICSGMGTQWKGMGLSLMKLDLFRQSVLRSDEALKHTGLKVSELLLNEDENTFDDTVHAFVGLAAIQIAQIDILKAAGLEPDGIVGHSVGELACGYADNSLSHEEAILAAYWRGRCVKDAKLPPGGMAAVGLSWEECKQICPPNVVPACHNSEDTVTVSGPLGAVSEFVAKLKKDGVFAKEVRTSGVAFHSHYMASIAPVLLSALKKVIPHPKPRSPRWISTSIPESQWQSDLARYSSAEYHVNNLVNPVLFHEGLSHVPGNALVVEIAPHALLQAILKRSLKPTCIILPLMKKEHKNNLEFFLTQLGKFHMS

SEQ ID NO: 40 - protein, 319 aa, Calypte anna
SGRPLWYICSGMGTQWKGMGLSLMKLDLFRQSILRSDQALKNTGLKVSDLLLQADDNTFDETVHAFVGLAAIQIAQIDMLKAAGLQPDGILGHSVGELACGYADNCLSHEEAILAAYWRGRCVKEAKLPPGGMAAVGLTWEECKQQCPPNVVPACHNSEDTVTISGPLATVSEFVAKLKKAGVFAKEVRSAGVAFHSHYMASIAPVLLSALKKVIPHPKPRSARWISTSIPESQWQSDLAKYSSAEYHVNNLVSPVLFHEGLKHIPENAVVVEIAPHALLQAILKRTLKPTCTILPLMKKDHKNNLEFFLTQAGKIHLT

SEQ ID NO: 41 - protein, 319 aa, Anas platyrhynchos
SGRPLWYICSGMGTQWKGMGLSLMKLDVFRQSIMRSDEALKNTGLKVSDLLLQADDNTFDETVNAFVGVAAVQIAQIDMLKAAGLQPDGILGHSVGEVACGYADNSLSHEEAILAAYWRGRCVKEAKLPPGGMAAVGLTWEECKQRCPPNVVPACHNSEDTVTISGPLDSVNEFVAKLKKDGVFAKEVRSAGVAFHSYYMASIAPALLSALRKVIPHPKPRSARWISTSIPESQWQSDLARNSSAEYYVNNLVSPVLFHEGLKHIPENAVVVEIAPHALLQAILRRSLKPSCTILPLMKKDQKNNLEFFLTQAGKIHLT

SEQ ID NO: 42 - protein, 319 aa, Gallus gallus
SGRPLWYICSGMGTQWKGMGLSLMKLDLFRQSILRSDEALKSTGLKVSDLLLNADENTFDDTVHAFVGLAAIQIAQIDVLKAAGLQPDGILGHSVGELACGYADNSLSHEEAVLAAYWRGRCVKEAKLPPGGMAAVGLTWEECKQRCPPNVVPACHNSEDTVTVSGPLDSVSEFVTKLKKDGVFAKEVRRAGVAFHSYYMASIAPALLSALKKVIPHPKPRSARWISTSIPESQWQSDLARNSSAEYHVNNLVNPVLFHEGLKHIPENAVVVEIAPHALLQAILRRTLKPTCTILPLMKKDHKNNLEFFLTQTGKIHLT

SEQ ID NO: 43 - protein, 319 aa, Nestor notabilis
SGRPLWYICSGMGSQWKGMGLSLMKLDMFRQSILRSDEALKNTGLKVSDLLLRADENTFDETINAFVGLAAIQIAQIDLLKAAGLQPDGILGHSVGELACGYADNCLSQEEAILGAYWRGRCVKEAKLPPGAMAAVGLTWEECKQRCPPNVVPACHNSEDTVTVSGPLESVTEFVAKLKKDGVFAKEVRSAGVAYHSHYMASIAPVLLNALKKVIPHPKPRSARWISTSIPESQWQSDLARSCSAEYLVNNLVNPVLFHDGLKHVPENAVVVEIAPHALLQAILRRTLKPTCTILPLMKKDQKNNLEFFLTQTGKIHLT

SEQ ID NO: 44 - protein, 319 aa, Podiceps cristatus
SGRPLWYICSGMGTQWKGMGVDLMKLDLFRQSILRSDEALKSTGLKVSDLLLQADENTFDDTVHAFVGLAAIQIAQIDMLKAAGLQPDGILGHSVGELACGYADNSLSREEAILAAYWRGRCVKEAKLPPGGMAAVGLTWEECKQCCPPNVVPACHNSEDTVTISGPLDSVHEFVAKLKKDGVFAKEVRSAGVAFHSYYMASIAPVLLSALKKVIPHPKPRSARWISTSIPESQWQSDLARNSSAEYHVNNLVNPVLFHEGLKHVPENAVVVEIAPHALLQAILRRTLKPTCTILPLMKKDHKNNLEFFLTQTGKIHLT

SEQ ID NO: 45 - protein, 319 aa, Balearica regulorum
  source 1..319: note = Balearica regulorum gibbericeps
SGRPLWYICSGMGTQWKGMGLSLMKLDLFRQSILRSDEALKNTGLKVSDLLLQADENTFDDTVHAFVGLAAIQIAQIDMLKAAGLQPDGILGHSVGELACGYADNSLSHEEAILAAYWRGRCVKEAKLPPGGMAAVGLTWEECKQCCPPNVVPACHNSEDTVTVSGPLDSVNEFVAKLKKDGVFAKEVRSAGVAFHSYYMASIAPVLLSALKKVIPHPKPRSARWISTSIPESQWQSDLARNSSAEYHVNNLVNPVLFHEGLKHVPENAVVVEIAPHALLQAILRRTLKPTCTILPLMKKEHKNNLEFFLTQTGKIHLT

SEQ ID NO: 46 - protein, 319 aa, Phaethon lepturus
SDRPLWYICSGMGTQWKGMGLSLMKLDLFRQSILRSDEALKNTGLKVSDLLLQADENTFDDTVHAFVGLAAIQIAQIDMLKAAGLQPDGILGHSVGELACGYADNSLSHEEAILAAYWRGRCVKEAKLPPGGMAAVGLTWEECKQCCPPNVVPACHNSEDTVTVSGPLDSVNEFVAKLKKDGVFAKEVRSAGVAFHSYYMASIAPVLLSALKKVIPHPKPRSARWISTSIPESQWQSDLARNSSAEYHVNNLVNPVLFHEGLKHIPENAVVVEIAPHALLQAILRRTLKPTCTILPLMKKEHKNNLEFFLTQTGKIHLT

SEQ ID NO: 47 - protein, 319 aa, Phoenicopterus ruber
SGRPLWYICSGMGTQWKGMGLSLMKLDLFRQSILRSDEALKNTGLKVSDLLLQADENTFDDTVHAFVGLAAIQIAQIDLLKAAGLQPDGILGHSVGELACGYADNSLSHEEAILAAYWRGRCVKEAKLPPGGMAAVGLTWEECKQCCPPNVVPACHNSEDTVTVSGPLDSVREFVAKLKKDGVFAKEVRSAGVAFHSYYMASIAPVLLSALKKVIPHPKPRSARWISTSIPESQWQSDLARNSSAEYHVNNLVNPVLFQEGLKHIPENAVVVEIAPHALLQAILRRALKPTCTILPLMKKEHKNNLEFFLTQTGKIHLT

SEQ ID NO: 48 - protein, 319 aa, Ooceraea biroi
EKRPIWFVFSGMGTQWSGMGRELLGIEVFRRTLQRCADTLLPHGVDLMNIILNNTNETLENVIDTFVAIAAMQVALIDVLAAIDIYPDGIAGHSVGELGCAYADGAFTAEQTVLAAYWRGKSIVDSTLEPGAMAAVGLSWEDAQKMCPPNVSPACHNSMDSVTISGPTKDVLKFVEELKSKNIFAKLVRSSGIAFHSKYIASAGPKLRASLDKIIPNPKQRSAKWISSSIPEAAWGSPLAQVSSSAYHVNNLLSPVLFQEAIAHIPDNAITIEIAPHCLLQAILRRSLPSTVTNVSLHMRDHTNNLAFLLSNIGKLYMA

SEQ ID NO: 49 - protein, 319 aa, Anopheles funestus
DKRPVWFIYSGMGSQWASMAKDLMQLEVFHNSIYRCAEALRPEGVDLIDVLTKSDDTKFDNILNSFISIAAVQVALTDVLTHVGITPDGMVGHSVGELGCAYADGCFTPEQTVLAAYWRGRSILDTDLIAGQMAAVGLSWEECKQKLPKDVIPACHNSADSVTISGPVNSVGKVIADLNAQGIFAKGVKSSGIAFHSRYIADAAPKLRKSLDKIIPNPKNRTPRWISTSIPEESWPTPLAQQSSSAYHVNNLLSPVLFSEGLKHVPANAICIEIAPHGLLQAILKRALGKDATNLSLMKRDHANNMIFLLSNLGKLYAA

SEQ ID NO: 50 - protein, 319 aa, Anopheles darlingi
EKRPLWFIYSGMGSQWASMARDLMQLEVFHSTIARCAEALRPEGIDLIDVLTKSDESRFDNILNSFISIAAVQVALTDVLTHVGIVPEGMVGHSVGELGCAYADGCFTPEQTVLAAYWRGRSILDTDLIAGQMAAVGLSWEDCKQKLPKDVIPACHNSSDSVTISGPVASVGKVIADLNAQGIFAKGVKSSGIAFHSRYIADAAPKLRKSLDKIIPNPKPRTQRWISTSIPEESWGTALAQQSSSAYHVNNLLSPVLFAEGLKHVPANAICIEIAPHGLLQAILKRALGKEATNLSLMKRDHDNNLIFLLSNLGKLYAA

SEQ ID NO: 51 - protein, 319 aa, Glossina austeni
ESRPIWFVYSGMGSQWASMAKDLMVFDTFKKTIHRCCEILRPEGLDLMEVLTRSTEKTFENILNSFVGIAAMQVALTDLLTSLNIVPDGIVGHSVGEVACAYADGCLSMEQTILAAYWRGKSILDTKLTKGKMAAVGLSWEEAHKRLPADCFPACHNSAENCTISGPEESIDAVCQKLTAEGVFARAVKSSGYAFHSKYIADAGPKLRKSLEKVIPNAKNRSPRWISSSIPEVAWNTAIAQQASAAYHVNNLLSPVLFHQALQHVPKNAICIEVAPTGLLQAILKRSLGNETTNLSLIKRDYENNPEFFLASIGKLYAA

SEQ ID NO: 52 - protein, 319 aa, Drosophila melanogaster
DKRPIWYIYSGMGSQWASMAKDLMKIEAFAKTIQRCADVLKPEGVDLIDVLTRSTDKSFENILNSFISIAAMQVALTDLLSSLGIHPDGIVGHSVGELGCAYADGCFTPEQTVLAAYWRGKSILDTQLAKGKMAAVGLSWEDAHSRVPSDCFPVCHNSEDNCTISGPEASIEALVAKLNAEGVFAKAVNSSGYAFHSKYIAEAGPKLRKSLEKIIPNAKNRTARWISTSIPESAWNTPVAKQSSAAYHVNNLLSPVLFHEALQHVPKNAISVEIAPHGLLQAILKRALGPDATNLSLVKRGHENNVEFFLTNVGKLFAA

SEQ ID NO: 53 - protein, 320 aa, Brugia malayi
EPKQIYFIYSGMGSQWAGMVRQLMSIPAFDESLRSSSDAIVDFGVNVYEMLQSDDPSFYKNNTLNCMLAITAIQIALTDVLFLLGVMPDGIIGHSTGEMCCGYADGGLTREQTMQLAYHRGHTIMNANIKGGMAAVGLSWEEAGKRCPEGVIPACHNAADSVTISGDAEKIKEFVEELKKEDIFGKLVDSSGIPFHSPAMLKVKDKMLKAMRTSVPNPKPRSSRWISTSIPESNWENELAQMCSADYHTNNAVSPVLFYEALQKIPANAVTIEIAPHCLMHSILRRSLQKTCTNVGLINMKEKDRELESFLQALGKIYQT

SEQ ID NO: 54 - protein, 323 aa, Caenorhabditis elegans
EARPIYFIYSGMGSQWPGMAIKLMKIPMFDDSLRASSKTLEEFGLDVYGMLCNPDPEQYSNNTMNCMLAITAIQIALTDVLTALGVSPDGIIGHSTGEMGCGYADGGITREQTMRLAYHRGTTIMKHTEIKGAMAAVGLTWEQVKEQAPPGVVAACHNGADSVTISGDAEGVATFCAQLKEKDIFAKVVDTSGIPFHSPAMLAVQDEMIECMRTAVPEPKPRSSKWISTSIPEDDWESDLAATCSAEYHVHNACSPVLFYEAIQKIPANAVTIEMAPHSLMQAILRRSLQKTVTNVGLMNRPKSENDDELESFLGSLGKIYQA

SEQ ID NO: 55 - protein, 323 aa, Caenorhabditis briggsae
EPRPIYFIYSGMGSQWPGMAIELMKIPMYDESLRASSKTLEEFGLDVYGMLCNPDPEQYSNNTMNCMLAITAIQIALTDTLTALGVSPDGIIGHSTGEMGCGYADGGITREQTMRLAYHRGTTIMKHTEIKGAMAAVGLTWEEVKAQAPPGVVAACHNGADSVTISGDAEGVASFCAQLKEKEIFAKVVDTSGIPFHSPAMLAVKDEMIESMRTAVPEPKPRSSKWISTSIPEEDWESDLAATCSAEYHVHNACSPVLFYEALQKIPANAVTIEMAPHSLMQAILRRSLQKTVTNVGLMNKPKSENDNELEGFLGSLGKIYQA

SEQ ID NO: 56 - protein, 323 aa, Caenorhabditis remanei
EPRPIYFIYSGMGSQWPGMAIKLMKIPMFDESLRASSKTLDEYGLDVYGMLCNPDPEQYSNNTMNCMLAITAIQIALTDTLTALGVSPDGIIGHSTGEMGCGYADGGITREQTMRLAYYRGTTIMNHTEIKGAMAAVGLTWEEVKAQAPKGVVAACHNGADSVTISGDAEGVATFCAQLKEKDIFAKVVDTSGIPFHSPAMLAVKDEMIESMRTAVPEPKPRSSKWISTSIPEEDWESDLAATCSAEYHVHNACSPVLFYEALQKIPANAVTIEMAPHSLMQAILRRSLMKTVTNVGLMNKPKSENDDELESFLGSLGKIYQA

SEQ ID NO: 57 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 58 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDYVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 59 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 60 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAAAEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 61 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 62 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 63 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLDRGVKSSCTIIPLMKRDHKDNLEFFLTNLGKVHLT

SEQ ID NO: 64 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLERGVKSSCTIIPLMKRDHKDNLEFFLTNLGKVHLT

SEQ ID NO: 65 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKDDHKDNLEFFLTNLGKVHLT

SEQ ID NO: 66 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKEDHKDNLEFFLTNLGKVHLT

SEQ ID NO: 67 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKDDHKDNLEFFLTNLGKVHLT

SEQ ID NO: 68 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKEDHKDNLEFFLTNLGKVHLT

SEQ ID NO: 69 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKDDHKDNLEFFLTNLGKVHLT

SEQ ID NO: 70 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKEDHKDNLEFFLTNLGKVHLT

SEQ ID NO: 71 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQVVLKRGVKSSCTIIPLMKRDHKDNLEFFLTNLGKVHLT

SEQ ID NO: 72 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQLVLKRGVKSSCTIIPLMKRDHKDNLEFFLTNLGKVHLT

SEQ ID NO: 73 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVVLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 74 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVLLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 75 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDVVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 76 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLT

SEQ ID NO: 77 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVLAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKLGVKSSCTIIPLMKRDHKDNLEFFLTNLGKVHLT

SEQ ID NO: 78 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVLAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKLGVKSSCTIIPLMKRDHKDNLEFFLTNLGKVHLT

SEQ ID NO: 79 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKDDHKDNLEFFLTNLGKVHLT

SEQ ID NO: 80 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKEDHKDNLEFFLTNLGKVHLT

SEQ ID NO: 81 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKDDHKDNLEFFLTNLGKVHLT

SEQ ID NO: 82 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKEDHKDNLEFFLTNLGKVHLT

SEQ ID NO: 83 - protein, 214 aa, Mus musculus
  source 1..214: note = Sequence of SEQ 1 in Table 1
AAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKRGVKSSCTIIPLMKRDHKDNLEFFLTNLGKVHLTGINVNPNALFPPVEFPAPRGTPLISPHI

SEQ ID NO: 84 - protein, 214 aa, Rattus norvegicus
  source 1..214: note = Sequence of SEQ 2 in Table 1
AAVGLSWEECKQRCPPGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHVPEHAVVLEIAPHALLQAVLKRGVKPSCTIIPLMKRDHKDNLEFFLTNLGKVHLTGIDINPNALFPPVEFPVPRGTPLISPHI

SEQ ID NO: 85 - protein, 214 aa, Cricetulus griseus
  source 1..214: note = Sequence of SEQ 3 in Table 1
AAVGLSWEECKQRCPSGVVPACHNSEDTVTISGPQAAVSEFVEQLKQEGVFAKEVRTGGLAFHSYFMEGIAPMLLQALKKVIREPRPRSARWLSTSIPEAQWQSSLARTSSAEYNVNNLVSPVLFQEALWHVPEHAVLLEIAPHALLQAVLKRGVKSSCTIIPLMKRDHKDNLEFFLTNLGKVHLTGIDVNPNALFPPVDFPAPRGTPLISPHI

SEQ ID NO: 86 - protein, 214 aa, Homo sapiens
  source 1..214: note = Sequence of SEQ 4 in Table 1
AAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLI

SEQ ID NO: 87 - protein, 214 aa, Macaca mulatta
  source 1..214: note = Sequence of SEQ 5 in Table 1
AAVGLSWEECKQRCPPGVVPACHNSKDTVTISGPQASVFEFMEQLRKEGVFAKEVRTGGMAFHSYFMEAIAPPLLQALKKVIREPKPRSARWLSTSIPEAQWHSSLARTSSAEYNVNNLVSPVLFQEALCHVPEHAVVLEVAPHALLQAVLKRGLKPGCTIIPLMKKDHRDNLEFFLTGIGRLHLSGIDANPNALFPPVEFPAPRGTPLISPLI

SEQ ID NO: 88 - protein, 214 aa, Bos taurus
  source 1..214: note = Sequence of SEQ 6 in Table 1
AAVGLTWEECKQRCPPGIVPACHNCIDTVTISGPQASMLEFVQQLKQEGVFAKEVRTGGMAFHSYFMDAIAPMLLQQLKKVIREPQPRSPRWLSTSIPETQWQESLARTFSAEYNVNNLVSPVLFQEALWRVPEDAVVLEIAPHALLQAVLKRGLKSSCTIIPLMKKDHRDNLEFFLSNVGQLYLTGIDVNPNGLFPPVEFPAPRGTPLISPHI

SEQ ID NO: 89 - protein, 214 aa, Capra hircus
  source 1..214: note = Sequence of SEQ 7 in Table 1
AAVGLTWEECKQRCPPGIVPACHNSIDTVTISGPQASMLEFVKQLKQEGVFAKEVQTGGMAFHSYFMDAIAPTLLQQLKKVIREPQLRSPRWLSTSIPESQWHESLARTFSAEYNVNNLVSPVLFQEALWHVPENAVVLEIAPHALLQAILKRGLQPSCTIIPLMKKDHRDNLEFFLSNVGQLYLTGIDVNPNGLFPPVEFPAPRGTPLISPHI

SEQ ID NO: 90 - protein, 214 aa, Equus caballus
  source 1..214: note = Sequence of SEQ 8 in Table 1
AAVGLSWEECKQRCPPGVVPACHNSKDTVTISGPQAAVSEFVEQLKQEGVFAKEVRTGGLAFHSYFMDSISHTLLQALKKVIREPRPRSVRWLSTSIPEAQWQSSLARTFSAEYNVNNLVSPVLFQEALCHVPEHAVVLEIAPHALLQAVLKRGLKPSCTIVPLMKKDHRDNLEFFLSNVGRLHLMGIDVNPNGLFPPVEFPAPRGTPLISPHI

SEQ ID NO: 91 - protein, 214 aa, Felis catus
  source 1..214: note = Sequence of SEQ 9 in Table 1
AAVGLSWEECKQRCPPGVVPACHNSEDTVTISGPQAEVAAFVAELKREGVFAKEVRTGGMAFHSYFMDSIAPTLLQALKKVIREPRPRSARWLSTSIPEAQWQGSLARTFSAEYNVNNLVSPVLFQEALWHVPGDAVVLEIAPHALLQAVLKRGLKSSCTIVPLMKKDQRDNLEFFLSNVGKLHLLGFDVNPNGLLPPVEFPVPRGTPLISPHI

SEQ ID NO: 92 - protein, 214 aa, Sus scrofa
  source 1..214: note = Sequence of SEQ 10 in Table 1
AAVGLSWEECKQRCPPGIVPACHNSKDTVTISGPQAAMSEFLQQLKREDVFVKEVRTGGIAFHSYFMESIAPTLLRQLRKVILDPKPRSKRWLSTSIPEAQWQGSLARTFSAEYSVNNLVSPVLFQEALQHVPAHAVVVEIAPHALLQAVLKRSLESSCTIIPLMKKDHRDNLEFFLSNVGRLHLAGVSVNPNGLFPPVEFPAPRGTPLISPHX

SEQ ID NO: 93 - protein, 214 aa, Cuculus canorus
  source 1..214: note = Sequence of SEQ 11 in Table 1
AAVGLSWEECKQICPPNVVPACHNSEDTVTVSGPLGAVSEFVAKLKKDGVFAKEVRTSGVAFHSHYMASIAPVLLSALKKVIPHPKPRSPRWISTSIPESQWQSDLARYSSAEYHVNNLVNPVLFHEGLSHVPGNALVVEIAPHALLQAILKRSLKPTCIILPLMKKEHKNNLEFFLTQLGKFHMSGVNFNGNNLFPRVEYPVPVETPLISPHI

SEQ ID NO: 94 - protein, 214 aa, Calypte anna
  source 1..214: note = Sequence of SEQ 12 in Table 1
AAVGLTWEECKQQCPPNVVPACHNSEDTVTISGPLATVSEFVAKLKKAGVFAKEVRSAGVAFHSHYMASIAPVLLSALKKVIPHPKPRSARWISTSIPESQWQSDLAKYSSAEYHVNNLVSPVLFHEGLKHIPENAVVVEIAPHALLQAILKRTLKPTCTILPLMKKDHKNNLEFFLTQAGKIHLTGINVLGNNLFPPVEYPVPVGTPLISPYI

SEQ ID NO: 95 - protein, 214 aa, Anas platyrhynchos
  source 1..214: note = Sequence of SEQ 13 in Table 1
AAVGLTWEECKQRCPPNVVPACHNSEDTVTISGPLDSVNEFVAKLKKDGVFAKEVRSAGVAFHSYYMASIAPALLSALRKVIPHPKPRSARWISTSIPESQWQSDLARNSSAEYYVNNLVSPVLFHEGLKHIPENAVVVEIAPHALLQAILRRSLKPSCTILPLMKKDQKNNLEFFLTQAGKIHLTGINVLGNNMFPSVEYPVPVGTPLISPYI

SEQ ID NO: 96 - protein, 214 aa, Gallus gallus
  source 1..214: note = Sequence of SEQ 14 in Table 1
AAVGLTWEECKQRCPPNVVPACHNSEDTVTVSGPLDSVSEFVTKLKKDGVFAKEVRRAGVAFHSYYMASIAPALLSALKKVIPHPKPRSARWISTSIPESQWQSDLARNSSAEYHVNNLVNPVLFHEGLKHIPENAVVVEIAPHALLQAILRRTLKPTCTILPLMKKDHKNNLEFFLTQTGKIHLTGINVLGNNLFPPVEYPVPVGTPLISPYI

SEQ ID NO: 97 - protein, 214 aa, Nestor notabilis
  source 1..214: note = Sequence of SEQ 15 in Table 1
AAVGLTWEECKQRCPPNVVPACHNSEDTVTVSGPLESVTEFVAKLKKDGVFAKEVRSAGVAYHSHYMASIAPVLLNALKKVIPHPKPRSARWISTSIPESQWQSDLARSCSAEYLVNNLVNPVLFHDGLKHVPENAVVVEIAPHALLQAILRRTLKPTCTILPLMKKDQKNNLEFFLTQTGKIHLTGINVLGNNLFPLVEYPVPVGTPLISPYI

SEQ ID NO: 98 - protein, 214 aa, Podiceps cristatus
  source 1..214: note = Sequence of SEQ 16 in Table 1
AAVGLTWEECKQCCPPNVVPACHNSEDTVTISGPLDSVHEFVAKLKKDGVFAKEVRSAGVAFHSYYMASIAPVLLSALKKVIPHPKPRSARWISTSIPESQWQSDLARNSSAEYHVNNLVNPVLFHEGLKHVPENAVVVEIAPHALLQAILRRTLKPTCTILPLMKKDHKNNLEFFLTQTGKIHLTGINVLGNNLLPLVEYPVPVGTPLISPYI

SEQ ID NO: 99 - protein, 214 aa, Balearica regulorum
  source 1..214: note = Sequence of SEQ 17 in Table 1
AAVGLTWEECKQCCPPNVVPACHNSEDTVTVSGPLDSVNEFVAKLKKDGVFAKEVRSAGVAFHSYYMASIAPVLLSALKKVIPHPKPRSARWISTSIPESQWQSDLARNSSAEYHVNNLVNPVLFHEGLKHVPENAVVVEIAPHALLQAILRRTLKPTCTILPLMKKEHKNNLEFFLTQTGKIHLTGINVLGNNLFPLVEYPVPVGTPLISPYI

SEQ ID NO: 100 - protein, 214 aa, Phaethon lepturus
  source 1..214: note = Sequence of SEQ 18 in Table 1
AAVGLTWEECKQCCPPNVVPACHNSEDTVTVSGPLDSVNEFVAKLKKDGVFAKEVRSAGVAFHSYYMASIAPVLLSALKKVIPHPKPRSARWISTSIPESQWQSDLARNSSAEYHVNNLVNPVLFHEGLKHIPENAVVVEIAPHALLQAILRRTLKPTCTILPLMKKEHKNNLEFFLTQTGKIHLTGINVLGNNLFPLVEYPVPVGTPLISPYI

SEQ ID NO: 101 - protein, 214 aa, Phoenicopterus ruber
  source 1..214: note = Sequence of SEQ 19 in Table 1
AAVGLTWEECKQCCPPNVVPACHNSEDTVTVSGPLDSVREFVAKLKKDGVFAKEVRSAGVAFHSYYMASIAPVLLSALKKVIPHPKPRSARWISTSIPESQWQSDLARNSSAEYHVNNLVNPVLFQEGLKHIPENAVVVEIAPHALLQAILRRALKPTCTILPLMKKEHKNNLEFFLTQTGKIHLTGINVLGNNLFPLVEYPVPVGTPLISPYI

SEQ ID NO: 102 - protein, 214 aa, Ooceraea biroi
  source 1..214: note = Sequence of SEQ 20 in Table 1
AAVGLSWEDAQKMCPPNVSPACHNSMDSVTISGPTKDVLKFVEELKSKNIFAKLVRSSGIAFHSKYIASAGPKLRASLDKIIPNPKQRSAKWISSSIPEAAWGSPLAQVSSSAYHVNNLLSPVLFQEAIAHIPDNAITIEIAPHCLLQAILRRSLPSTVTNVSLHMRDHTNNLAFLLSNIGKLYMAGAQPNISKLYPPVSFPVGRGTPMIGPLV

SEQ ID NO: 103 - protein, 214 aa, Anopheles funestus
  source 1..214: note = Sequence of SEQ 21 in Table 1
AAVGLSWEECKQKLPKDVIPACHNSADSVTISGPVNSVGKVIADLNAQGIFAKGVKSSGIAFHSRYIADAAPKLRKSLDKIIPNPKNRTPRWISTSIPEESWPTPLAQQSSSAYHVNNLLSPVLFSEGLKHVPANAICIEIAPHGLLQAILKRALGKDATNLSLMKRDHANNMIFLLSNLGKLYAAGVQPQVQKLYRPITYPVGRGTPMLNSLV

SEQ ID NO: 104 - protein, 214 aa, Anopheles darlingi
  source 1..214: note = Sequence of SEQ 22 in Table 1
AAVGLSWEDCKQKLPKDVIPACHNSSDSVTISGPVASVGKVIADLNAQGIFAKGVKSSGIAFHSRYIADAAPKLRKSLDKIIPNPKPRTQRWISTSIPEESWGTALAQQSSSAYHVNNLLSPVLFAEGLKHVPANAICIEIAPHGLLQAILKRALGKEATNLSLMKRDHDNNLIFLLSNLGKLYAAGAQPQVQKLYPPITYPVGRGTPMLNSLV

SEQ ID NO: 105 - protein, 214 aa, Glossina austeni
  source 1..214: note = Sequence of SEQ 23 in Table 1
AAVGLSWEEAHKRLPADCFPACHNSAENCTISGPEESIDAVCQKLTAEGVFARAVKSSGYAFHSKYIADAGPKLRKSLEKVIPNAKNRSPRWISSSIPEVAWNTAIAQQASAAYHVNNLLSPVLFHQALQHVPKNAICIEVAPTGLLQAILKRSLGNETTNLSLIKRDYENNPEFFLASIGKLYAAGAQPQIMTLSKPISYPVGRGTPMLGCKV

SEQ ID NO: 106 - protein, 214 aa, Drosophila melanogaster
  source 1..214: note = Sequence of SEQ 24 in Table 1
AAVGLSWEDAHSRVPSDCFPVCHNSEDNCTISGPEASIEALVAKLNAEGVFAKAVNSSGYAFHSKYIAEAGPKLRKSLEKIIPNAKNRTARWISTSIPESAWNTPVAKQSSAAYHVNNLLSPVLFHEALQHVPKNAISVEIAPHGLLQAILKRALGPDATNLSLVKRGHENNVEFFLTNVGKLFAAGAQPQVLTLVRPISYPVGRGTPMLNSKV

SEQ ID NO: 107 - protein, 215 aa, Brugia malayi
  source 1..215: note = Sequence of SEQ 25 in Table 1
AAVGLSWEEAGKRCPEGVIPACHNAADSVTISGDAEKIKEFVEELKKEDIFGKLVDSSGIPFHSPAMLKVKDKMLKAMRTSVPNPKPRSSRWISTSIPESNWENELAQMCSADYHTNNAVSPVLFYEALQKIPANAVTIEIAPHCLMHSILRRSLQKTCTNVGLINMKEKDRELESFLQALGKIYQTGITIHIEALYPAIQYPVPIGTPMISPMW

SEQ ID NO: 108 - protein, 219 aa, Caenorhabditis elegans
  source 1..219: note = Sequence of SEQ 26 in Table 1
AAVGLTWEQVKEQAPPGVVAACHNGADSVTISGDAEGVATFCAQLKEKDIFAKVVDTSGIPFHSPAMLAVQDEMIECMRTAVPEPKPRSSKWISTSIPEDDWESDLAATCSAEYHVHNACSPVLFYEAIQKIPANAVTIEMAPHSLMQAILRRSLQKTVTNVGLMNRPKSENDDELESFLGSLGKIYQAGVNIQITELYPGGQYKGVVPKGTPMIGPMW

SEQ ID NO: 109 - protein, 219 aa, Caenorhabditis briggsae
  source 1..219: note = Sequence of SEQ 27 in Table 1
AAVGLTWEEVKAQAPPGVVAACHNGADSVTISGDAEGVASFCAQLKEKEIFAKVVDTSGIPFHSPAMLAVKDEMIESMRTAVPEPKPRSSKWISTSIPEEDWESDLAATCSAEYHVHNACSPVLFYEALQKIPANAVTIEMAPHSLMQAILRRSLQKTVTNVGLMNKPKSENDNELEGFLGSLGKIYQAGVNIQISELYPGGQYKGVVPKGTPMIGPMW

SEQ ID NO: 110 - protein, 219 aa, Caenorhabditis remanei
  source 1..219: note = Sequence of SEQ 28 in Table 1
AAVGLTWEEVKAQAPKGVVAACHNGADSVTISGDAEGVATFCAQLKEKDIFAKVVDTSGIPFHSPAMLAVKDEMIESMRTAVPEPKPRSSKWISTSIPEEDWESDLAATCSAEYHVHNACSPVLFYEALQKIPANAVTIEMAPHSLMQAILRRSLMKTVTNVGLMNKPKSENDDELESFLGSLGKIYQAGVNIQISELYPGGQYKGVVPKGTPMIGPMW

SEQ ID NO: 111 - protein, 319 aa, synthetic construct; identical to SEQ ID NO: 78

SEQ ID NO: 112 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 113 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVAAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 114 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVAADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 115 - protein, 319 aa, synthetic construct; identical to SEQ ID NO: 79

SEQ ID NO: 116 - protein, 319 aa, synthetic construct; identical to SEQ ID NO: 81

SEQ ID NO: 117 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVLADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKLGVKSSCTIIPLMKRDHKDNLEFFLTNLGKVHLT

SEQ ID NO: 118 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVLADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKLGVKSSCTIIPLMKDDHKDNLEFFLTNLGKVHLT

SEQ ID NO: 119 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDDVTISGPQAAVNEFVEQLKQEGVAADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 120 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDDVTISGPQAAVNEFVEQLKQEGVAAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 121 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVEADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 122 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDAVTISGPQAAVNEFVEQLKQEGVEAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 123 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVEADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 124 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVEAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 125 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVEADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKDDHKDNLEFFLTNLGKVHLT

SEQ ID NO: 126 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDLVTISGPQAAVNEFVEQLKQEGVEAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKDDHKDNLEFFLTNLGKVHLT

SEQ ID NO: 127 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDDVTISGPQAAVNEFVEQLKQEGVEADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 128 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDDVTISGPQAAVNEFVEQLKQEGVEAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 129 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDDVTISGPQAAVNEFVEQLKQEGVEADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKDDHKDNLEFFLTNLGKVHLT

SEQ ID NO: 130 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDDVTISGPQAAVNEFVEQLKQEGVEAEEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKDDHKDNLEFFLTNLGKVHLT

SEQ ID NO: 131 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSERAVTISGPQAAVNEFVEQLKQEGVEADEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKDGVKSSCTIIPLMKRDHKDNLEFFLTNLGKVHLT

SEQ ID NO: 132 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKAGVKSSCTIIPLMKRDHKDNLEFFLTNLGKVHLT

SEQ ID NO: 133 - protein, 319 aa, synthetic construct
NKRPLWFICSGMGTQWRGMGLSLMRLDSFRESILRSDEAVKPLGVKVSDLLLSTDERTFDDIVHAFVSLTAIQIALIDLLTSVGLKPDGIIGHSLGEVACGYADGCLSQREAVLAAYWRGQCIKDAHLPPGSMAAVGLSWEECKQRCPAGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHIPEHAVVLEIAPHALLQAVLKLGVKSSCTIIPLMKRDHKDNLEFFLTNLGKVHLT